(19) United States
(12) Patent Application Publication
(10) Pub. No.: US 2010/0304357 A1
(43) Pub. Date: Dec. 2, 2010

(54) UNIVERSAL DRINKING ADAPTER FOR BEVERAGE BOTTLES, AND DEVICES AND KITS FOR DETERMINING SMALL MOLECULES, METAL IONS, ENDOTOXINS, AND BACTERIA, AND METHODS OF USE THEREOF

(76) Inventors: Steven Meyers, Raleigh, NC (US); Michel Wathier, Brighton, MA (US); Mark Grinstaff, Brookline, MA (US)

Correspondence Address: LANDO & ANASTASI, LLP, ONE MAIN STREET, SUITE 1100, CAMBRIDGE, MA, 02142, US

(21) Appl. No.: 12/718,604

(22) Filed: Mar. 5, 2010

Related U.S. Application Data
(63) Continuation-in-part of application No. PCT/US08/75552, filed on Sep. 8, 2008.
(60) Provisional application No. 60/970,306, filed on Sep. 6, 2007.

(51) Int. Cl. G01N 33/04; A61J 9/04; A47G 19/22; B65D 69/00; G01N 33/06; C12M 1/00; G01N 21/00; C12Q 1/00

(52) U.S. Cl. 435/4; 215/11.5; 220/711; 436/22; 206/569; 436/23; 422/68.1; 435/287.1; 422/82.05

(57) ABSTRACT

Certain features, aspects, examples and embodiments described herein relate to adapters for securing drinking apparatuses for individuals of all ages (infants, children, adults, and seniors) such as nipples, sippers, and straws, to commercially available beverage containers to aid in the consumption of the contained liquid. Other features, aspects, examples and embodiments relate to devices and kits useful for the detection of analytes in milk samples such as small molecules, metal ions, endotoxins, and bacteria.

### PRIORITY APPLICATION

This application is a continuation-in-part of International PCT Application No. PCT/US08/075552 filed Sep. 8, 2008, which claims priority to U.S. Provisional Application No. 60/970,306 filed on Sep. 6, 2007, the entire disclosure of which is hereby incorporated herein by reference for all purposes.

### TECHNOLOGICAL FIELD

Certain features, aspects, examples and embodiments described herein relate to adapters for securing drinking apparatuses for individuals of all ages (infants, children, adults, and seniors) such as nipples, sippers, and straws, to commercially available beverage containers to aid in the consumption of the contained liquid. More specifically, certain embodiments relate to a bottle adapter that does not engage the external threading on the neck of the bottle by providing complementary threading but contains a mechanism for combined internal and external fixation. Other features, aspects, examples and embodiments relate to devices and kits useful for the detection of analytes in milk samples such as small molecules, metal ions, endotoxins, and bacteria. More specifically, certain other features, aspect, examples and embodiments relate to the detection of fatty acids, mercury, endotoxins, and bacterial acidity in samples of human milk

### BACKGROUND

The feeding of an infant using a standard wide-mouth baby bottle is a method of hydration. These bottles provide a well-defined environment in which the liquid is held and allow for reliable dispensing of the liquid through an attached adapter, generally a rubber nipple. These bottles are generally adapted and used by tightening a rubber nipple with a flange between a threaded annular ring and corresponding threading on the neck of the bottle. However, such bottles may prove to be inconvenient for out-of-home use where a parent or guardian would be required to transport appropriate amounts of the prepared liquid in addition to the typical infant accessories. These liquid filled bottles can be bulky and cumbersome, may leak, and, depending on the liquid used, may spoil or obtain an unacceptable temperature putting the child at risk due to the lack of appropriate thermal regulation.

Commercial beverage bottles are widely available and so would prove to be a convenient alternative to liquid carried along from home. These bottles usually constructed from plastic or glass to contain such beverages as water, juice, milk, or soda are usually sold sterilized and could readily be used to hydrate infants or any individual when away from home. Unfortunately, these bottles utilize a threaded cap over a circular opening and so do not present an obvious mechanism whereby an infant can take a drink, and for older children, these bottles are readily spillable. Additionally, unlike with aluminum beverage cans, there is not a consistent mouth size present among products and so methods of adapting an infant's drinking nipple to fit the opening can prove to be difficult as each manufacturer will generally use a different sized mouth and threading pattern.

With respect to related prior art, previous inventions in the adaption of bottles for infant use can be divided into two broad classifications: those that engage the outer portion of the bottle neck, generally by interacting with the threading, and those that secure themselves with friction by snugly fitting into the mouth of the bottle. Herein we describe and demonstrate a new design that is accomplished by engaging both the interior and exterior of the bottle neck to maximize the type of bottles that can be adapted. Thus we describe the use of a unitary adapter and the mechanism by which it functions. This dual fixation mechanism may lead to higher required pull-off forces, which would provide increased safety for the child. In some embodiments of this invention the unitary construction of the adapter, with the drinking apparatus intrinsically adhered to the bottle adapter, will greatly simplify the adaption by not requiring a plurality of pieces.

U.S. Pat. No. 6,851,565 of Stephan describes an annular adapter that contains internal threading to engage the external threads of a commercially available beverage bottle and accommodates the addition of a standard baby bottle annular tightening ring and nipple inserted therein. U.S. Pat. No. 6,354,449 of Smith utilizes a similar design where again, an annular hard plastic ring containing internal threads engages the threading on the neck of a bottle and provides external threading on the same piece in which to engage a standard baby bottle annular clamp ring and rubber nipple inserted therein. U.S. Pat. No. D414,873 of Kwiecinski uses a similar design with an annular ornamental ring containing internal threads that engage the external treads on the bottle neck and provides a second set of external threads on a separate part to engage the standard baby bottle annular clamp ring and rubber nipple inserted therein. U.S. Pat. No. 5,024,341 again utilizes a similar design where an annular plastic ring contains internal threading to engage the external threading on a bottle neck in addition to containing a separate external set of threads that engage a clamp ring to secure a rubber nipple to the invention. U.S. Pat. No. 6,666,345 of Blanding describes a combination baby bottle cap that can also engage the exterior threading of commercially available beverage bottles. The five inventions listed above all engage the commercial water bottles by a mechanism that utilizes internal threading to engage the external male threads on the bottle neck. To strongly engage the threading of these bottles a rigid plastic/polymer is necessitated, but commercially available beverage bottles come in a wide variety of neck sizes and threading structures, with some having no threads, limiting the amount of bottles that each adapter would properly interact with. In view of the above mentioned limitations, an improvement was recognized by the present inventors that a flexible adapter that did not engage the threading but relied on a mechanism such as frictional force to maintain a secure closure over the opening would be beneficial. Such a design would fit a variety of bottle sizes and so the user would not need to find a specific adapter fit but instead could rely on a single product to adapt most available bottles. As outlined below, this adaption will be accomplished using a large flexible flange that can be extended over the exterior of the bottle neck to prevent liquid leakage and maintain a frictional grip on the bottle.

A different system to engage the interior of the bottle neck has also been reported. Prior art of this type is known, specifically in regards to liquor dispensation (U.S. Pat. Nos. 2,800,241, 3,422,998, 3,434,636, 3,595,421). In the field of adapting baby bottles for infant consumption U.S. Pat No. 7,185,775 of Decal involves an axial passage that is inserted into the neck of a bottle, with the axial passage containing resilient annular rings that surround the passage. The portion of the invention that remains outside of the neck contains external threading to allow for the use of a conventional bottle nipple and clamp ring. U.S. Pat. No. 2,771,073 of Mills describes the use of a solid plug containing a rubber or rubber-like sealing mechanism that is inserted into the neck of a vacuum or “thermos” bottle that accepts a nursing nipple. U.S. Pat. No. 1,623,544 of Kushner describes a similar invention where a plug containing a cork sheath is friction fit into the neck of a vacuum bottle which also contains a nursing nipple to allow for liquid consumption. U.S. Pat. No. 177,185 of Whitney describes a similar idea where a glass stopper containing male threads is screwed into the neck of a glass bottle containing internal female threading within the neck. The stopper contains an appropriate apparatus to allow for the withdrawal of the fluid. The present inventors realized the above named inventions that engage the interior of a bottle neck all suffer from the drawback of solely relying on internal friction fitting to supply the necessary interaction strength. Because this interaction needs to be strong, the designs rely on fitting the stopper into bottles with a narrow range of adaptable neck diameters, thereby limiting the potential use with commercially available beverage bottles which employ a myriad of neck designs. An improved and novel method to accomplish fitting of a wide variety of bottle types realized by the present inventors is to use a tapered stopper containing resilient annular rings in addition to the external flange outlined above. This allows a snug fit to a wide variety of bottle designs, with the adapter gaining increased pull-off resistance from the secondary external adapter described in the previous paragraph.

Milk is produced by the mammary glands of female mammals and is the primary source of nutrition for newborns and infants. Milk consists of a micro emulsion of fat suspended in a solution of casein, albumin, milk sugar, and inorganic salts. A typical sample of human mother's milk can contain anywhere between 1 to about 18% fat. A fat content of 5 wt % is considered normal or ideal and, in fact, this is the concentration of fat in milk supplements. The fat constituent of breast milk is the glycerol based lipids which are composed of many types of fatty acids. Breast milk may also contain a variety of nonmetals and metals including antimony (Sb), arsenic (As), cadmium (Cd), calcium (Ca), chlorine (Cl), chromium (Cr), cobalt (Co), copper (Cu), fluorine (F), iodine (I), iron (Fe), lead (Pb), magnesium (Mg), manganese (Mn), mercury (Hg), molybdenum (Mo), nickel (Ni), phosphorus (P), potassium (K), selenium (Se), sodium (Na), tin (Sn), vanadium (V), and zinc (Zn) and also contains other biologic contaminants such as bacteria, endotoxins, and viruses.

Breast milk provides optimal nutrition for the young infant and supports general health, growth and development, while reducing the risk and/or severity of diseases including: diarrhea,¹⁻³ respiratory tract infection,^{4,5} urinary tract infection,⁶ otitis media,^{7,8} and necrotising enterocolitis.⁹ Unfortunately, the same characteristics that make it ideal for proper infant development also make breast milk an excellent food stock for bacterial growth. Excessive amounts of bacteria and their endotoxins in breast milk can be deleterious for infant health. To deal with this reality, new mothers are generally given a rule of thumb regarding pumped milk storage where beyond three days in the refrigerator or one month in the freezer milk is generally regarded as unsafe for consumption.¹⁰ Milk banks in the United States have no such rule that they can abide by regarding the safety of their donated milk samples. Oftentimes they cannot be absolutely sure of the thermal history of the donated milk and so milk samples are screened for bacteria before and after pasteurization, followed by long-term freezing. The bacterial screening involves taking the milk and either sending a sample off to a contract laboratory for further evaluation or in-house culturing on agar plates to determine the bacterial colony count after 48 hours of incubation. Neither of these testing methods has an inherently quick turn-around time and are both labor and cost intensive. The reason for a dual screening is to ensure that firstly there is not an undue bacterial load before pasteurization and secondly that no bacteria survived the pasteurization process.

Endotoxins in milks and formulas fed to infants have been shown to increase the permeability of the gut to bacterium.¹¹⁻¹³ The increased intestinal permeability is associated with a range of symptoms including fever, low blood pressure, inflammation, sepsis and has been suggested as a possible explanation of sudden infant death syndrome (SIDS).¹⁴⁻¹⁸ The best-practice screening methods currently in use at milk banks and hospitals can determine the amount of live bacteria, but not the amount of endotoxins in the milk sample. This runs contrary to the FDA requirements that all medical devices and injectables receive endotoxin testing to ensure that a minimum of disease causing pathogens are present.¹⁹ Banked milk is most often given to hospital intensive care units (ICU) and though it does not specifically fall into either of the above FDA categories, as a prescribed medical supplement it would benefit from a more rigorous testing procedure to ensure the absence of endotoxins and pyrogens. A more stringent and accurate testing methodology for the pre-pasteurization screening would be to count the amount of endotoxins in the sample as endotoxins are not destroyed by the pasteurization processes.²⁰⁻²²

Bacterium themselves are not inherently pathogenic, but the toxins that they secrete (exotoxins) and the toxins present on their cell walls (endotoxins) are responsible for the illnesses. The classic endotoxins, lipopolysaccharides (LPS), with the terms being used interchangeably, are found on the exterior membrane of gram-negative bacterium and are comprised of a sugar chain and lipid moiety. When present on live bacteria, LPS results in the clinical manifestation of disease commonly associated with bacterial infections. Because the molecular weight of these molecules can vary over 2 orders of magnitude, endotoxin concentrations are measured in endotoxin units (EU). An EU correlates to approximately 100-10,000 bacteria depending on the molecular weight of the LPS involved and the specifics of the particular bacterial species.^{23,24} As would be expected, increases in the number of bacteria result in a corresponding increase in the amount of toxin LPS present in the host organism. Because these toxins are notoriously difficult to destroy, LPS can still promote illness even after the bacterium has been inactivated.²⁰⁻²² It has been reported that even small amounts of endotoxins present on drugs and medical devices can cause fevers, lowering of blood pressure, inflammation, and sepsis upon deployment into an animal.¹⁴⁻¹⁶ Some research has even suggested that endotoxin contaminated milk could play a role in sudden infant death syndrome (SIDS) and endotoxin loads in milk and formula is responsible for increased gut permeability to pathogenic bacteria.^{11-13,17,18}

As mentioned earlier, endotoxins are notoriously difficult to destroy.²⁰⁻²² This fact is recognized by current milk bank testing procedures which seek to indirectly measure the endotoxin concentration before pasteurization by measuring the count of colony forming units.²⁵ Though pasteurization can reliably remove the bacteria themselves, harsh conditions are required to inactivate their endotoxins. Some common methods involve using sodium hydroxide, heating to 250° C. for 30 minutes, or ultra filtration of everything in a sample above 10,000 g/mol. Unfortunately, none of these methodologies are amenable to being used with breast milk as they will remove all of the benefits to using human milk.

There are only two FDA accepted methodologies for the detection of endotoxins in a sample. First, a sample of the unknown substance could be injected into a rabbit to determine if the rabbit develops a fever. However, such a test involves ethical concerns as well as no mechanism to quantify how much endotoxin was present. The second mechanism for endotoxin detection relies on the observation by Bang that horseshoe crabs developed an intravascular coagulation in response to gram negative bacterial infection.²⁶ A protein in the circulating amebocytes of the crabs was determined to be the catalyst of the clotting, and a lysate of these cells, Limulus Amebocyte Lysate (LAL) was found to be an extremely sensitive indicator of endotoxin concentrations.²⁷ The presence of endotoxin either on live bacteria or in solution catalyzes the activation of a proenzyme that is present in the LAL.^{28,29} The rate of activation is dependent on the concentration of the endotoxin with the activated enzyme hydrolyzing bonds within a clotting protein that self-associates forming a network. In 1987 the FDA published guidelines on the usage of LAL as an “End-product Endotoxin Test for Human and Animal Parenteral Drugs, Biological Products, and Medical Devices.”³⁰ The LAL test itself can use 3 different methodologies to determine the outcome. The first two involve an optical aspect such as examining the turbidity or chromogenic change in a sample. The other test uses the presence of absence of a gel-clot to quantify the amount of endotoxin.

Most countries with milk banks have storage and handling guidelines in place to ensure the safety of the supply.³¹⁻³⁴ In general the procedure involves prescreening of the mothers prior to milk donation to guarantee that they are free from potential viral (HIV, CMV, HTLV) and bacterial (TB) diseases. Other stringent requirements are in place regarding diet, smoking, caffeine, and travel restrictions. Mothers that pass these checks are then allowed to donate their frozen milk to the bank. The milk is then thawed, and undergoes a pre-pasteurization bacterial screen to indirectly measure the amount of endotoxin in the sample. After waiting 48 hours for the test results the samples that had below a critical bacterial threshold are mixed and pooled to ensure that the milk from 3-5 mothers is included in the final dispensed batch. This is to ensure consistency in the prescribed milk. The samples undergo Holder Pasteurization where they are heated to 62.5° C. for 30 minutes. As noted earlier, though this pasteurization method virtually eliminates the chances of bacterial and viral infections that could result from the banked milk, it does not destroy the endotoxin load already present in the milk. These pathogenic lipopolysaccharides remain in the milk sample. The pasteurized milk is then sampled by inoculating each bottle on a bacterial growth medium such as agar. These plates are incubated at 37° C. for 48 hours and checked for the presence of bacterial colony formation signifying the presence of active bacteria in the milk sample. If live bacteria are found, the sample from which it came is removed from the supply chain and disposed. The remaining bottles are frozen and stored until they are dispensed to infants. Sometimes an additional culturing check for bacteria is performed before the milk is sent to the end user.

The US milk bank system collects and dispenses a total of 1.75 million ounces of breast milk a year. Each of the dosed milk is stored in a 4 ounce bottle before pasteurization occurs which means that there are potentially over 425,000 samples that need to be screened each year. Using the current plating method of bacterial screening, which takes 2 days to perform, this would require a significant amount of time and is quite labor intensive. Products such as the Petrifilm™ from 3M are available that ensure a consistent testing procedure. In using the product the technician opens the sterilized film, inoculates it with some milk from the sample, and then incubates for 48 hours. After the incubation, the technician removes the plate and examines the film for the development of bacterial colonies. The film contains a red indicator dye that colors these colonies red for easier counting. The two day wait before results are obtained makes this testing technique both time and labor intensive compared to the solution we have devised. Again, this procedure is repeated twice, once before and once after pasteurization. Additionally, because this method only looks for the presence of live bacteria, a significant pyrogenic source in the samples, the endotoxin load, is not directly examined.

Breastfeeding is one of the most important contributors to infant health. It is known that newborns need approximately 500-700 kilocalories per day for normal development; 45-55% of the caloric contribution of milk comes from the fat. The American Academy of Pediatrics (AAP) recommends that an infant be breastfed without supplemental foods or liquids for the first 6 months of life (known as exclusive breastfeeding). Epidemiological research shows that breast feeding provides advantages to infants in terms of general health, growth, and development while reducing the risk and/or severity of diseases, including diarrhea^{1,3,35},¹⁻³ respiratory tract infection^{4,5},⁴ urinary tract infection⁶, otitis media^{7,8} and necrotising enterocolitis⁹. Breast feeding also provides protection against sudden infant death syndrome³⁶, ulcerative colitis, Crohn's disease³⁷ and it may have a significant effect on cognitive development³⁸.

In addition, breastfeeding has shown to help mothers bond with their babies, return to pre-pregnancy body weight, and facilitate the return of the uterus to its normal shape and size. There is also evidence that women who have breastfed their infants have a reduced risk of premenopausal breast cancer³⁹, ovarian cancer⁴⁰, and hip fracture^{41,42} compared with women who did not breastfeed⁴². Research also indicates that breastfeeding may protect against type 1 diabetes.^{43,44} From an economic perspective, a study performed in California in 1996 showed that families could save from $459 to $808 (according to the discount applied) per year, per family if breast milk was used, instead of formula milk.⁴⁵

Despite the importance of breastfeeding,⁴⁶ more than 80% of mothers stop breastfeeding before their babies reach six months of age⁴⁷. In fact only Washington and Alaska had over 20% of infants exclusively breastfed at 6 months with a national average of 11.3% according to a 2007 CDC report. The early interruption of breastfeeding not only affects short- and long-term health outcomes for the mother and child but also exacts a financial toll on the U.S. economy. The costs to our government is estimated to be in excess of $1 billion each year if only diarrhea, respiratory syncytial virus, insulin-dependent diabetes mellitus and otitis media occurring in children who were not breast fed are taken into account.⁴⁸ This toll is potentially much higher on developing economies. It is commonly assumed that the demands on working mothers are the leading cause to early breastfeeding cessation. However, research has shown that the most common reason for mothers to stop breastfeeding is the assumption that their babies are not receiving enough milk. A study conducted by Ahluwalia et al, showed that 28-37% of mothers who stopped breastfeeding believed that they were not producing enough milk, and 10% believed their baby was not gaining enough weight⁴⁹.

The standard procedure to access whether a baby is receiving enough nutrition from breast milk consists of measuring the average volume produced at each feeding session. Modern breast pumps enable mothers to easily and accurately measure the volume of breast milk produced, but there is no easy and affordable test available to measure the fat and/or calorie content of breast milk. Studies have shown that many factors my influence the fat content of breast milk, and that values can vary widely within a feeding session, at different times of the day, and among mothers.

In addition to reassuring mothers, measuring the calorie content of breast milk is crucial in managing low-birth-weight, preterm, and “failure to thrive” infants. In 2004, low-birth weight babies (less than 2,500 grams) represented 8.1 percent of the 4,115,590 US newborns;⁵⁰ preterm babies (less than 37 weeks of gestation) represented 12.5 percent; and about 10% of infants receiving primary care show signs of “failure to thrive” (“height or weight less than the third to fifth percentiles for age on more than one occasion” and/or “fall off' 2 major percentile lines using the standard growth charts of the National Center for Health Statistics”). It is common practice to introduce formula as an easy and accurate way to monitor caloric intake. However, the use of formula deprives babies from the benefits of breast milk described earlier.

An alternative way to assure the adequate calorie intake for underweight, premature, and failure to thrive babies is to separate breast milk into foremilk (expressed early in a breastfeeding session—relatively poor in calorie), and the calorie-rich hind milk (available after 2 or 3 minutes of milk expression). Feeding babies with a higher percentage of hind milk results in higher calorie intake and improves weight gain. Because hind milk fat levels are variable, hospitals currently use technology (creamatocrit) that would be expensive and time-consuming for in-home use to accurately measure its fat and calorie levels.⁵¹ To date there is no home-test available for mothers, leaving the only choice to use formula as a means to accurately measure the calorie intake in an infant.

Lucas et al. first described creamatocrit, a method for determining the percentage of fat and energy content in human milk.⁵² The creamatocrit method consists of measuring the ratio of the lipid layer vs. the milk layer after centrifugation of a milk sample (ratios expressed in units called creamatocrit). Although studies have shown that this technique is suitable for hospital use, the equipment's price, size, and the necessary training create significant barriers to home use.⁵³ Hospital and neonatal intensive care units are using the creamatocrit measurement instead of true lipid measurement to determine the calorie content of breast milk. Among the different creamatocrit devices used, the Creamatocrit plus™ is used by hospitals and neonatal intensive care units. This device is a bench-top centrifuge with a manual caliper requiring three different measurements at the interfaces of sealant/milk, fat/water (“fat” and “cream” are used interchangeably), and fat/air. These measurements are challenging, as the user needs to accurately define the middle of the slanted fat/water and fat/air interfaces. This is particularly difficult at low fat content when a clean border between fat/water and fat/air are not present. Furthermore, this type of device has several disadvantages which reduce its utility as a potential home device: 1) its price: $1700, 2) its accuracy: the company reports a correlation of r=0.95 and r²=0.91 between lipid and creamatocrit, yet the deviation at each individual point can be great as 5 to 8 vol % for a specific lipid concentration, 3) its error associated with the end user (an experienced user is unable to get within 1 creamatocrit % reproducibility between measurements of the same sample), 4) its size and weight which limit user friendliness, and 5) the lower limit/resolution of detection is around 3% or 16.5 g/L.

As mentioned above, breast milk provides optimal nutrition for the young infant and supports general health, growth and development, while reducing the risk and/or severity of diseases including: diarrhea,¹⁻³ respiratory tract infection,^{4,5} urinary tract infection,⁶ otitis media,^{7,8} and necrotising enterocolitis.⁹ Unfortunately, breast milk can also contain trace amounts of heavy metals which if present at higher concentrations can be toxic. Mercury (Hg) is an example of one such toxic metal that can be present in breast milk. Although studies show that prenatal mercury exposure is harmful to the developing brain, causing neuronal atrophy, nursing infants are also at risk due to mercury contained in the breast milk. High mercury consumption in the early years of life can lead to alterations ranging from motor impairment, visual loss, hearing loss, developmental delay, seizures, and severe hypertension.⁵⁴⁻⁵⁶ The U.S. Agency for Toxic Substances and Disease Research (U.S. ATSDR) recommended oral mercury consumption be 2 μg Hg/kg/day for inorganic mercury and 0.3 μg MeHg/kg/day (μg of Hg per kg of body weight) for organic mercury.⁵⁷ The Food and Drug Administration (FDA) has similar recommendations for organic mercury consumption of 0.47 μg MeHg/kg/day.⁵⁷ As will be shown later, the levels of breast milk mercury are highly correlated with the levels in the blood of the mother, so a second and more readily obtainable restriction of 5 μg Hg/kg (μg of Hg per kg of blood) has been set by the EPA for woman aged 18-49.⁵⁸

The discovery of the many health benefits of fish consumption over the last decades has increased the importance of this food source in the diet of U.S. women. There are a number of proven benefits for the mother and the developing baby, such as the intake of iron, vitamin E, selenium, and long-chain n-3 polyunsaturated acids particularly eicosapentaenoic acid and docosahexanoic acid.^{59,60} Indeed, fish ingestion during pregnancy has been show to correlate with better infant cognition.⁵⁹ However, the Hg concentration in some fish species, particularly apex predators with long life spans (e.g., tuna and swordfish) can be quite high due to bioaccumulation, leading to increases in mercury concentration in the mother's blood and subsequently the breast milk after consumption. This mercury is generally in the form of the more toxic methylmercury (MeHg), metabolized from the inorganic form by aquatic bacterial species. In fact, a recent study conducted in California with consumers who reported eating fish on a regular basis showed that 89% of them had blood mercury levels exceeding the U.S. Environmental Protection Agency (U.S. EPA) reference levels (5 parts per billion (ppb; μg of Hg/kg of blood)).⁵⁸

Inorganic mercury can reach the environment and become a pollutant through natural forces (e.g., volcanoes) or through various industrial activities, including: coal-fired power plants, metal smelting and mining, manufacture of electronic devices, incineration of municipal waste streams, and chlorine production. It also can enter the environment through the disposal of products containing mercury, such as batteries, fluorescent bulbs, thermometers and thermostats.^{56,59,61-64} Once this contamination reaches larger bodies of water it is converted by intrinsic bacterial activity into organic mercury, most commonly methylmercury (MeHg). The distinction between these two mercuric species is biologically pronounced with the organic form possessing increased uptake through digestion and increased body residence time.^{65,66} For example, estimates of mercury uptake calculate the digestive absorption of a consumed dose to be only 15% for the inorganic form,⁶⁷ but 95% for methylmercury,^{68,69} which is obviously a vital concern. The organic mercury is readily disseminated through the aquatic food-chain as larger predatory fish consume many smaller and less contaminated food sources. These large predators are the final recipients of this exponential bioaccumulative process and oftentimes have substantial amounts of MeHg (˜1 ppm; 1 mg Hg per kg of fish tissue) (Table 1).

Consumption of mercury has serious hematotoxic, neurotoxic, and nephrotoxic properties. A recent study conducted in California, with consumers who reported eating fish on a regular basis showed that 89% of them had blood mercury levels exceeding the U.S. Environmental Protection Agency (U.S. EPA) reference levels.⁷⁰ A more diverse study of the general US female population between 16 and 49 years of age (n=1709 patients) conducted by the Centers for Disease Control (CDC) showed a more modest 10% of the woman had blood levels over the recommended 5 μg Hg/kg of blood.⁵⁸ However, this study points out that fish consumption is the primary correlative factor determining patient mercury concentrations so regions with high seafood consumption will have a greater need for monitoring.⁷¹⁻⁷³ Mercury ingestion is a growing health concern when balanced against the health benefits of seafood consumption. Consumers in the future will need to carefully consider both the benefits and drawbacks of fish consumption. A recent review of the relevant literature published by the Journal of the American Medical Association recommends that woman of childbearing age limit fish consumption to a modest 1-2 servings/week, a limit at which the cardiovascular benefits still outweigh the risks from mercury.⁷⁴ This serving suggestion is reciprocated by both the EPA and FDA.⁷⁵ Clearly, termination of seafood consumption is not recommended for nursing mothers, but careful, informed consumption is advisable.

Regardless of the source, once organic mercury enters the bloodstream it can remain recirculating for extended periods of time due to a half-life of ˜45 days.⁷⁶⁻⁷⁹ From there the mercury easily enters the breast milk, where total mercury levels are at 30% of those found in maternal blood.^{80,81} As would be expected, a correlation has been observed between total, organic, and inorganic mercury in blood and breast milk which shows a p-value relating these two values of less than one-hundredth of a percent. If maternal blood reaches a high level of mercury, there can be a significant increase of this metal in the breast milk posing risk to the infant.^{62,71} Careful analysis of the mercury composition in both the blood and milk have shown that the percentage of organic mercury tends to be high in both at levels of 74% and 49% respectively, which is of concern for the nursing infant.⁸¹ This number is corroborated by a study done in Iraq evaluating the milk of mothers who were inadvertently exposed to mercury treated wheat. Total mercury in their milk reached concentrations of over 200 μg Hg/kg of milk, with 60% of it comprised of the organic form.⁸² Therefore, approximately half of the mercury load in breast milk is comprised of the more toxic and more readily absorbed organic form, which could have significant consequences for the nursing infant.

Total mercury concentration in milk understandably varies according to amounts of fish consumption. Separate studies in 1976, one in Iowa and one in Alaska, exemplify this dietary correlation. Nursing mothers in Iowa who were tested for milk Hg levels showed an average concentration of 0.9±0.23 μg Hg/kg of milk, which is below the recommendations established by the US ATSDR.⁸³ However, the mothers in Alaska had levels of 3.2±0.8 μg Hg/kg in the interior and 7.6±2.7 μg Hg/kg in costal populations, owing to the larger importance that seafood serves in their diet.⁸⁴ Because half of the mercury present in breast milk is comprised of the more toxic methylmercury these mothers on average had 1.6 and 3.8 μg MeHg/kg of milk, which is an extraordinarily high amount for infant consumption. For example, a 5 kg infant that drinks 1 liter of milk during the feedings over the course of 1 day, has a US ATSDR recommended maximum consumption of 10 μg Hg and 2.35 μg MeHg. A clear majority (64%) of the coastal residents would therefore have an organic mercury load beyond the acceptable limits, with 6% of the inland residents surpassing this requirement as well. The weight of the infant is important in calculating the allowable body burden and we will address this issue with the proposed device. Finally, a more stringent requirement was established by the World Health Organization (WHO) that recommends intake levels for nursing infants should only be ⅓ of the value from the US ATSDR, which would only increase the amount of mothers and infants that are at risk for excess mercury burden and would even switch some of the Iowa mothers into this category as well.⁸⁵

Given the importance of monitoring levels of Hg in high risk populations such as mothers who consume fish on a regular basis or those that live in industrial or coastal areas, we describe the first disposable testing kit for Hg levels in breast milk. This kit will enable earlier detection of breast milk contamination by mercury and could avoid, or at least minimize, intoxication of infants and mothers. In addition to home and clinical use, this kit would have pronounced benefits for the milk received by the nation's 10+ milk banks. This testing kit would provide an efficient and rapid method to screen all incoming milk samples for unacceptable levels of mercury before storing and distributing to at need infants.

A personal test kit for mercury levels does not currently exist on the market. The only similar test available on the market is Boris' Mercury Check™ sold by National Safety Products, Inc. (Finksburg, Md.) for testing mercury concentrations in tap water. The test proceeds by dipping a stick with a color change pad into a large volume of the sample and gently moving it around for 1 minute before a color-change reading is obtained. The user would then compare the color to a gradient printed on the package to determine the amount of mercury in parts-per-billion (ppb). The manufacturer requests a volume of 200 mL or more, which is easy to obtain when testing tap water, but not practical for a nursing mother to provide, especially when the sample has to be wasted after the measurement because the milk has been in contact with chemicals. A conversation with the supplier revealed that smaller volumes do not provide enough mercury to activate the correct color change in the testing pad. Moreover, there is no guarantee that the color change in the milk and water would be proportional. A simple test conducted on mercury-doped milk showed this to be the case confirming that this product is not useful for the determination of mercury in milk.

Testing laboratories for mercury use an atomic absorption method to calculate the amounts of the element in an unknown sample. The sample to be tested is placed into a chamber where it is vaporized into a gaseous phase. A wide-spectrum beam of light is passed through the atomized sample and the amount of absorbance at various spectra is compared against known concentration samples to determine the amounts of specific elements in the sample. This is a simplification of the procedure, but briefly shows how difficult it would be to use to create a home-use, rapid, easy-to-use product based on this technology.

The number of women of child-bearing age (15 through 44 years) in the US is approximately 61,000,000.⁸⁶ Of those, 6%—or 3,746,000—are estimated to have a child in a given year.⁸⁶ Using the study conducted by the CDC which reported 10% of woman between 16 and 49 years of age had blood levels of mercury over the recommended 5 μg Hg/kg of blood, we can estimate that there would be 375,000 women per year whose infants are at risk for increased mercury exposure.⁵⁸ These mothers and infants would benefit from using the kit.

Consequently, there exists a need to detect and monitor the fat and heavy metal content of breast milk as well as to determine if breast milk has spoiled and is no longer ideal for the infant. Embodiments of the present invention describes methods to detect, monitor, and subsequently control the calorie content of breast milk through diet and feeding habits of the mother. Thus, certain embodiments disclosed herein relate to devices and methods for establishing the calorie content of a lactating female's milk as a function of daily food intake, thereby enabling determination of the optimal time for feeding a newborn or infant as well as to ensure normal fat content is being provided to the newborn or infant. Further aspects disclosed herein relate to a monitor or device for measuring the heavy metal content of breast milk. Moreover, a monitor or device for determining if breast milk has spoiled is also described.

### SUMMARY

Certain features, aspects, examples and embodiments described herein provide a bottle adapter constructed and arranged for internal and external fixation to a beverage container. The bottle adapter comprises a plug comprising an internal channel and an external portion comprising at least one annular ring. The bottle adapter can further comprise an external sealing flange connected to the plug and constructed and arranged to be positioned over an exterior portion of the beverage container, and a dispenser in fluid communication with the internal channel of the plug.

In an additional embodiment, the plug is constructed and arranged to insert into an interior portion of a neck of the beverage container, and the at least one annular ring of the external portion of the plug extends axially from the plug and is constructed and arranged to engage an interior portion of the neck of the beverage container. The external sealing flange may have a resting diameter less than a smallest diameter of the neck of the beverage container.

In an additional embodiment, the dispenser comprises a top portion selected from the group consisting of a nipple top, a sipper-type top, a straw top terminated in a nipple top, a straw top terminated in a sipper-type top, a straw top terminated in a tubular straw opening, a secondary internal tube constructed and arranged to allow for liquid withdrawal from the bottom of the beverage container, and combinations thereof.

In an additional embodiment, the adapter may further comprise a base portion, a ring clamp, means for attaching the ring clamp to the base portion, and a top portion secured by the ring clamp consisting of nipple top, a sipper-type top, a straw top terminated in a nipple top, a straw top terminated in a sipper-type top, a straw top terminated in a tubular straw opening, a secondary internal tube constructed and arranged to allow for liquid withdrawal from the bottom of the beverage container, and combinations thereof.

In an additional embodiment, the adapter may be constructed and arranged to interact with a snap-in portion that is constructed and arranged to be inserted into the base portion and secured into place by a closure, wherein the snap-in portion consists of a nipple top, a sipper-type top, a straw top terminated in a nipple top, a straw top terminated in a sipper-type top, a straw top terminated in a tubular straw opening, a secondary internal tube constructed and arranged to allow for liquid withdrawal from the bottom of the beverage container, and combinations thereof.

In an additional embodiment, the adapter further comprises a venting mechanism constructed and arranged to allow for communication between an internal portion of the beverage container and an external environment.

In certain embodiments, a method for determining the fat or caloric content of a breast milk sample by exposing the sample to a surface comprising measuring a property of the breast milk sample and surface interaction is used. In certain examples, the property of the breast milk sample and surface interaction is selected from the group consisting of flowrate, droplet volume, droplet count, droplet timing, droplet contact angle, surface energy relationship between the sample and a surface, and combinations thereof.

In certain other examples, the surface is of a form selected from the group consisting of a channel, groove, tube, and combinations thereof.

In additional embodiments, the method further comprises determining a concentration of the breast milk sample by a technique selected from the group consisting of visual inspection, application of a light source, application of an electrochemical source, application of a sound source, application of a flow counter, application of a speed measurement device, application of a drop counter, application of a drop timer, and combinations thereof.

In additional embodiments, the method further comprises at least one of adding at least one dye to the breast milk sample to aid in visualization, wherein the dye is selected from the group consisting of: litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino) azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R, FD&C Red 3, FD&C Red 40, FD&C Yellow 5, FD&C Yellow 6, FD&C Blue 1, FD&C Blue 2, FD&C Green 3, Caramel Coloring, Annatto, Chlorella, Cochineal, Beet Juice, Saffron, Paprika, Tumeric, Anthrocyanin, Chlorophyll, beta-Carotene, B-Apo-8′-Carotenal, Canthaxanthin, Carrot Oil, Cottonseed Flour, Ferrous Gluconate, Grape Extract, Riboflavin, Carminic Acid, Titanium Dioxide, salts thereof, and combinations thereof; and adding at least one redox active species to increase the conductivity of the milk sample to aid in detection and subsequent determination of the content wherein said species is selected from the group consisting of NaCl, KCl, NaBr, NaI, KBr, KI, ferrocene; tris(2,2′-bipyridine)ruthenium (II); and tris(2,2′-bipyridine)osmium (II), derivatizied ferrocene, methyl violagen, polythiophene, polyanaline, polypyrrole, ruthenium trisbypridine, transitional metal complex, conducting polymer, and combinations thereof.

In certain additional embodiments, the method further comprises adding the sample of breast milk to a vessel constructed and arranged to hold the sample of breast milk to be assayed, and inserting the vessel into a device comprising a detection circuitry.

Other additional embodiments may include a device for testing the caloric or fat content of a breast milk sample comprising a loading reservoir for holding the sample, a detection cell constructed and arranged to allow passage of the sample thereby producing a milk and surface interaction, a catch reservoir for retaining an efflux of the sample, and a detection circuitry constructed and arranged to measure a physical property of the sample and display a response.

Further additional embodiments may include a device for testing a body fluid for analytes comprising a vessel constructed and arranged to hold a sample of the body fluid, a cap for closing the vessel, and at least one material. The material can be selected from the group consisting of a detecting pH sensitive dye, a colorant dye, a base, a solvent to improve solubility, a detecting enzyme, a substrate for an enzyme, and a metabolic activity detecting agent, wherein the at least one material is contained in at least one of the vessel, the cap, a crushable ampoule, and combinations thereof.

An additional embodiment further comprises a component selected from the group consisting of a medicament, colorant, flavoring, scent, fibrous additive, antioxidant, thickener, plasticizer, preservative, stabilizer, and combinations thereof in the at least one of the vessel, the cap, a crushable ampoule, and combinations thereof.

In certain examples, the device is constructed and arranged to analyze a property of the body fluid selected from the group consisting of an acidity of the sample to determine if it is spoiled comprising a pH sensitive detecting agent dye and a base where an incomplete acid-base reaction occurs between the base and the acid in the fluid such that the detecting agent changes, an endotoxin load in the sample to count bacterial levels whereby an enzyme/compound is used to react to the endotoxins in live/dead bacteria, a metabolic activity of the sample to detect spoilage whereby a metabolic detecting agent is used to determine the amount of active bacteria present in the sample, and a concentration of a metal in the sample by using a detecting enzyme/substrate combination that is effected by the presence of a metal wherein said metal is selected from the group consisting of mercury, inorganic mercury, organic mercury, mercury chloride, mercury bromide, mercury acetate, mercury iodide, lead, lead chloride, lead acetate, lead bromide, lead iodide, antimony (Sb), arsenic (As), cadmium (Cd), calcium(Ca), chlorine (Cl), chromium (Cr), cobalt (Co), copper (Cu), fluorine (F), iodine (I), iron (Fe), lead (Pb), magnesium (Mg), manganese (Mn), mercury (Hg), molybdenum (Mo), nickel (Ni), phosphorus (P), potassium (K), selenium (Se), sodium (Na), tin (Sn), vanadium (V), and zinc (Zn).

In certain examples, the detecting agent is selected from the group consisting of a tetrazolium salt, resazurin, methyl blue, dodecylresazurin, RedoxSensor Red, Limulus amoebocyte lysate, litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino) azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R, salts thereof, ferrocene; tris(2,2′-bipyridine)ruthenium (II); and tris(2,2′-bipyridine)osmium (II), derivatizied ferrocene, methyl violagen, polythiophene, polyanaline, polypyrrole, ruthenium trisbypridine, transitional metal complex, conducting polymer, and combinations thereof.

In certain examples, the detecting enzyme selected from the group consisting of mercuric reductase, 1-lactate dehydrogenase, invertase, δ-aminolevulinate dehydrogenase, pyruvate dehydrogenase, alkaline phosphatase, horseradish peroxidase, caspase, and urease, or an oxidoreductase, transferase, hydrolase, lyase, isomerase, ligase, and combinations thereof. In certain other examples, the substrate is selected from the group consisting of urea, NADPH, lactate, pyruvate, sucrose, δ-aminolevulinate acid, para-nitrophenyl phosphate, 2-2′-azino-di-(3-ethylbenz-thiazoline sulfonic acid), o-phenylenediamine, tetramethylbenzidine, a dye bound to the tetrapeptide sequence aspartic acid-glutamic acid-valine-aspartic acid, and combinations thereof.

In other examples, the solvent is selected from the group consisting of pentane, cyclopentane, hexane, cyclohexane, benzene, toluene, 1,4-Dioxane, chloroform, diethyl ether, dichloromethane, tetrahydrofuran, ethyl acetate, dimethylformamide, acetonitrile, dimethyl sulfoxide, formic acid, n-butanol, isopropanol, n-propanole, ethanol, methanol, xylene, ethylene glycol, water, and combinations thereof.

In certain other examples, the base is selected from the group consisting of NaOH, KOH, LiOH, Ca(OH)₂, Ba(OH)₂, Mg(OH)₂, ammonium hydroxide, ammonium citrate, hydroxylamine, pyridine, imidazole, tris amine, triethylamine, NH3, diisopropylethylamine, alanine, dimethylamine, ethylamine, hydrazine, methylethanolamine, methylamine, azetidine, pyrrolidine, piperidine, dimethylethanolamine, diethylamine, aniline, and trimethylamine, and combinations thereof.

In additional embodiments, the device further comprises at least one of a dye added to the milk sample to aid in visualization selected from the group consisting of litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino) azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R, FD&C Red 3, FD&C Red 40, FD&C Yellow 5, FD&C Yellow 6, FD&C Blue 1, FD&C Blue 2, FD&C Green 3, Caramel Coloring, Annatto, Chlorella, Cochineal, Beet Juice, Saffron, Paprika, Tumeric, Anthrocyanin, Chlorophyll, beta-Carotene, B-Apo-8′-Carotenal, Canthaxanthin, Carrot Oil, Cottonseed Flour, Ferrous Gluconate, Grape Extract, Riboflavin, Carminic Acid, Titanium Dioxide, salts thereof, and combinations thereof; and a redox active species to increase the conductivity of the milk sample to aid in detection and subsequent determination of the content selected from the group consisting of NaCl, KCl, NaBr, NaI, KBr, KI, ferrocene; tris(2,2′-bipyridine)ruthenium (II); and tris(2,2′-bipyridine)osmium (II), derivatizied ferrocene, methyl violagen, polythiophene, polyanaline, polypyrrole, ruthenium trisbypridine, transitional metal complex, conducting polymer, and combinations thereof.

In an additional embodiment, a method of testing a body fluid for analytes is used. The method comprises providing a vessel constructed and arranged to hold a sample of the body fluid, providing a cap for closing the vessel, and providing at least one material. The material can be selected from the group consisting of a pH-sensitive dye, a colorant dye, a base, a solvent, an enzyme, a substrate, and a metabolic activity indicator, wherein the at least one material is contained in at least one of the vessel, the cap, a crushable ampoule, and combinations thereof. The method can further comprise adding the body fluid to the vessel, mixing the body fluid and the at least one material to provide a response, and analyzing the response.

In an additional embodiment, the adapter or device further comprises sterilizing the adapter or device using a technique selected from the group consisting of visible light irradiation, ultraviolet light, electron-beam radiation, gamma-radiation, chemical techniques, physical techniques including wet and dry heating, and combinations thereof. In an additional embodiment, a kit is provided, the adapter or device of further comprising at least one of a delivery system selected from to the group consisting of a syringe, spoon, cup, trough, pipette, dropper, and capillary tube, a logbook for recording results, a chart for plotting results, instructions and a URL for a website where results can be interfaced, a desiccant, an antioxidant, means for achieving an inert atmosphere, packaging, means for light blocking, and instructions.

Certain features, aspect, examples and embodiments described herein provide an adapter for individuals of all ages and especially for infants that secures a drinking apparatus to commercially available beverage bottles that does not engage by a corresponding threading mechanism the external threads on the bottle neck but maintains a snug fit using combined external and internal fixation. In certain embodiments, the design fits, or can be adapted to fit, a number of different neck diameters and designs through the use of the external and internal fixation components to provide a generalized adapter for use with a range of commercial beverage containers.

In certain embodiments, the device may include an internal plug, fitting within the mouth of the bottle neck, which engages the smooth walls of the container. Through this plug resides an internal axial passage that allows for the removal of the fluid contained in the bottle. The portion of the adapter residing on the exterior of the bottle neck is comprised of a large flexible flange that can be stretched over the exterior of the bottle neck to provide a secondary means of fixation. The remainder of the design encompasses an attached drinking apparatus that allows for the infant to remove the fluid by means of a nipple, sipper, or straw, thereby comprising unitary construction. Another embodiment of the design involves drinking apparatuses that can be clicked into the external/internal fixation system to provide changeability in the drinking apparatus. In another embodiment of the design a threaded portion on the top of the adapter is provided that allows the device to be used with conventional bottle nipples and annular clamp rings. Another embodiment is the presence of grooves, holes, flaps, flanges, channels, or the use of the tapered plug to allow for the passage of air into the bottle to replace volume of the removed liquid.

Aspects of the present technology relate to devices for measuring the caloric or fat content of milk, for measuring the amount of heavy metals (such as Hg) in breast milk, and for determining if breast milk has spoiled by monitoring the bacteria count, acidity, or endotoxin load. Some embodiments are directed to monitoring the calorie content of breast milk as a function of the mother's food intake in order to know or to optimize the number of calories in her breast milk. In certain embodiments, a process or method of measuring the calorie content in milk either before or after feeding an infant or both, and optionally repeating this procedure such that good nutritional behavior may be adopted is provided. In other embodiments, a closed-looped system that is useful for monitoring and optionally controlling the calorie content of milk, thereby optionally affecting the diet of a newborn or infant is disclosed. Additional embodiments are directed to the detection and measurement of heavy metals like mercury and lead in breast milk. In other embodiments, a method whereby if high concentrations of heavy metals are detected, the mother changes her eating habits to reduce fish consumption or stops breastfeeding and provides formula milk to the infant is provided. Additionally, certain embodiments describe a method to determine if breast milk has spoiled or if the milk contains an undue endotoxin load. Other aspects relate to the provision of kits for conveniently and effectively implementing the methods associated with the devices disclosed herein. These kits can be used in the home, workplace, or on the go.

In one aspect, an adapter that uses internal and external fixation to adapt a beverage container for the intake of liquids by an infant, child, adult, or senior is provided. In some embodiments, the beverage container optionally includes one or more of the following features: (a) the beverage container may be made of plastic, polymer, metal, ceramic, or glass; (b) the beverage container neck may be threaded externally, internally, or neither using a variety of threading patterns or may not possess threading; and/or in which the contained beverage may be water, milk, juice, mineral water, vitamin water, soda, sports drink, breast milk, infant formula added to water, combinations of the above beverages, or another beverage type not explicitly listed here.

In certain embodiments, the adapter comprises one or more of: (a) a solid adapter body comprised of a material such as a rubber, plastic, polymer, ceramic, metal, glass, or natural material such as cork or wax that is inserted into the interior neck of a bottle, wherein the adapter body is shaped with a reducing diameter so that a wide variety of bottle opening styles can be accommodated and wherein non-tapered or slightly tapered embodiments are included; (b) an axial passageway that allows the contained liquid to flow through the adapter; (c) one or more flexible annular rings surrounding the adapter body that engage the sides of the bottle neck by friction and prevent the escape of fluid from the container, wherein the rings are comprised of a flexible rubber, plastic, polymer, wax, or cork material and are constructed with triangular, hemicircular, or rectangular geometries that extend axially from the adapter body; (d) a long flexible flange constructed of rubber, plastic, polymer, cork, or wax that is pulled over the exterior of the bottle neck, further securing the adapter to the bottle and additionally preventing further liquid loss, wherein the resting diameter of the flange is slightly smaller than the smallest diameter bottle opening so that the elastic recoil force tightens around the bottle neck, and wherein the flange is of sufficient length to cover a wide variety of bottle types; and/or (e) zero, one or more reinforcing ribs are manufactured into the flexible flange constructed of the same or different rubber, plastic, or polymer material and will be circumferentially situated around the flange to add tear resistance and elastic strength.

In another aspect, a system that is manufactured unitarily so that a beverage container is secured to an adapter as described is provided. In certain examples, the system may further comprise any one or more of the following: (a) a nipple top; (b) a sipper-type top; (c) a straw top terminated in: (i) a nipple top; (ii) a sipper-type top; and (iii) a tubular straw opening; and/or (d) a secondary internal tube that allows for liquid withdrawal from the bottom of the bottle that can be used in conjunction with any of the previously listed tops.

In other embodiments, the adapter described herein can be manufactured to adapt to a standard baby bottle nipple and ring clamp and can be comprised of one or more of: (a) a solid base portion in addition to the complete adapter described herein that is comprised of a rubber, plastic, polymer, metal, ceramic, wax, or cork; (b) an internal passage that allows the liquid to flow through the portion described in clause (a) in this paragraph; (c) an attachment method that allows a ring clamp to be attached to the base from clause (a) of this paragraph that optionally includes threading, a snap, drawstring, Velcro™ fastener, an adhesive, friction, or a zipper; (d) a standard baby bottle nipple with a base flange to allow the elastomeric nipple to be secured to the bottle adapter; and/or (e) a standard baby bottle ring clamp comprised of a solid material such as a plastic, polymer, rubber, metal, ceramic, or glass that contains an optional internal threaded mechanism or the device of clause (c) of this paragraph, in which the ring clamp may be tightened to the adapter base, securing the nipple to the bottle.

In certain embodiments, the adapter described herein can also be configured to interact with a second snap-in piece comprised of one or more of: (a) the base described in the previous embodiment with an additional solid plastic, rubber, polymer, metal, ceramic, or glass portion that is affixed adjacent to the portion described in clause (a) of the previous embodiment; (b) a second piece that is inserted into the item from clause (b) and secures into place by a snap, tie, knot, Velcro™ fastener, zipper, adhesive, threading, or frictional mechanism, wherein the piece from clause (b) is optionally terminated in a: (i) a nipple top; (ii) a sipper-type top; (iii) a straw top terminated in: (I) a nipple top, (II) a sipper-type top, or (III) a tubular straw opening; (d) an attachment mechanism for a standard baby bottle nipple and clamp ring as described in the previous embodiment; and/or (e) a secondary internal tube that allows for liquid withdrawal from the bottom of the bottle that can be used with any top described above, wherein the secondary pieces may optionally be used interchangeably or may be swapped and secured into the base of the adapter.

In some embodiments, any of the adapters described herein may include a venting mechanism to allow for air intake to relieve pressure developed during the drinking process, in which the venting mechanism optionally comprises any one or more of the following, either alone or in any combination: a hole, a channel, a groove, a flange, and a flap.

In other embodiments, any of the adapters described herein may be manufactured in a variety of sizes to be able to adapt small mouth beverage bottles, large mouth beverage bottles, and infant milk bottles. In additional embodiments, any of the adapters described herein may comprise an included filter to remove a component of the fluid.

In an additional aspect, a kit comprising one or more of the adapters described herein and optionally instructions for use with or without a desiccant or antioxidant is described. In some embodiments, the kit or its components, including the adapter, are disposable, biodegradable, sterilized, reusable with or without sterilization, or recyclable.

In certain embodiments, the kit may be prepared using a sterilization method of the entire kit or components contained therein prior to packaging using one or more of the following methods: (a) visible light irradiation; (b) ultraviolet light irradiation; (c) electron-beam radiation where the amount of radiation is between about 2 and about 40 kGy, about 5 to about 12 kGy, or wherein the radiation is applied more than once; (d) gamma-radiation where the amount of radiation is between about 2 and about 40 kGy, about 3 and about 20 kGy, about 5 and about 12 kGy or wherein the radiation is applied more than once; (e) chemical techniques comprising the use of: (i) ethylene oxide vapors, (ii) hydrogen peroxide vapors; (f) physical techniques including: (i) pressure sterilization, (ii) temperature sterilization with dry heat,(iii) steam sterilization and moist heating or (iv) liquid heating and immersion; and/or (g) any combinations of the techniques listed in sections a-f of this claim, wherein said kit or components contained therein has a sterility assurance level of at least about 10⁻³ or at least about 10⁻⁶.

In another aspect, a concentration-type assay test device for determining if a sample of breast milk has spoiled, comprising a detecting agent is provided. In certain embodiments, the concentration assay for determining if a sample of breast milk has spoiled comprises a detection agent and base. In other embodiments, concentration is determined by visual inspection, application of a light source, or application of an electrochemical source. In some examples, a concentration-type assay test device for determining if a sample of breast milk has spoiled can include a detecting agent wherein said detection agent signals a change in metabolic activity of the sample. In certain examples, the detecting agent is a tetrazolium salt, resazurin, methyl blue, dodecylresazurin, or RedoxSensor Red. In some examples, the detecting agent is in contact with paper, polymer, glass, metal, ceramic, metal oxide, graphite, aqueous solution, alcoholic solution, organic solution, film, porous film, filter, microparticle, nanoparticle, or nanotube. In other examples, the detecting agent and base are in contact with paper, polymer, glass, metal, ceramic, metal oxide, graphite, aqueous solution, alcoholic solution, organic solution, film, porous film, filter, microparticle, nanoparticle, or nanotube. In certain embodiments, contact is achieved through absorption, adsorption, and/or covalent linkage. In some embodiments, the detecting agent is immobilized chemically or by a gel matrix. In additional embodiments, the detecting agent is a solid, dissolved in an aqueous solution, alcoholic, aqueous-alcoholic solution, organic solution, or neat. In other embodiments, the base is a solid, dissolved in an aqueous solution, alcoholic solution, aqueous-alcoholic solution, organic solution, or neat. In certain examples, the aqueous or aqueous-alcoholic solution has an osmotic pressure of about 100 mOs/kg to about 700 mOs/kg. In some examples, the aqueous or aqueous-alcoholic solution has an osmotic pressure of about 200 mOs/kg to about 400 mOs/kg. In certain embodiments, the aqueous or aqueous-alcoholic solution has a pH of about 1 to about 12 or higher, has a pH of about 5 to about 8, or has a pH of about 6 to about 7. In other embodiments, the aqueous or aqueous-alcoholic solution has a pH of about 1 to about 12 following contact with a sample of breast milk, has a pH of about 5 to about 8 following contact with a sample of breast milk, or has a pH of about 6 to about 7 following contact with a sample of breast milk.

In certain embodiments, the detecting agent of any of the devices may be a molecule, macromolecule, or polymer. In some embodiments, the molecule, macromolecule, or polymer is a pH indicator, dye, redox indicator, or metabolic indicator. In certain examples, the detecting agent is selected from the group consisting of: litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino) azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R and salts thereof.

In certain embodiments, more than one detection agent is present.

In other embodiments, a gradient of or two color changes are observed. In some examples, the molecule, macromolecule, or polymer is a redox active species consisting of: a tetrazolium salt, resazurin, methyl blue, dodecylresazurin, or RedoxSensor Red.

In some examples, the detecting agent of any one or more of the devices described herein is selected from the group consisting of ferrocene; tris(2,2′-bipyridine)ruthenium (II); and tris(2,2′ -bipyridine)osmium (II), derivatizied ferrocene, methyl violagen, polythiophene, polyanaline, polypyrrole, ruthenium trisbypridine, transitional metal complex, and conducting polymer.

In other examples, the base of any of the devices described herein is selected from the group consisting of: NaOH, KOH, LiOH, CaOH₂, BaOH₂, MgOH₂, ammonium hydroxide, ammonium citrate, hydroxylamine, pyridine, imidazole, trisamine, triethylamine, NH₃, diisopropylethylamine, alanine, dimethylamine, ethylamine, hydrazine, methylethanolamine, methylamine, azetidine, pyrrolidine, piperidine, dimethylethanolamine, diethylamine, aniline, and trimethylamine.

In one embodiment, the detecting agent is phenolphthalein.

In another embodiment, the solution of detecting agent can be a mixture of both (base and dye) or two different solutions (one base and one dye).

In certain embodiments, the detecting agent is a solid and said base is in solution. In some examples, the base is sodium hydroxide.

In one embodiment, the detecting agent is phenolphthalein and the base is sodium hydroxide.

In another embodiment, the device may include a metabolic detecting agent that is a tetrazolium salt.

In another aspect, a device for testing if breast milk has spoiled comprises a vessel for holding the sample of breast milk which already contains both the detecting agent and base, and a cap for closing the vessel is provided.

In an additional aspect, a device for testing if breast milk has spoiled comprises a vessel for holding the sample of breast milk which already contains the detecting agent and a crushable ampoule containing the base, and a cap for closing the vessel is described.

In another aspect, a device for testing if breast milk has spoiled comprises a vessel for holding the sample of breast milk which already contains the detecting agent, and a cap for closing the vessel which already contains the base, which upon mixing enters the vessel is provided.

In an additional aspect, a device for testing if breast milk has spoiled comprises a vessel for holding the sample of breast milk which already contains the detecting agent, and a cap for closing the vessel which contains one crushable ampoule containing the base, which upon breaking enters the vessel is disclosed.

In another aspect, a device for testing if breast milk has spoiled comprises vessel for holding the sample of breast milk which already contains the base and a crushable ampoule containing the detecting agent, and a cap for closing the vessel is provided.

In an additional aspect, a device for testing if breast milk has spoiled comprises a vessel for holding the sample of breast milk containing a crushable ampoule containing both the detecting agent and the base, and a cap for closing the vessel is described.

In another aspect, a device for testing if breast milk has spoiled comprises a vessel for holding the sample of breast milk containing two crushable ampoules one containing the detecting agent and the other containing the base, and a cap for closing the vessel is provided.

In an additional aspect, a device for testing if breast milk has spoiled comprises a vessel for holding the sample of breast milk containing a crushable ampoule containing the detecting agent, and a cap for closing the vessel which already contains the base, which upon mixing enters the vessel.

In another aspect, a device for testing if breast milk has spoiled comprises a vessel for holding the sample of breast milk containing a crushable ampoule containing the detecting agent, and a cap for closing the vessel which contains one crushable ampoule containing the base, which upon breaking enters the vessel.

In an additional aspect, a device for testing if breast milk has spoiled comprises a vessel for holding the sample of breast milk which already contains the base, and a cap for closing the vessel which already contains the detecting agent, which upon mixing enters the vessel. In another aspect, a device for testing if breast milk has spoiled comprises a vessel for holding the sample of breast milk which already contains the base, and a cap for closing the vessel which contains one crushable ampoule containing the detecting agent, which upon breaking enters the vessel.

In an additional aspect, a device for testing if breast milk has spoiled comprises a vessel for holding the sample of breast milk which contains a crushable ampoule containing the base, and a cap for closing the vessel which already contains the detecting agent, which upon mixing enters the vessel.

In another aspect, a device for testing if breast milk has spoiled comprises a vessel for holding the sample of breast milk which contains a crushable ampoule containing the base, and a cap for closing the vessel which contains one crushable ampoule containing the detecting agent, which upon breaking enters the vessel.

In an additional aspect, a device for testing if breast milk has spoiled comprises a vessel for holding the sample of breast milk, and a cap for closing the vessel which already contains both the detecting agent and base, which upon mixing enter the vessel. In another aspect, a device for testing if breast milk has spoiled comprises a vessel for holding the sample of breast milk, and a cap for closing the vessel which already contains the detecting agent and a crushable ampoule containing the base, which upon breaking and mixing enter the vessel.

In an additional aspect, a device for testing if breast milk has spoiled comprises a vessel for holding the sample of breast milk, and a cap for closing the vessel which already contains the base and a crushable ampoule containing the detecting agent, which upon breaking and mixing enter the vessel.

In another aspect, a device for testing if breast milk has spoiled comprises a vessel for holding the sample of breast milk, and a cap for closing the vessel which contains a crushable ampoule containing both the detecting agent and the base, which upon breaking enter the vessel.

In an additional aspect, a device for testing if breast milk has spoiled comprises a vessel for holding the sample of breast milk, and a cap for closing the vessel which contains two crushable ampoules one containing the detecting agent and the other containing the base, which upon breaking enter the vessel.

In another aspect, a device for testing if breast milk has spoiled comprises a vessel for holding the sample of breast milk which already contains the detecting agent, and a cap for closing the vessel.

In an additional aspect, a device for testing if breast milk has spoiled comprises a vessel for holding the sample of breast milk containing a crushable ampoule containing the detecting agent, and a cap for closing the vessel.

In another aspect, a device for testing if breast milk has spoiled comprises a vessel for holding the sample of breast milk, and a cap for closing the vessel which already contains the detecting agent, which upon mixing enters the vessel.

In an additional aspect, a device for testing if breast milk has spoiled comprises a vessel for holding the sample of breast milk, and a cap for closing the vessel which contains one crushable ampoule containing the detecting agent, which upon breaking enters the vessel.

In certain embodiments, the crushable ampoule is composed of glass, polymer, metal, ceramic or combinations thereof.

In other embodiments, the vessel is a vial, cup, mug, chamber, container, beaker, syringe, goblet, reservoir composed of glass, polymer, metal, ceramic or combinations thereof. In some embodiments, the vessel said is marked with a graduated scale so as to add a specific, known, volume of milk.

In additional embodiments the cap is composed of glass, polymer, metal, ceramic or combinations thereof. In some examples, the cap is a screw cap, twist, zip-tie, pinch, stopper, or snap cap.

In certain examples the sample of breast milk is a sample of mammalian breast milk. In other examples the sample of mammalian breast milk is primate, bovine, ovine, caprine, equine, porcine, murine, feline, or canine. In one example, the sample is human milk.

In additional embodiments, the device further comprises a medicament, colorant, flavoring, scent, fibrous additive, antioxidant, thickener, or plasticizer.

In some embodiments, a method comprising the steps of determining if a sample of breast milk has spoiled is used. In additional embodiments, the method comprises the steps of determining if a sample of breast milk has spoiled using 1000 to 500 mL of breast milk. In other embodiments, the method comprises the steps of determining if a sample of breast milk has spoiled using 500 to 100 mL of breast milk. In certain examples, the method comprises the steps of determining if a sample of breast milk has spoiled using 100 to 50 mL of breast milk. In some examples, the method comprises the steps of determining if a sample of breast milk has spoiled using 50 to 10 mL of breast milk. In other examples, the method comprises the steps of determining if a sample of breast milk has spoiled using 10 to 1 mL of breast milk. In certain embodiments, the method comprises the steps of determining if a sample of breast milk has spoiled using 1 to 0.1 mL of breast milk. In some embodiments, the method comprises the steps of determining if a sample of breast milk has spoiled using 0.1 to 0.01 mL of breast milk. In additional embodiments, the method comprises the steps of determining if a sample of breast milk has spoiled using 0.01 to 0.001 mL of breast milk. In other embodiments, the method comprises the steps of determining if a sample of breast milk has spoiled using 0.001 to 0.0001 mL of breast milk.

In certain embodiments the method of testing comprises the steps of first adding said base to said milk sample to give a mixture, and second adding said detecting agent to said mixture. In other embodiments the method of testing comprises the steps of first adding said detecting agent to said milk sample to give a mixture, and then adding said base to said mixture. In some examples the method of testing comprises the step of first concurrently adding said detecting agent and said base to said milk sample. In certain examples the method of testing comprises the step of only adding said detecting agent to said milk sample.

In some examples the method of testing comprises the steps of first passing said milk sample through a resin or filter treated with said base, and then exposing said sample to said detecting agent, affording a signal. In other examples the method of testing comprises the steps of first passing said milk sample through a resin or filter treated with said base and said detecting agent, to afford a signal. In further examples the method of testing comprises the steps of first passing said milk sample through a resin or filter which is a basic resin, and then exposing said sample to said detecting agent, affording a signal. In certain embodiments the method of testing comprises the steps of first passing said milk sample through a resin or filter treated with said detecting agent affording a signal. In some embodiments the method of testing comprises the steps of first passing said milk sample through a resin or filter to remove particulates.

In another aspect a concentration-type assay test device for determining if a sample of breast milk has excess endotoxin load is provided. In certain embodiments the concentration assay for determining if a sample of breast milk has spoiled comprises a detection agent alone. In other embodiments the concentration assay for determining if a sample of breast milk has spoiled, comprises a detection agent in combination with a dye to aid in visualization. In some embodiments the concentration is determined by visual inspection, application of a light source, or application of an electrochemical source.

In one embodiment the detecting agent is Limulus amoebocyte lysate.

In some embodiments the detecting agent is in contact with paper, polymer, glass, metal, ceramic, metal oxide, graphite, aqueous solution, alcoholic solution, organic solution, film, porous film, filter, microparticle, nanoparticle, or nanotube. In certain embodiments the detecting agent and dye are in contact with paper, polymer, glass, metal, ceramic, metal oxide, graphite, aqueous solution, alcoholic solution, organic solution, film, porous film, filter, microparticle, nanoparticle, or nanotube. In some examples the contact is achieved through absorption, adsorption, and/or covalent linkage. In further examples the detecting agent is immobilized chemically or by a gel matrix. In other examples the detecting agent is a solid, dissolved in an aqueous solution, alcoholic, aqueous-alcoholic solution, organic solution, or neat. In certain embodiments the aqueous or aqueous-alcoholic solution has an osmotic pressure of about 100 mOs/kg to about 700 mOs/kg. In other embodiments the aqueous or aqueous-alcoholic solution has an osmotic pressure of about 200 mOs/kg to about 400 mOs/kg. In some embodiments the aqueous or aqueous-alcoholic solution has a pH of about 1 to about 12 or higher. In further embodiments the aqueous or aqueous-alcoholic solution has a pH of about 5 to about 8. In some examples the aqueous or aqueous-alcoholic solution has a pH of about 6 to about 7. In certain examples the aqueous or aqueous-alcoholic solution has a pH of about 1 to about 12 following contact with a sample of breast milk. In further examples the aqueous or aqueous-alcoholic solution has a pH of about 5 to about 8 following contact with a sample of breast milk. In other examples the aqueous or aqueous-alcoholic solution has a pH of about 6 to about 7 following contact with a sample of breast milk.

In some embodiments the detecting agent is a molecule, macromolecule, or polymer. In another embodiment the detecting molecule, macromolecule, or polymer is a pH indicator, dye, redox indicator, or metabolic indicator. In some embodiments the visualization dye is selected from the comprising: litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino) azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier' s blue, trinitrobenzoic acid, Congo red, Azolitmin, Neutral red, Cresol Red, Alizarine Yellow R and salts thereof. In further embodiments more than one detecting agent and/or dye is present. In an additional embodiment the detecting agent is a solid and said dye is in solution.

In another aspect, a device for testing if breast milk has endotoxins comprises a vessel for holding the sample of breast milk which already contains both the detecting agent and dye, and a cap for closing the vessel.

In an additional aspect, a device for testing if breast milk has endotoxins comprises a vessel for holding the sample of breast milk which already contains the detecting agent and a crushable ampoule containing the dye, and a cap for closing the vessel.

In another aspect, a device for testing if breast milk has endotoxins comprises a vessel for holding the sample of breast milk which already contains the detecting agent, and a cap for closing the vessel which already contains the dye, which upon mixing enters the vessel.

In an additional aspect a device for testing if breast milk has endotoxins comprises a vessel for holding the sample of breast milk which already contains the detecting agent, and a cap for closing the vessel which contains one crushable ampoule containing the dye, which upon breaking enters the vessel.

In another aspect, a device for testing if breast milk has endotoxins comprises a vessel for holding the sample of breast milk which already contains the dye and a crushable ampoule containing the detecting agent, and a cap for closing the vessel.

In an additional aspect a device for testing if breast milk has endotoxins comprises a vessel for holding the sample of breast milk containing a crushable ampoule containing both the detecting agent and the dye and a cap for closing the vessel.

In another aspect, a device for testing if breast milk has endotoxins comprises a vessel for holding the sample of breast milk containing two crushable ampoules one containing the detecting agent and the other containing the dye, and a cap for closing the vessel.

In an additional aspect, a device for testing if breast milk has endotoxins comprises a vessel for holding the sample of breast milk containing a crushable ampoule containing the detecting agent, and a cap for closing the vessel which already contains the dye, which upon mixing enters the vessel.

In another aspect, a device for testing if breast milk has endotoxins comprises a vessel for holding the sample of breast milk containing a crushable ampoule containing the detecting agent, and a cap for closing the vessel which contains one crushable ampoule containing the dye, which upon breaking enters the vessel.

In an additional aspect a device for testing if breast milk has endotoxins comprises a vessel for holding the sample of breast milk which already contains the dye, and a cap for closing the vessel which already contains the detecting agent, which upon mixing enters the vessel.

In another aspect, a device for testing if breast milk has endotoxins comprises a vessel for holding the sample of breast milk which already contains the dye, and a cap for closing the vessel which contains one crushable ampoule containing the detecting agent, which upon breaking enters the vessel.

In an additional aspect, a device for testing if breast milk has endotoxins comprises a vessel for holding the sample of breast milk which contains a crushable ampoule containing the dye, and a cap for closing the vessel which already contains the detecting agent, which upon mixing enters the vessel.

In another aspect, a device for testing if breast milk has endotoxins comprises a vessel for holding the sample of breast milk which contains a crushable ampoule containing the dye, and a cap for closing the vessel which contains one crushable ampoule containing the detecting agent, which upon breaking enters the vessel.

In an additional aspect, a device for testing if breast milk has endotoxins comprises a vessel for holding the sample of breast milk, and a cap for closing the vessel which already contains both the detecting agent and dye, which upon mixing enter the vessel.

In another aspect, a device for testing if breast milk has endotoxins comprises a vessel for holding the sample of breast milk, and a cap for closing the vessel which already contains the detecting agent and a crushable ampoule containing the dye, which upon breaking and mixing enter the vessel.

In an additional aspect, a device for testing if breast milk has endotoxins comprises a vessel for holding the sample of breast milk, and a cap for closing the vessel which already contains the dye and a crushable ampoule containing the detecting agent, which upon breaking and mixing enter the vessel.

In another aspect, a device for testing if breast milk has endotoxins comprises a vessel for holding the sample of breast milk, and a cap for closing the vessel which contains a crushable ampoule containing both the detecting agent and the dye, which upon breaking enter the vessel.

In an additional aspect, a device for testing if breast milk has endotoxins comprises a vessel for holding the sample of breast milk, and a cap for closing the vessel which contains two crushable ampoules one containing the detecting agent and the other containing the dye, which upon breaking enter the vessel.

In another aspect, a device for testing if breast milk has endotoxins comprises a vessel for holding the sample of breast milk which already contains the detecting agent, and a cap for closing the vessel.

In an additional aspect, a device for testing if breast milk has endotoxins comprises a vessel for holding the sample of breast milk containing a crushable ampoule containing the detecting agent, and a cap for closing the vessel.

In another aspect, a device for testing if breast milk has endotoxins comprises a vessel for holding the sample of breast milk, and a cap for closing the vessel which already contains the detecting agent, which upon mixing enters the vessel.

In an additional aspect, a device for testing if breast milk has endotoxins comprises a vessel for holding the sample of breast milk, and a cap for closing the vessel which contains one crushable ampoule containing the detecting agent, which upon breaking enters the vessel.

In certain embodiments the crushable ampoule is composed of glass, polymer, metal, ceramic or combinations thereof. In other embodiments the vessel is a vial, cup, mug, chamber, container, beaker, syringe, goblet, reservoir composed of glass, polymer, metal, ceramic or combinations thereof. In some examples the vessel is marked with a graduated scale so as to add a specific, known, volume of milk. In further embodiments the cap is composed of glass, polymer, metal, ceramic or combinations thereof. In certain embodiments the cap is a screw cap, twist, zip-tie, pinch, stopper, or snap cap.

In some embodiments the sample of breast milk is a sample of mammalian breast milk. In further embodiments the sample of mammalian breast milk is primate, bovine, ovine, caprine, equine, porcine, murine, feline, or canine. In additional embodiments the breast milk sample is human.

In some examples the device further comprises a medicament, colorant, flavoring, scent, fibrous additive, antioxidant, thickener, or plasticizer.

In some examples a method comprising the step of determining if a sample of breast milk has spoiled using the device is described. In further examples a method comprises the step of determining if a sample of breast milk has spoiled using the device whereby 1000 to 500 mL of breast milk are used. In additional examples a method comprises the step of determining if a sample of breast milk has spoiled using the device whereby 500 to 100 mL of breast milk are used. In some embodiments a method comprises the step of determining if a sample of breast milk has spoiled using the device whereby 100 to 50 mL of breast milk are used. In certain examples a method comprises the step of determining if a sample of breast milk has spoiled using the device whereby 50 to 10 mL of breast milk are used. In some embodiments a method comprises the step of determining if a sample of breast milk has spoiled using the device whereby 10 to 1 mL of breast milk are used. In other examples a method comprises the step of determining if a sample of breast milk has spoiled using the device whereby 1 to 0.1 mL of breast milk are used. In another embodiment a method comprises the step of determining if a sample of breast milk has spoiled using the device whereby 0.1 to 0.01 mL of breast milk are used. In an additional embodiment a method comprises the step of determining if a sample of breast milk has spoiled using the device whereby 0.01 to 0.001 mL of breast milk are used. In other examples a method comprises the step of determining if a sample of breast milk has spoiled using the device whereby 0.001 to 0.0001 mL of breast milk are used.

In certain embodiments the method of testing which comprises the steps of first adding said dye to said milk sample to give a mixture, and second adding said detecting agent to said mixture is provided.

In some embodiments the method of testing which comprises the steps of first adding said detecting agent to said milk sample to give a mixture, and then adding said dye to said mixture is described.

In other embodiments the method of testing which comprises the step of first concurrently adding said detecting agent and said dye to said milk sample is disclosed.

In another embodiment the method of testing which comprises the step of only adding said detecting agent to said milk sample is used.

In an additional embodiment the method of testing which comprises the steps of first passing said milk sample through a resin or filter treated with said dye, and then exposing said sample to said detecting agent, affording a signal is disclosed.

In some examples the method of testing which comprises the steps of first passing said milk sample through a resin or filter treated with said dye and said detecting agent, to afford a signal is described.

In further examples the method of testing which comprises the steps of first passing said milk sample through a resin or filter treated with said detecting agent affording a signal is provided.

In other examples the method of testing which comprises the steps of first passing said milk sample through a resin or filter to remove particulates is described.

In certain embodiments the kit may be prepared by using a sterilization method of said device. In some embodiments the sterilization of the device utilizes visible light irradiation, ultraviolet light, electron-beam radiation, gamma-radiation, chemical techniques, physical techniques, or combinations thereof. In other embodiments the sterilization of the device utilizes chemical techniques; and said chemical techniques comprise exposure to ethylene oxide or hydrogen peroxide vapor. In further embodiments the sterilization method of the device utilizes physical techniques; and said physical techniques comprise moist heating, dry heating, retort canning, or filtration. In another embodiment the sterilization of the device utilizes electron-beam radiation or gamma-radiation; and the amount of said radiation is between about 2 and about 40 kGy. In certain examples the sterilization of the device utilizes electron-beam radiation or gamma-radiation; and the amount of said radiation is between about 3 and about 20 kGy. In other examples the sterilization of the device utilizes electron-beam radiation or gamma-radiation; and the amount of said radiation is between about 5 and about 12 kGy. In additional examples the sterilizing radiation is applied more than once. In further examples the sterilization of the device is conducted below about 150° C. In certain examples the sterilization of the device is conducted below about 100° C. In additional embodiments the sterilization of the device is conducted below about 50° C. In some embodiments the sterilization of the device is conducted below about 30° C. In other embodiments the sterilization of the device is conducted below about 20° C. In certain embodiments the sterilization of the device is conducted below about 10° C. In additional embodiments the sterilization of the device is conducted below about 0° C.

In some examples the sample of breast milk is from a primate, bovine, ovine, caprine, equine, porcine, murine, feline, or canine. In other examples the described method is for testing sample of breast milk is from a human.

In additional embodiments the procedure further comprises the step of monitoring the breast milk for spoilage over a period of time. In certain embodiments the monitoring period of time is about six months to about one year. In other examples the monitoring period of time is about six months. In some examples the monitoring period of time is about one year. In an additional aspect a kit may comprise instructions for use of the device. In further aspects the kit may contain one or more devices and an instruction manual. In additional embodiments the kit may comprise one or more devices, a delivery system for adding the sample to the device and an instruction manual. In another embodiment the kit comprises one or more devices, a delivery system, an instruction manual and a logbook for recording the history of readings. In some embodiments the kit comprises one or more devices, a delivery system, an instruction manual and a chart for plotting the history of readings. In further embodiments the kit comprises a delivery system, an instruction manual, and an instruction booklet on how to record the history of readings on a secured on-line website.

In some embodiments the delivery system is a syringe, a spoon, a pipette, an eye dropper, teaspoon, tablespoon, or a capillary tube.

In additional embodiments the kit further comprises a desiccant or an antioxidant. In some examples the antioxidant is selected from the group consisting of sodium metabisulfite, citric acid, and ascorbic acid.

In another embodiment the kit further comprises the device stored in an inert atmosphere.

In some examples the kit has a sterility assurance level of at least about 10⁻³. In other examples the kit has a sterility assurance level of at least about 10⁻⁶.

In certain examples the kit further comprises a moisture-barrier element with a moisture vapor transmission rate (MVTR) less than or equal to about 0.15 gram per 100 square inches per day. In additional examples the kit further comprising a moisture-barrier element with a moisture vapor transmission rate (MVTR) less than or equal to about 0.02 gram per 100 square inches per day.

In some embodiments the moisture-barrier element comprises the device. In other embodiments the moisture-barrier element comprises the detecting agent. In further embodiments the moisture-barrier element comprises the base.

In an additional example the kit is protected from light.

In certain embodiments the kit is disposable. In other embodiments the kit is recyclable.

In certain aspects the kit can be used in the home, workplace, clinic, outpatient office, hospital, train, airplane, boat, car, and outdoors.

In some embodiments a concentration-type assay test device is used for determining if a sample of breast milk has spoiled comprising a detecting agent and a base where an incomplete acid-base reaction occurs between the base and the acid in breast milk such that the detecting agent changes.

In other embodiments a concentration-type assay test device is used for determining if a sample of breast milk has spoiled, comprising a detecting agent and a base where an incomplete acid-base reaction occurs between the base and the lactic acid in breast milk such that the detecting agent changes.

In further embodiments the method whereby an incomplete acid-base reaction occurs between the base and the acid in breast milk such that the detecting agent changes color is disclosed.

In additional embodiments the method whereby an incomplete acid-base reaction occurs between the base and the lactic acid in breast milk such that the detecting agent changes color in provided.

In certain embodiments the method whereby a metabolic detecting agent is used to determine the amount of active bacteria present in the milk sample is described.

In an additional aspect an assay test device for determining the fat or caloric content of breast milk using the timing or speed with which a sample of milk flows across a surface as an indicator of the fat or caloric content of the breast milk is disclosed.

In certain examples an assay test device for determining the fat or caloric content of breast milk using the timing at which a sample of milk resides at an opening or ridge as an indicator of the fat or caloric content of the breast milk is provided.

In some examples an assay test device for determining the fat or caloric content of breast milk using the timing at which a sample of milk interacts with a polymer surface and that interaction leads to an indicator of the fat or caloric content of the breast milk is disclosed.

In certain embodiments the concentration is determined by visual inspection, application of a light source, application of an electrochemical source, application of a sound source, or application of a flow counter.

In some examples the detecting surface is a polymer including but not limited to Teflon, polystyrene, modified polystyrene, polypropylene, polyurethane, ethylene vinyl alcohol, (E/VAL), fluoroplastics, (PTFE), (FEP, PFA, CTFE, ECTFE, ETFE, polyacrylates, (Acrylic). polybutadiene, (PBD), polybutylene, (PB), polyethylene, (PE), polyethylenechlorinates, (PEC), polymethylpentene, (PMP), polypropylene, (PP), polyvinylchloride, (PVC), polyvinylidene chloride, (PVDC), acrylonitrile butadiene styrene, (ABS), Polyamide, (PA), (Nylon), polyamide-imide, (PAI), polyaryletherketone, (PAEK), (Ketone), polycarbonate, (PC), polyektone, (PK), polyester, polyetheretherketone, (PEEK), polyetherimide, (PEI), polyethersulfone, (PES), polyimide, (PI), polyphenylene oxide, (PPO), polyphenylene sulfide, (PPS), polyphthalamide, (PTA), polysulfone, (PSU), allyl resin, (Allyl), melamine formaldehyde, (MF), phenol-formaldehyde plastic, (PF), (Phenolic), polyester, polyimide, (PI), polydimethylsiloxane (PDMS), silicone, (SI).

In other embodiments the surface is a metal, metal oxide, nonmetal oxide, ceramic, including but not limited to TiO2, SiO2, titanium, stainless steel, gold, platinum, pladium, silver.

In additional embodiments the surface is a metal surface coated with a small molecule or polymer wherein, for example, the metal is gold and the small molecule is a dodecane thiol.

In some embodiments the surface is composed of two or more materials including but not limited to polymers, metals, metal oxide, ceramics, and nonmetal oxides.

In certain examples the surface is shaped into a channel, groove, tube, or other geometric manipulation.

In some examples the milk sample has an osmotic pressure of about 100 mOs/kg to about 700 mOs/kg. In other examples the milk sample has an osmotic pressure of about 200 mOs/kg to about 400 mOs/kg. In another example the milk sample has a pH of about 1 to about 12 or higher. In an additional example the milk sample has a pH of about 5 to about 8. In certain embodiments the milk sample has a pH of about 6 to about 7. In some embodiments the milk sample has a pH of about 1 to about 12 after the measurement. In certain embodiments the milk sample has a pH of about 5 to about 8 following contact with the device. In further embodiments the milk sample has a pH of about 6 to about 7 following contact with device.

In an additional embodiment a dye or more than two dyes are (is) added to the milk sample to aid in visualization wherein the dye is selected from but not limited to the group consisting of: litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino) azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R, FD&C Red 3, FD&C Red 40, FD&C Yellow 5, FD&C Yellow 6, FD&C Blue 1, FD&C Blue 2, FD&C Green 3, Caramel Coloring, Annatto, Chlorella, Cochineal, Beet Juice, Saffron, Paprika, Tumeric, Anthrocyanin, Chlorophyll, beta-Carotene, B-Apo-8′-Carotenal, Canthaxanthin, Carrot Oil, Cottonseed Flour, Ferrous Gluconate, Grape Extract, Riboflavin, Carminic Acid, Titanium Dioxide, and salts thereof.

In another embodiment a gradient of or two color changes are observed.

In an additional embodiment a molecule, macromolecule, or polymer is added to the milk where a redox active species increases the conductivity of the milk sample to aid in detection and subsequent determination of the fat or calorie content. In a further embodiment the molecularspecies is selected from the group consisting of but not limited to NaCl, KCl, NaBr, NaI, KBr, KI, ferrocene; tris(2,2′-bipyridine)ruthenium (II); and tris(2,2′-bipyridine)osmium (II), derivatizied ferrocene, methyl violagen, polythiophene, polyanaline, polypyrrole, ruthenium trisbypridine, transitional metal complex, and conducting polymer.

In some embodiments the testing procedure for obtaining the fat or caloric content of breast milk comprises the steps of adding the milk sample to a cartridge and inserting this sample into a detector followed by performing a measurement on the sample.

In some embodiments the cartridge and/or the counter may be disposable, recyclable, or reusable. In additional embodiments the vessel or cartridge is composed of glass, polymer, metal, ceramic or combinations thereof. In further embodiments the detector contains a vessel is a vial, cup, mug, chamber, container, beaker, syringe, goblet, reservoir composed of glass, polymer, metal, ceramic or combinations thereof. In certain embodiments the vessel or cartridge is marked with a graduated scale so as to add a specific, known, volume of milk.

In some embodiments the sample of breast milk is a sample of mammalian breast milk. In other embodiments the sample of mammalian breast milk is primate, bovine, ovine, caprine, equine, porcine, murine, feline, or canine. In further embodiments the sample of breast milk is human.

In some examples the device additionally comprises a medicament, colorant, flavoring, scent, fibrous additive, antioxidant, thickener, or plasticizer.

In further embodiments the steps of determining the fat or caloric content of breast milk using the device is described. In some examples the steps of determining the fat or caloric content of breast milk using the device is provided. In additional embodiments the steps of determining the fat or caloric content of breast milk using the device whereby 500 to 100 mL of breast milk are used is disclosed. In some embodiments the steps of determining the fat or caloric content of breast milk using the device whereby 100-50 mL of breast milk is provided. In additional embodiments a method comprising the steps of determining the fat or caloric content of breast milk using the device whereby 50-10 mL of breast milk are used is described. In another embodiment the steps of determining the fat or caloric content of breast milk using the device whereby 10-1 mL of breast milk are used is disclosed. In some examples the steps of determining the fat or caloric content of breast milk using the device whereby 1-0.1 mL of breast milk are used is provided. In further examples the steps of determining the fat or caloric content of breast milk using the device whereby 0.1-0.01 mL of breast milk are used is described. In certain examples the steps of determining the fat or caloric content of breast milk whereby 0.01-0.001 mL of breast milk is provided. In another example the steps of determining the fat or caloric content of breast milk using the device whereby 0.001-0.0001 mL of breast milk are used is disclosed.

In certain embodiments a method of testing is used which comprises the steps of first passing said milk sample through a resin or filter, and then exposing said sample to surface for subsequent detection and determination of the fat or caloric content of the sample.

In other embodiments sterilization of the device is conducted utilizing visible light irradiation, ultraviolet light, electron-beam radiation, gamma-radiation, chemical techniques, physical techniques, or combinations thereof. In some examples the sterilization of said device utilizes chemical techniques; and said chemical techniques comprise exposure to ethylene oxide or hydrogen peroxide vapor. In other examples the sterilization of the device utilizes physical techniques; and the physical techniques comprise moist heating, dry heating, retort and hot-fill canning, or filtration. In certain examples the sterilization of the device utilizes electron-beam radiation or gamma-radiation; and the amount of said radiation is between about 2 and about 40 kGy. In an additional example the sterilization of the device utilizes electron-beam radiation or gamma-radiation; and the amount of said radiation is between about 3 and about 20 kGy. In another example the sterilization of said device utilizes electron-beam radiation or gamma-radiation; and the amount of said radiation is between about 5 and about 12 kGy. In other embodiments the radiation is applied once or more than once. In some embodiments the amount of the radiation is between about 5 and about 40 kGy.

In certain embodiments sterilization of the device is conducted below about 150° C. In additional embodiments sterilization of the device is conducted below about 100° C. In another embodiment sterilization of the device is conducted below about 50° C. In further embodiments sterilization of the device is conducted below about 30° C. In other examples sterilization of the device is conducted below about 20° C. In certain examples sterilization of the device is conducted below about 10° C. In another example sterilization of the device is conducted below about 0° C.

In an additional example the sample of breast milk is from a primate, bovine, ovine, caprine, equine, porcine, murine, feline, or canine. In another example the sample of breast milk is from a human.

A further example comprises the steps of monitoring the fat or caloric content of milk over a period of time. In another example the monitoring period of time is about six months to about one year. In an additional example the monitoring period of time is about one to six months. In certain examples the monitoring period of time is less than one month.

In another embodiment the mother records her caloric measurements along with time since last eating and time of day in a supplied logbook. In some embodiments the mother records her caloric measurements along with time since last eating and time of day on a supplied graph/plot. In certain embodiments the mother records her caloric measurements along with time since last eating and time of day in a website database.

In an additional example the method further comprises the steps of affecting or monitoring the intake diet of a newborn or infant for a period of time based on the mothers diet. This method further comprises the steps of (i) measuring the fat content of breast milk; (ii) feeding said newborn or infant; (iii) optionally measuring the fat content of breast milk; (iv) optionally logging her measurement to determine ideal times to feed; (v) optionally feeding or first changing the diet of and feeding said newborn or infant; (vi) optionally repeating (iii) and/or (iv) and/or (v or vi).

In another embodiment a kit is described which comprises instructions for use thereof. In an additional embodiment a kit is disclosed which comprises one or more devices and an instruction manual. In certain embodiments a kit is described which comprises one or more devices, a delivery system, and an instruction manual. In some examples a kit is disclosed which comprises one or more devices, a delivery system, an instruction manual and a logbook for recording the history of readings. In further examples a kit is described which comprises one or more devices, a delivery system, an instruction manual and a chart for plotting the history of readings. In some examples a kit is described which comprises one or more devices, a delivery system, an instruction manual, and an instruction booklet on how to record the history of readings on a secured on-line website.

In another example the delivery system is a syringe, a spoon, a pipette, an eye dropper, teaspoon, tablespoon, or a capillary tube.

In some examples the kit further comprises a desiccant or an antioxidant. In certain the antioxidant is selected from the group consisting of sodium metabisulfite, citric acid, and ascorbic acid.

In other examples the kit further comprises the device in an inert atmosphere.

In some embodiments the kit has a sterility assurance level of at least about 10⁻³. In other embodiments the kit has a sterility assurance level of at least about 10⁻⁶.

In further embodiments the kit includes a moisture-barrier element with a moisture vapor transmission rate (MVTR) less than or equal to about 0.15 gram per 100 square inches per day. In an additional embodiment the kit includes a moisture-barrier element with a moisture vapor transmission rate (MVTR) less than or equal to about 0.02 gram per 100 square inches per day. In some embodiments the moisture-barrier element comprises the device. In certain embodiments the moisture-barrier element comprises the cartridge. In other embodiments the moisture-barrier element comprises the counter.

In further examples the kit is protected from light.

In an additional example the kit is disposable.

In another example the kit is recyclable.

In certain examples the kit can be used in the home, workplace, clinic, outpatient office, milk bank, hospital, train, airplane, boat, car, and outdoors.

In some embodiments the surface dependent concentration-type assay test device for determining the fat or caloric content of breast milk where a surface interacts with the milk such that surface affects the rate at which the milk travels upon it based on the fat content of the milk sample is disclosed.

In other embodiments an assay test device for determining the fat or caloric content of breast milk using the timing at which a sample of milk resides at an opening or ridge as an indicator of the fat or caloric content of the breast milk is provided.

In additional embodiments an assay test device for determining the fat or caloric content of breast milk using the timing at which a sample of milk interacts with a polymer surface and that interaction leads to an indicator of the fat or caloric content of the breast milk is described.

In further embodiments the method whereby a surface(s) interacts with the milk such that the detection of the fat or caloric content of milk is possible because the detection mode is dependent on the fat content of the milk sample is disclosed.

In an additional aspect a concentration-type assay test device for determining if a sample of breast milk has a metal, comprising a detecting agent and/or an enzyme and/or a substrate is disclosed. In further embodiments the concentration is determined by visual inspection, application of a light source, or application of an electrochemical source.

In some examples the detecting agent is in contact with paper, polymer, glass, metal, ceramic, metal oxide, graphite, aqueous solution, alcoholic solution, organic solution, film, porous film, filter, microparticle, nanoparticle, or nanotube.

In certain examples the detecting agent and enzyme are in contact with paper, polymer, glass, metal, ceramic, metal oxide, graphite, aqueous solution, alcoholic solution, organic solution, film, porous film, filter, microparticle, nanoparticle, or nanotube.

In further examples the contact is by absorption, adsorption, and/or covalent linkage. In certain examples the detecting agent is immobilized chemically or by a gel matrix.

In other examples the detecting agent is a solid, dissolved in an aqueous solution, alcoholic, aqueous-alcoholic solution, organic solution, or neat. In another embodiment the aqueous or aqueous-alcoholic solution has an osmotic pressure of about 100 mOs/kg to about 700 mOs/kg. In an additional embodiment the aqueous or aqueous-alcoholic solution has an osmotic pressure of about 200 mOs/kg to about 400 mOs/kg. In some examples the aqueous or aqueous-alcoholic solution has a pH of about 1 to about 12 or higher. In other examples the aqueous or aqueous-alcoholic solution has a pH of about 5 to about 8. In certain examples the aqueous or aqueous-alcoholic solution has a pH of about 6 to about 7. In further examples the aqueous or aqueous-alcoholic solution has a pH of about 1 to about 12 following contact with a sample of breast milk. In another example the aqueous or aqueous-alcoholic solution has a pH of about 5 to about 8 following contact with a sample of breast milk. In other embodiments the aqueous or aqueous-alcoholic solution has a pH of about 5 to about 8 following contact with a sample of breast milk.

In an additional embodiment the detecting agent is a molecule, macromolecule, or polymer.

In a further embodiment the molecule, macromolecule, or polymer is a pH indicator or dye.

In an additional example the detecting agent is selected from but not limited to the group consisting of: litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino) azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R and salts thereof. In another example more than one detection agent is present. In an additional example a gradient of two, three, four, or more color changes are observed.

In some embodiments the molecule, macromolecule, or polymer is a redox active species.

In certain embodiments the detecting agent is selected from the group consisting of ferrocene; tris(2,2′-bipyridine)ruthenium (II); and tris(2,2′-bipyridine)osmium (II), derivatizied ferrocene, methyl violagen, polythiophene, polyanaline, polypyrrole, ruthenium trisbypridine, transitional metal complex, and conducting polymer.

In other embodiments the enzyme is a solid, dissolved in an aqueous solution, buffered solution, alcoholic solution, aqueous-alcoholic solution, or neat. In another embodiment the enzyme is from but not to the following list: mercuric reductase, 1-lactate dehydrogenase, invertase, δ-aminolevulinate dehydrogenase, pyruvate dehydrogenase, alkaline phosphatase, horseradish peroxidase, caspase, and urease, or an oxidoreductase, transferase, hydrolase, lyase, isomerase, or ligase, or a combination of two or more different enzymes.

In an additional embodiment the substrate may be selected from the following list but not limited to: urea, NADPH, lactate, pyruvate, sucrose, δ-aminolevulinate acid , para-nitrophenyl phosphate, 2-2′-azino-di-(3-ethylbenz-thiazoline sulfonic acid), o-phenylenediamine, tetramethylbenzidine, or some variation of a dye bound to the tetrapeptide sequence aspartic acid-glutamic acid-valine-aspartic acid.

In other embodiments the metal is mercury, inorganic mercury, organic mercury, mercury chloride, mercury bromide, mercury acetate, mercury iodide, lead, lead chloride, lead acetate, lead bromide, lead iodide, antimony (Sb), arsenic (As), cadmium (Cd), calcium(Ca), chlorine (Cl), chromium (Cr), cobalt (Co), copper (Cu), fluorine (F), iodine (I), iron (Fe), lead (Pb), magnesium (Mg), manganese (Mn), mercury (Hg), molybdenum (Mo), nickel (Ni), phosphorus (P), potassium (K), selenium (Se), sodium (Na), tin (Sn), vanadium (V), and zinc (Zn).

In some examples the detecting agent precipitates to give a signal.

In one example the detecting agent is bromothymol blue.

In another example the detecting agent is a combination of bromothymol blue and another detecting agent such as thymol blue, methyl red, and/or phenolphthalein.

In some embodiments the solution of detecting agent is separate from a solution of enzyme and a solution of substrate. In certain embodiments the solution of detecting agent is separate from a solution of enzyme and a solid substrate. In another example the solution of detecting agent is separate from an enzyme as a solid and a solution of a substrate. In an additional example the solution of detecting agent is separate from enzyme as a solid and a substrate as a solid. In certain examples the detecting agent is a solid and separate from a solution of enzyme and a solution of a substrate. In further examples the detecting agent is a solid and is separated from an enzyme as a solid and a solution of a substrate. In some examples the detecting agent is a solid and is separated from a solution of an enzyme and a substrate as a solid.

In an additional embodiment the solution of detecting agent can be a mixture of both (enzyme and dye) or two or more different solutions (one enzyme and one dye and one substrate).

In one example the enzyme is urease.

In another example the detecting agent is bromothymol blue and said enzyme is urease.

In a further example the detecting agent is bromothymol blue, said substrate is urea, and said enzyme is urease.

In another aspect, a device for testing if breast milk has a metal comprises a vessel for holding the sample of breast milk which already contains the detecting agent, enzyme, and substrate, and a cap for closing the vessel.

In an additional aspect, a device for testing if breast milk has a metal comprises a vessel for holding the sample of breast milk which already contains the detecting agent and the enzyme and a crushable ampoule containing the substrate, and a cap for closing the vessel.

In another aspect, a device for testing if breast milk has a metal comprises a vessel for holding the sample of breast milk which already contains the detecting agent and the substrate and a crushable ampoule containing the enzyme and a cap for closing the vessel.

In an additional aspect, a device for testing if breast milk has a metal comprises a vessel for holding the sample of breast milk which already contains the detecting agent and two crushable ampoules one containing the substrate and one containing the enzyme, and a cap for closing the vessel.

In another aspect, a device for testing if breast milk has a metal comprises a vessel for holding the sample of breast milk which already contains both the detecting agent and the enzyme, and a cap for closing the vessel which already contains the substrate, which upon mixing enters the vessel.

In an additional aspect, a device for testing if breast milk has a metal comprises a vessel for holding the sample of breast milk which already contains both the detecting agent and the enzyme, and a cap for closing the vessel which contains one crushable ampoule containing the substrate, which upon breaking enters the vessel.

In another aspect, a device for testing if breast milk has a metal comprises a vessel for holding the sample of breast milk which already contains the detecting agent and one crushable ampoule containing the enzyme, and a cap for closing the vessel which already contains the substrate, which upon mixing enters the vessel.

In an additional aspect, a device for testing if breast milk has a metal comprises a vessel for holding the sample of breast milk which already contains the detecting agent and one crushable ampoule containing the enzyme, and a cap for closing the vessel which contains one crushable ampoule containing the substrate, which upon mixing enters the vessel.

In another aspect, a device for testing if breast milk has a metal comprises a vessel for holding the sample of breast milk which already contains both the detecting agent and the substrate, and a cap for closing the vessel which already contains the enzyme, which upon mixing enters the vessel.

In an additional aspect, a device for testing if breast milk has a metal comprises a vessel for holding the sample of breast milk which already contains both the detecting agent and the substrate, and a cap for closing the vessel which contains one crushable ampoule containing the enzyme, which upon breaking enters the vessel.

In another aspect, a device for testing if breast milk has a metal comprises a vessel for holding the sample of breast milk which already contains the detecting agent and one crushable ampoule containing the substrate, and a cap for closing the vessel which already contains the enzyme, which upon mixing enters the vessel.

In an additional aspect, a device for testing if breast milk has a metal comprises a vessel for holding the sample of breast milk which already contains the detecting agent and one crushable ampoule containing the substrate, and a cap for closing the vessel which contains one crushable ampoule containing the enzyme, which upon breaking enters the vessel.

In another aspect, a device for testing if breast milk has a metal comprises a vessel for holding the sample of breast milk which already contains the detecting agent, and a cap for closing the vessel which already contains both the enzyme and the substrate, which upon mixing enters the vessel.

In an additional aspect, a device for testing if breast milk has a metal comprises a vessel for holding the sample of breast milk which already contains the detecting agent, and a cap for closing the vessel which already contains the enzyme and one crushable ampoule containing the substrate, which upon breaking and mixing enter the vessel.

In another aspect, a device for testing if breast milk has a metal comprises a vessel for holding the sample of breast milk which already contains the detecting agent, and a cap for closing the vessel which already contains the substrate and one crushable ampoule containing the enzyme, which upon breaking and mixing enter the vessel.

In an additional aspect, a device for testing if breast milk has a metal comprises a vessel for holding the sample of breast milk which already contains the detecting agent, and a cap for closing the vessel which contains two crushable ampoules, one containing the enzyme and one containing the substrate, which upon breaking enter the vessel.

In another aspect, a device for testing if breast milk has a metal comprises a vessel for holding the sample of breast milk which already contains the enzyme and substrate and one crushable ampoule containing the detecting agent, and a cap for closing the vessel.

In an additional aspect, a device for testing if breast milk has a metal comprises a vessel for holding the sample of breast milk which already contains the enzyme and two crushable ampoule one containing the detecting agent and one containing the substrate, and a cap for closing the vessel.

In another aspect, a device for testing if breast milk has a metal comprises a vessel for holding the sample of breast milk which already contains the substrate and two crushable ampoules, one containing the detecting agent and one containing the enzyme, and a cap for closing the vessel.

In an additional aspect, a device for testing if breast milk has a metal comprises a vessel for holding the sample of breast milk which contains three crushable ampoules one containing the detecting agent, one containing the substrate, and one containing the enzyme, and a cap for closing the vessel.

In another aspect, a device for testing if breast milk has a metal comprises a vessel for holding the sample of breast milk which already contains the enzyme and one crushable ampoule containing the detecting agent, and a cap for closing the vessel which already contains the substrate, which upon mixing enters the vessel.

In an additional aspect, a device for testing if breast milk has a metal comprises a vessel for holding the sample of breast milk which already contains the enzyme and one crushable ampoule containing the detecting agent, and a cap for closing the vessel which contains one crushable ampoule containing the substrate, which upon breaking enters the vessel.

In another aspect, a device for testing if breast milk has a metal comprises a vessel for holding the sample of breast milk containing two crushable ampoules, one containing the detecting agent and one containing the enzyme, and a cap for closing the vessel which already contains the substrate, which upon mixing enters the vessel.

In an additional aspect, a device for testing if breast milk has a metal comprises a vessel for holding the sample of breast milk containing two crushable ampoules, one containing the detecting agent and one containing the enzyme, and a cap for closing the vessel which contains one crushable ampoule containing the substrate, which upon mixing enters the vessel.

In another aspect, a device for testing if breast milk has a metal comprises a vessel for holding the sample of breast milk which already contains the substrate and one crushable ampoule containing the detecting agent, and a cap for closing the vessel which already contains the enzyme, which upon mixing enters the vessel.

In an additional aspect, a device for testing if breast milk has a metal comprises a vessel for holding the sample of breast milk which already contains the substrate and one crushable ampoule containing the detecting agent, and a cap for closing the vessel which contains one crushable ampoule containing the enzyme, which upon breaking enters the vessel.

In another aspect, a device for testing if breast milk has a metal comprises a vessel for holding the sample of breast milk which contains two crushable ampoules, one containing the detecting agent and one containing the substrate, and a cap for closing the vessel which already contains the enzyme, which upon mixing enters the vessel.

In an additional aspect, a device for testing if breast milk has a metal comprises a vessel for holding the sample of breast milk which contains two crushable ampoules, one containing the detecting agent and one containing the substrate, and a cap for closing the vessel which contains one crushable ampoule containing the enzyme, which upon breaking enters the vessel.

In another aspect, a device for testing if breast milk has a metal comprises a vessel for holding the sample of breast milk which contains one crushable ampoule containing the detecting agent, and a cap for closing the vessel which already contains both the enzyme and the substrate, which upon mixing enters the vessel.

In an additional aspect, a device for testing if breast milk has a metal comprises a vessel for holding the sample of breast milk which contains one crushable ampoule containing the detecting agent, and a cap for closing the vessel which already contains the enzyme and one crushable ampoule containing the substrate, which upon breaking and mixing enter the vessel.

In another aspect, a device for testing if breast milk has a metal comprises a vessel for holding the sample of breast milk which contains one crushable ampoule containing the detecting agent, and a cap for closing the vessel which already contains the substrate and one crushable ampoule containing the enzyme, which upon breaking and mixing enter the vessel.

In an additional aspect, a device for testing if breast milk has a metal comprises a vessel for holding the sample of breast milk which contains one crushable ampoule containing the detecting agent, and a cap for closing the vessel which contains two crushable ampoules, one containing the enzyme and one containing the substrate, which upon breaking enter the vessel.

In another aspect, a device for testing if breast milk has a metal comprises a vessel for holding the sample of breast milk which already contains both the enzyme and substrate and a cap for closing the vessel which already contains the detecting agent, which upon mixing enters the vessel.

In another aspect, a device for testing if breast milk has a metal comprises a vessel for holding the sample of breast milk which already contains both the enzyme and substrate, and a cap for closing the vessel which contains one crushable ampoule containing the detecting agent, which upon breaking enters the vessel.

In an additional aspect, a device for testing if breast milk has a metal comprises a vessel for holding the sample of breast milk which already contains the enzyme and one crushable ampoule containing the substrate, and a cap for closing the vessel which already contains the detecting agent, which upon mixing enters the vessel.

In another aspect, a device for testing if breast milk has a metal comprises a vessel for holding the sample of breast milk which already contains the enzyme and one crushable ampoule containing the substrate, and a cap for closing the vessel which contains one crushable ampoule containing the detecting agent, which upon breaking enters the vessel.

In an additional aspect, a device for testing if breast milk has a metal comprises a vessel for holding the sample of breast milk which already contains the enzyme, and a cap for closing the vessel which already contains both the detecting agent and the substrate, which upon mixing enter the vessel.

In another aspect, a device for testing if breast milk has a metal comprises a vessel for holding the sample of breast milk which already contains the enzyme, and a cap for closing the vessel which already contains the detecting agent and one crushable ampoule containing the substrate, which upon breaking and mixing enter the vessel.

In an additional aspect, a device for testing if breast milk has a metal comprises a vessel for holding the sample of breast milk which already contains the enzyme, and a cap for closing the vessel which already contains the substrate and one crushable ampoule containing the detecting agent, which upon breaking and mixing enter the vessel.

In another aspect, a device for testing if breast milk has a metal comprises a vessel for holding the sample of breast milk which already contains the enzyme, and a cap for closing the vessel which contains two crushable ampoules, one containing the detecting agent and one containing the substrate, which upon breaking and mixing enter the vessel.

In an additional aspect, a device for testing if breast milk has a metal comprises a vessel for holding the sample of breast milk which already contains the substrate and one crushable ampoule containing the enzyme, and a cap for closing the vessel which already contains the detecting agent, which upon mixing enters the vessel.

In another aspect, a device for testing if breast milk has a metal comprises a vessel for holding the sample of breast milk which already contains the substrate and one crushable ampoule containing the enzyme, and a cap for closing the vessel which contains one crushable ampoule containing the detecting agent, which upon breaking enters the vessel.

In another aspect, a device for testing if breast milk has a metal comprises a vessel for holding the sample of breast milk which contains two crushable ampoules, one containing the enzyme and one containing the substrate, and a cap for closing the vessel which already contains the detecting agent, which upon mixing enters the vessel is provided.

In an additional aspect, a device for testing if breast milk has a metal comprises a vessel for holding the sample of breast milk which contains two crushable ampoules, one containing the enzyme and one containing the substrate, and a cap for closing the vessel which contains one crushable ampoule containing the detecting agent, which upon breaking enters the vessel is described.

In another aspect, a device for testing if breast milk has a metal comprises a vessel for holding the sample of breast milk which contains one crushable ampoule containing the enzyme, and a cap for closing the vessel which already contains both the detecting agent and the substrate, which upon mixing enter the vessel is provided.

In an additional aspect, a device for testing if breast milk has a metal comprises a vessel for holding the sample of breast milk which contains one crushable ampoule containing the enzyme, and a cap for closing the vessel which already contains the detecting agent and one crushable ampoule containing the substrate, which upon breaking and mixing enter the vessel is described.

In another aspect, a device for testing if breast milk has a metal comprises a vessel for holding the sample of breast milk which contains one crushable ampoule containing the enzyme, and a cap for closing the vessel which already contains the substrate and one crushable ampoule containing the detecting agent, which upon breaking and mixing enter the vessel is provided.

In an additional aspect, a device for testing if breast milk has a metal comprises a vessel for holding the sample of breast milk which contains one crushable ampoule containing the enzyme, and a cap for closing the vessel which contains two crushable ampoules, one containing the detecting agent and one containing the substrate, which upon breaking enter the vessel is described.

In one aspect, a device for testing if breast milk has a metal comprises a vessel for holding the sample of breast milk which already contains the substrate, and a cap for closing the vessel which already contains the detecting agent and the enzyme, which upon mixing enter the vessel is provided.

In an additional aspect, a device for testing if breast milk has a metal comprises a vessel for holding the sample of breast milk which already contains the substrate, and a cap for closing the vessel which already contains the detecting agent and one crushable ampoule containing the enzyme, which upon breaking and mixing enter the vessel is described.

In another aspect, a device for testing if breast milk has a metal comprises a vessel for holding the sample of breast milk which already contains the substrate, and a cap for closing the vessel which already contains the enzyme and one crushable ampoule containing the detecting agent, which upon breaking and mixing enter the vessel is provided.

In an additional aspect, a device for testing if breast milk has a metal comprises a vessel for holding the sample of breast milk which already contains the substrate, and a cap for closing the vessel which contains two crushable ampoules, one containing the detecting agent and one containing the enzyme, which upon breaking enter the vessel is described.

In another aspect, a device for testing if breast milk has a metal comprises a vessel for holding the sample of breast milk which contains a crushable ampoule containing the substrate, and a cap for closing the vessel which already contains the detecting agent and the enzyme, which upon mixing enter the vessel is provided.

In an additional aspect, a device for testing if breast milk has a metal comprises a vessel for holding the sample of breast milk which contains a crushable ampoule containing the substrate, and a cap for closing the vessel which already contains the detecting agent and one crushable ampoule containing the enzyme, which upon breaking and mixing enter the vessel is described.

In another aspect, a device for testing if breast milk has a metal comprises a vessel for holding the sample of breast milk which contains a crushable ampoule containing the substrate, and a cap for closing the vessel which already contains the enzyme and one crushable ampoule containing the detecting agent, which upon breaking and mixing enter the vessel is disclosed.

In an additional aspect, a device for testing if breast milk has a metal comprises a vessel for holding the sample of breast milk which contains a crushable ampoule containing the substrate, and a cap for closing the vessel which contains two crushable ampoules, one containing the detecting agent and one containing the enzyme, which upon breaking enter the vessel.

In another aspect, a device for testing if breast milk has a metal comprises a vessel for holding the sample of breast milk, and a cap for closing the vessel which already contains the detecting agent, the enzyme, and the substrate, which upon mixing enter the vessel.

In an additional aspect, a device for testing if breast milk has a metal comprises a vessel for holding the sample of breast milk, and a cap for closing the vessel which already contains the detecting agent and the enzyme and one crushable ampoule containing the substrate, which upon breaking and mixing enter the vessel.

In another aspect, a device for testing if breast milk has a metal comprises a vessel for holding the sample of breast milk, and a cap for closing the vessel which already contains the detecting agent and the substrate and one crushable ampoule containing the enzyme, which upon breaking and mixing enter the vessel.

In an additional aspect, a device for testing if breast milk has a metal comprises a vessel for holding the sample of breast milk, and a cap for closing the vessel which already contains the detecting agent and two crushable ampoules one containing the enzyme and one containing the substrate, which upon breaking and mixing enter the vessel.

In another aspect, a device for testing if breast milk has a metal comprises a vessel for holding the sample of breast milk, and a cap for closing the vessel which already contains the enzyme and the substrate and one crushable ampoule containing the detecting agent, which upon breaking and mixing enter the vessel.

In an additional aspect, a device for testing if breast milk has a metal comprises a vessel for holding the sample of breast milk, and a cap for closing the vessel which already contains the enzyme and two crushable ampoules one containing the detecting agent and one containing the substrate, which upon breaking and mixing enter the vessel.

In another aspect, a device for testing if breast milk has a metal comprises a vessel for holding the sample of breast milk, and a cap for closing the vessel which already contains the substrate and two crushable ampoules one containing the detecting agent and one containing the enzyme, which upon breaking and mixing enter the vessel.

In an additional aspect, a device for testing if breast milk has a metal comprises a vessel for holding the sample of breast milk, and a cap for closing the vessel which contains three crushable ampoules one containing the detecting agent, one containing the enzyme, and one containing the substrate, which upon breaking enter the vessel.

In another aspect, a device for testing if breast milk has a metal comprises a vessel for holding the sample of breast milk which contains one crushable ampoule containing the detecting agent, the enzyme, and the substrate, and a cap for closing the vessel.

In an additional aspect, a device for testing if breast milk has a metal comprises a vessel for holding the sample of breast milk, and a cap for closing the vessel which contains one crushable ampoule containing the detecting agent, the enzyme, and the substrate, which upon breaking enter the vessel.

In another aspect, a device for testing if breast milk has a metal comprises a vessel for holding the sample of breast milk which already contains the substrate and one crushable ampoule containing both the detecting agent and the enzyme, and a cap for closing the vessel.

In an additional aspect, a device for testing if breast milk has a metal comprises a vessel for holding the sample of breast milk which contains two crushable ampoules, one containing both the detecting agent and the enzyme and one containing the substrate, and a cap for closing the vessel.

In another aspect, a device for testing if breast milk has a metal comprises a vessel for holding the sample of breast milk which contains one crushable ampoule containing both the detecting agent and the enzyme, and a cap for closing the vessel which already contains the substrate, which upon mixing enters the vessel.

In an additional aspect, a device for testing if breast milk has a metal comprises a vessel for holding the sample of breast milk which contains one crushable ampoule containing both the detecting agent and the enzyme, and a cap for closing the vessel which contains one crushable ampoule containing the substrate, which upon breaking enters the vessel.

In another aspect, a device for testing if breast milk has a metal comprises a vessel for holding the sample of breast milk which already contains the enzyme and one crushable ampoule containing both the detecting agent and the substrate, and a cap for closing the vessel.

In an additional aspect, a device for testing if breast milk has a metal comprises a vessel for holding the sample of breast milk which contains two crushable ampoules, one containing both the detecting agent and the substrate and one containing the enzyme, and a cap for closing the vessel.

In another aspect, a device for testing if breast milk has a metal comprises a vessel for holding the sample of breast milk which contains one crushable ampoule containing both the detecting agent and the substrate, and a cap for closing the vessel which already contains the enzyme, which upon mixing enters the vessel.

In an additional aspect, a device for testing if breast milk has a metal comprises a vessel for holding the sample of breast milk which contains one crushable ampoule containing both the detecting agent and the substrate, and a cap for closing the vessel which contains one crushable ampoule containing the enzyme, which upon breaking enters the vessel.

In another aspect, a device for testing if breast milk has a metal comprises a vessel for holding the sample of breast milk which already contains the detecting agent and one crushable ampoule containing both the enzyme and the substrate, and a cap for closing the vessel.

In an additional aspect, a device for testing if breast milk has a metal comprises a vessel for holding the sample of breast milk which contains two crushable ampoules, one containing both the enzyme and the substrate and one containing the detecting agent, and a cap for closing the vessel.

In another aspect, a device for testing if breast milk has a metal comprises a vessel for holding the sample of breast milk which contains one crushable ampoule containing both the enzyme and the substrate, and a cap for closing the vessel which already contains the detecting agent, which upon mixing enters the vessel.

In an additional aspect, a device for testing if breast milk has a metal comprises a vessel for holding the sample of breast milk which contains one crushable ampoule containing both the enzyme and the substrate, and a cap for closing the vessel which contains one crushable ampoule containing the detecting agent, which upon breaking enters the vessel.

In another aspect, a device for testing if breast milk has a metal comprises a vessel for holding the sample of breast milk which already contains the substrate, and a cap for closing the vessel which contains one crushable ampoule containing both the detecting agent and the enzyme, which upon breaking enters the vessel.

In an additional aspect, a device for testing if breast milk has a metal comprises a vessel for holding the sample of breast milk which contains one crushable ampoule containing the substrate, and a cap for closing the vessel which contains one crushable ampoule containing both the detecting agent and the enzyme, which upon breaking enters the vessel.

In another aspect, a device for testing if breast milk has a metal comprises a vessel for holding the sample of breast milk, and a cap for closing the vessel which already contains the substrate and one crushable ampoule containing both the detecting agent and enzyme, which upon breaking and mixing enter the vessel.

In an additional aspect, a device for testing if breast milk has a metal comprises a vessel for holding the sample of breast milk, and a cap for closing the vessel which contains two crushable ampoules, one containing the substrate and one containing both the detecting agent and the enzyme, which upon breaking enter the vessel.

In another aspect, a device for testing if breast milk has a metal comprises a vessel for holding the sample of breast milk which already contains the enzyme, and a cap for closing the vessel which contains one crushable ampoule containing both the detecting agent and the substrate, which upon breaking enters the vessel.

In an additional aspect, a device for testing if breast milk has a metal comprises a vessel for holding the sample of breast milk which contains one crushable ampoule containing the enzyme, and a cap for closing the vessel which contains one crushable ampoule containing both the detecting agent and the substrate, which upon breaking enters the vessel.

In another aspect, a device for testing if breast milk has a metal comprises a vessel for holding the sample of breast milk, and a cap for closing the vessel which already contains the enzyme and one crushable ampoule containing both the detecting agent and substrate, which upon breaking and mixing enter the vessel.

In an additional aspect, a device for testing if breast milk has a metal comprises a vessel for holding the sample of breast milk, and a cap for closing the vessel which contains two crushable ampoules, one containing the enzyme and one containing both the detecting agent and the substrate, which upon breaking enter the vessel.

In another aspect, a device for testing if breast milk has a metal comprises a vessel for holding the sample of breast milk which already contains the detecting agent, and a cap for closing the vessel which contains one crushable ampoule containing both the enzyme and the substrate, which upon breaking enters the vessel.

In an additional aspect, a device for testing if breast milk has a metal comprises a vessel for holding the sample of breast milk which contains one crushable ampoule containing the detecting agent, and a cap for closing the vessel which contains one crushable ampoule containing both the enzyme and the substrate, which upon breaking enters the vessel.

In another aspect, a device for testing if breast milk has a metal comprises a vessel for holding the sample of breast milk, and a cap for closing the vessel which already contains the detecting agent and one crushable ampoule containing both the enzyme and substrate, which upon breaking and mixing enter the vessel.

In an additional aspect, a device for testing if breast milk has a metal comprises a vessel for holding the sample of breast milk, and a cap for closing the vessel which contains two crushable ampoules, one containing the detecting agent and one containing both the enzyme and the substrate, which upon breaking enter the vessel.

In another aspect, a device for testing if breast milk has a metal comprises a vessel for holding the sample of breast milk which already contains the detecting agent and the enzyme, and a cap for closing the vessel.

In an additional aspect, a device for testing if breast milk has a metal comprises a vessel for holding the sample of breast milk which already contains the detecting agent and one crushable ampoule containing the enzyme, and a cap for closing the vessel.

In another aspect, a device for testing if breast milk has a metal comprises a vessel for holding the sample of breast milk which already contains the detecting agent, and a cap for closing the vessel which already contains the enzyme, which upon mixing enters the vessel.

In an additional aspect, a device for testing if breast milk has a metal comprises a vessel for holding the sample of breast milk which already contains the detecting agent, and a cap for closing the vessel which contains one crushable ampoule containing the enzyme, which upon breaking enters the vessel.

In another aspect, a device for testing if breast milk has a metal comprises a vessel for holding the sample of breast milk which already contains the enzyme and one crushable ampoule containing the detecting agent, and a cap for closing the vessel.

In an additional aspect, a device for testing if breast milk has a metal comprises a vessel for holding the sample of breast milk which contains two crushable ampoules, one containing the detecting agent and one containing the enzyme, and a cap for closing the vessel.

In another aspect, a device for testing if breast milk has a metal comprises a vessel for holding the sample of breast milk which contains one crushable ampoule containing the detecting agent, and a cap for closing the vessel which already contains the enzyme, which upon mixing enters the vessel.

In an additional aspect, a device for testing if breast milk has a metal comprises a vessel for holding the sample of breast milk which contains one crushable ampoule containing the detecting agent, and a cap for closing the vessel which contains one crushable ampoule containing the enzyme, which upon breaking enters the vessel.

In another aspect, a device for testing if breast milk has a metal comprises a vessel for holding the sample of breast milk which already contains the enzyme, and a cap for closing the vessel which already contains the detecting agent, which upon mixing enters the vessel.

In an additional aspect, a device for testing if breast milk has a metal comprises a vessel for holding the sample of breast milk which already contains the enzyme, and a cap for closing the vessel which contains one crushable ampoule containing the detecting agent, which upon breaking enters the vessel.

In another aspect, a device for testing if breast milk has a metal comprises a vessel for holding the sample of breast milk which contains one crushable ampoule containing the enzyme, and a cap for closing the vessel which already contains the detecting agent, which upon mixing enters the vessel.

In an additional aspect, a device for testing if breast milk has a metal comprises a vessel for holding the sample of breast milk which contains one crushable ampoule containing the enzyme, and a cap for closing the vessel which contains one crushable ampoule containing the detecting agent, which upon breaking enters the vessel.

In another aspect, a device for testing if breast milk has a metal comprises a vessel for holding the sample of breast milk, and a cap for closing the vessel which already contains both the detecting agent and the enzyme, which upon mixing enters the vessel.

In an additional aspect, a device for testing if breast milk has a metal comprises a vessel for holding the sample of breast milk, and a cap for closing the vessel which already contains the detecting agent and one crushable ampoule containing the enzyme, which upon breaking and mixing enters the vessel.

In another aspect, a device for testing if breast milk has a metal comprises a vessel for holding the sample of breast milk, and a cap for closing the vessel which already contains the enzyme and one crushable ampoule containing the detecting agent, which upon breaking and mixing enters the vessel.

In an additional aspect, a device for testing if breast milk has a metal comprises a vessel for holding the sample of breast milk, and a cap for closing the vessel which contains two crushable ampoules, one containing the detecting agent and one containing the enzyme, which upon breaking enter the vessel.

In another aspect, a device for testing if breast milk has a metal comprises a vessel for holding the sample of breast milk which contains one crushable ampoule containing both the detecting agent and the enzyme, and a cap for closing the vessel.

In an additional aspect, a device for testing if breast milk has a metal comprises a vessel for holding the sample of breast milk, and a cap for closing the vessel which contains one crushable ampoule containing both the detecting agent and the enzyme, which upon breaking enter the vessel.

In another aspect, a device for testing if breast milk has a metal comprises a vessel for holding the sample of breast milk which already contains the enzyme and the substrate, and a cap for closing the vessel.

In an additional aspect, a device for testing if breast milk has a metal comprises a vessel for holding the sample of breast milk which already contains the substrate and one crushable ampoule containing the enzyme, and a cap for closing the vessel.

In another aspect, a device for testing if breast milk has a metal comprises a vessel for holding the sample of breast milk which already contains the substrate, and a cap for closing the vessel which already contains the enzyme, which upon mixing enters the vessel.

In an additional aspect, a device for testing if breast milk has a metal comprises a vessel for holding the sample of breast milk which already contains the substrate, and a cap for closing the vessel which contains one crushable ampoule containing the enzyme, which upon breaking enters the vessel.

In another aspect, a device for testing if breast milk has a metal comprises a vessel for holding the sample of breast milk which already contains the enzyme and one crushable ampoule containing the substrate, and a cap for closing the vessel.

In an additional aspect, a device for testing if breast milk has a metal comprises a vessel for holding the sample of breast milk which contains two crushable ampoules, one containing the enzyme and one containing the substrate, and a cap for closing the vessel.

In another aspect, a device for testing if breast milk has a metal comprises a vessel for holding the sample of breast milk which contains one crushable ampoule containing the substrate, and a cap for closing the vessel which already contains the enzyme, which upon mixing enters the vessel.

In an additional aspect, a device for testing if breast milk has a metal comprises a vessel for holding the sample of breast milk which contains one crushable ampoule containing the substrate, and a cap for closing the vessel which contains one crushable ampoule containing the enzyme, which upon breaking enters the vessel.

In another aspect, a device for testing if breast milk has a metal comprises a vessel for holding the sample of breast milk which already contains the enzyme, and a cap for closing the vessel which already contains the substrate, which upon mixing enters the vessel.

In an additional aspect, a device for testing if breast milk has a metal comprises a vessel for holding the sample of breast milk which already contains the enzyme, and a cap for closing the vessel which contains one crushable ampoule containing the substrate, which upon breaking enters the vessel.

In another aspect, a device for testing if breast milk has a metal comprises a vessel for holding the sample of breast milk which contains one crushable ampoule containing the enzyme, and a cap for closing the vessel which already contains the substrate, which upon mixing enters the vessel.

In an additional aspect, a device for testing if breast milk has a metal comprises a vessel for holding the sample of breast milk which contains one crushable ampoule containing the enzyme, and a cap for closing the vessel which contains one crushable ampoule containing the substrate, which upon breaking enters the vessel.

In another aspect, a device for testing if breast milk has a metal comprises a vessel for holding the sample of breast milk, and a cap for closing the vessel which already contains both the enzyme and the substrate, which upon mixing enters the vessel.

In an additional aspect, a device for testing if breast milk has a metal comprises a vessel for holding the sample of breast milk, and a cap for closing the vessel which already contains the substrate and one crushable ampoule containing the enzyme, which upon breaking and mixing enters the vessel.

In another aspect, a device for testing if breast milk has a metal comprises a vessel for holding the sample of breast milk, and a cap for closing the vessel which already contains the enzyme and one crushable ampoule containing the substrate, which upon breaking and mixing enters the vessel.

In an additional aspect, a device for testing if breast milk has a metal comprises a vessel for holding the sample of breast milk, and a cap for closing the vessel which contains two crushable ampoules, one containing the enzyme and one containing the substrate, which upon breaking enter the vessel.

In another aspect, a device for testing if breast milk has a metal comprises a vessel for holding the sample of breast milk which contains one crushable ampoule containing both the enzyme and the substrate, and a cap for closing the vessel.

In an additional aspect, a device for testing if breast milk has a metal comprises a vessel for holding the sample of breast milk, and a cap for closing the vessel which contains one crushable ampoule containing both the enzyme and the substrate, which upon breaking enter the vessel.

In another aspect, a device for testing if breast milk has a metal comprises a vessel for holding the sample of breast milk which already contains the detecting agent and the substrate, and a cap for closing the vessel.

In an additional aspect, a device for testing if breast milk has a metal comprises a vessel for holding the sample of breast milk which already contains the substrate and one crushable ampoule containing the detecting agent, and a cap for closing the vessel.

In another aspect, a device for testing if breast milk has a metal comprises a vessel for holding the sample of breast milk which already contains the substrate, and a cap for closing the vessel which already contains the detecting agent, which upon mixing enters the vessel.

In an additional aspect, a device for testing if breast milk has a metal comprises a cap for closing the vessel which already contains the detecting agent, which upon mixing enters the vessel., and a cap for closing the vessel which contains one crushable ampoule containing the detecting agent, which upon breaking enters the vessel.

In another aspect, a device for testing if breast milk has a metal comprises a vessel for holding the sample of breast milk which already contains the detecting agent and one crushable ampoule containing the substrate, and a cap for closing the vessel.

In an additional aspect, a device for testing if breast milk has a metal comprises a vessel for holding the sample of breast milk which contains two crushable ampoules, one containing the detecting agent and one containing the substrate, and a cap for closing the vessel.

In another aspect, a device for testing if breast milk has a metal comprises a vessel for holding the sample of breast milk which contains one crushable ampoule containing the substrate, and a cap for closing the vessel which already contains the detecting agent, which upon mixing enters the vessel.

In an additional aspect, a device for testing if breast milk has a metal comprises a vessel for holding the sample of breast milk which contains one crushable ampoule containing the substrate, and a cap for closing the vessel which contains one crushable ampoule containing the detecting agent, which upon breaking enters the vessel.

In another aspect, a device for testing if breast milk has a metal comprises a vessel for holding the sample of breast milk which already contains the detecting agent, and a cap for closing the vessel which already contains the substrate, which upon mixing enters the vessel.

In an additional aspect, a device for testing if breast milk has a metal comprises a vessel for holding the sample of breast milk which already contains the detecting agent, and a cap for closing the vessel which contains one crushable ampoule containing the substrate, which upon breaking enters the vessel.

In another aspect, a device for testing if breast milk has a metal comprises a vessel for holding the sample of breast milk which contains one crushable ampoule containing the detecting agent, and a cap for closing the vessel which already contains the substrate, which upon mixing enters the vessel.

In an additional aspect, a device for testing if breast milk has a metal comprises a vessel for holding the sample of breast milk which contains one crushable ampoule containing the detecting agent, and a cap for closing the vessel which contains one crushable ampoule containing the substrate, which upon breaking enters the vessel.

In another aspect, a device for testing if breast milk has a metal comprises a vessel for holding the sample of breast milk, and a cap for closing the vessel which already contains both the detecting agent and the substrate, which upon mixing enters the vessel.

In an additional aspect, a device for testing if breast milk has a metal comprises a vessel for holding the sample of breast milk, and a cap for closing the vessel which already contains the substrate and one crushable ampoule containing the detecting agent, which upon breaking and mixing enters the vessel.

In another aspect, a device for testing if breast milk has a metal comprises a vessel for holding the sample of breast milk, and a cap for closing the vessel which already contains the detecting agent and one crushable ampoule containing the substrate, which upon breaking and mixing enters the vessel.

In an additional aspect, a device for testing if breast milk has a metal comprises a vessel for holding the sample of breast milk, and a cap for closing the vessel which contains two crushable ampoules, one containing the detecting agent and one containing the substrate, which upon breaking enter the vessel.

In another aspect, a device for testing if breast milk has a metal comprises a vessel for holding the sample of breast milk which contains one crushable ampoule containing both the detecting agent and the substrate, and a cap for closing the vessel.

In an additional aspect, a device for testing if breast milk has a metal comprises a vessel for holding the sample of breast milk, and a cap for closing the vessel which contains one crushable ampoule containing both the detecting agent and the substrate, which upon breaking enter the vessel.

In another aspect, a device for testing if breast milk has a metal comprises a vessel for holding the sample of breast milk which already contains the detecting agent, and a cap for closing the vessel.

In an additional aspect, a device for testing if breast milk has a metal comprises a vessel for holding the sample of breast milk which contains one crushable ampoule containing the detecting agent, and a cap for closing the vessel.

In another aspect, a device for testing if breast milk has a metal comprises a vessel for holding the sample of breast milk, and a cap for closing the vessel which already contains the detecting agent, which upon mixing enters the vessel.

In an additional aspect, a device for testing if breast milk has a metal comprises a vessel for holding the sample of breast milk, and a cap for closing the vessel which contains one breakable ampoule containing the detecting agent, which upon breaking enters the vessel.

In another aspect, a device for testing if breast milk has a metal comprises a vessel for holding the sample of breast milk which already contains the enzyme, and a cap for closing the vessel.

In an additional aspect, a device for testing if breast milk has a metal comprises a vessel for holding the sample of breast milk which contains one crushable ampoule containing the enzyme, and a cap for closing the vessel.

In another aspect, a device for testing if breast milk has a metal comprises a vessel for holding the sample of breast milk, and a cap for closing the vessel which already contains the enzyme, which upon mixing enters the vessel.

In an additional aspect, a device for testing if breast milk has a metal comprises a vessel for holding the sample of breast milk, and a cap for closing the vessel which contains one breakable ampoule containing the enzyme, which upon breaking enters the vessel.

In another aspect, a device for testing if breast milk has a metal comprises a vessel for holding the sample of breast milk which already contains the substrate, and a cap for closing the vessel.

In an additional aspect, a device for testing if breast milk has a metal comprises a vessel for holding the sample of breast milk which contains one crushable ampoule containing the substrate, and cap for closing the vessel.

In another aspect, a device for testing if breast milk has a metal comprises a vessel for holding the sample of breast milk, and a cap for closing the vessel which already contains the substrate, which upon mixing enters the vessel.

In an additional aspect, a device for testing if breast milk has a metal comprises a vessel for holding the sample of breast milk, and a cap for closing the vessel which contains one breakable ampoule containing the substrate, which upon breaking enters the vessel.

In certain examples the crushable ampoule is composed of glass, polymer, metal, ceramic or combinations thereof.

In other examples the vessel is a vial, cup, mug, chamber, container, beaker, syringe, goblet, reservoir composed of glass, polymer, metal, ceramic or combinations thereof.

In further examples the vessel is marked with a graduated scale so as to add a specific, known, volume of milk.

In certain embodiments the cap is composed of glass, polymer, metal, ceramic or combinations thereof.

In some embodiments the cap is a screw cap, twist, zip-tie, pinch, stopper, or snap cap.

In certain examples the sample of breast milk is a sample of mammalian breast milk. In further examples the sample of mammalian breast milk is primate, bovine, ovine, caprine, equine, porcine, murine, feline, or canine. In one example the sample is human.

In additional embodiments the device further comprises a medicament, colorant, flavoring, scent, fibrous additive, antioxidant, thickener, or plasticizer.

In some examples a method comprising the step of determining if a sample of breast milk has a metal using the device is disclosed.

In certain examples a method comprising the steps of determining if a sample of breast milk has a metal using the device whereby 1000 to 500 mL of breast milk are used is disclosed. In other examples a method comprising the steps of determining if a sample of breast milk has a metal using the device whereby 500 to 100 mL of breast milk are used is provided. In another example a method comprising the steps of determining if a sample of breast milk has a metal using the device whereby 100-50 mL of breast milk are used is described. In an additional example a method comprising the steps of determining if a sample of breast milk has a metal using the device whereby 50-10 mL of breast milk are used is disclosed. In certain embodiments a method comprising the steps of determining if a sample of breast milk has a metal using the device whereby 10-1 mL of breast milk are used is provided. In some embodiments a method comprising the steps of determining if a sample of breast milk has a metal using the device whereby 1-0.1 mL of breast milk are used is described. In other embodiments a method comprising the steps of determining if a sample of breast milk has a metal using the device whereby 0.1-0.01 mL of breast milk are used is disclosed. In another embodiment a method comprising the steps of determining if a sample of breast milk has a metal using the device whereby 0.01-0.001 mL of breast milk are used is described. In an additional embodiment a method comprising the steps of determining if a sample of breast milk has a metal using the device whereby 0.001-0.0001 mL of breast milk are used is provided.

In a certain example a method of testing comprising the steps of first adding a detecting agent to a milk sample to give a mixture, and second adding an enzyme and third adding a substrate to the mixture is disclosed. In another example a method of testing comprising the steps of first adding a detecting agent to a milk sample to give a mixture, and second adding a substrate and third adding an enzyme to the mixture is described. In other examples a method of testing comprising the steps of first adding an enzyme to a milk sample to give a mixture, and second adding a detecting agent and third adding a substrate to the mixture is provided.

In some examples a method of testing comprising the steps of first adding an enzyme to a milk sample to give a mixture, and second adding a substrate and third adding a detecting agent to the mixture is disclosed. In another example a method of testing comprising the steps of first adding a substrate to a milk sample to give a mixture, and second adding a detecting agent and third adding an enzyme to the mixture is described. In a further example a method of testing comprising the steps of first adding a substrate to a milk sample to give a mixture, and second adding an enzyme and third adding a detecting agent to the mixture is disclosed. In other examples a method of testing comprising the steps of first adding a detecting agent to a milk sample to give a mixture, and second adding an enzyme and substrate together to the mixture is provided. In some examples a method of testing comprising the steps of first adding an enzyme and a substrate together to a milk sample to give a mixture, and second adding a detecting agent to the mixture is disclosed. In another example a method of testing comprising the steps of first adding an enzyme to a milk sample to give a mixture, and second adding a detecting agent and substrate together to the mixture is described. In an additional example a method of testing comprising the steps of first adding a detecting agent and a substrate together to a milk sample to give a mixture, and second adding an enzyme to the mixture is disclosed. In certain embodiments a method of testing comprising the steps of first adding a substrate to a milk sample to give a mixture, and second adding a detecting agent and enzyme together to the mixture is disclosed. In some embodiments a method of testing comprising the steps of first adding a detecting agent and an enzyme together to a milk sample to give a mixture, and second adding a substrate to the mixture is described.

In some embodiments the method of testing comprising the step of adding a detecting agent, an enzyme, and a substrate together to a milk sample to provide a mixture is disclosed. In other embodiments the method of testing comprising the steps of first adding a detecting agent to a milk sample, and second adding an enzyme to provide a mixture is provided. In certain embodiments the method of testing comprising the steps of first adding an enzyme to a milk sample, and second adding a detecting agent to provide a mixture is described. In additional embodiments the method of testing comprising the step of first adding a detecting agent and an enzyme together to a milk sample is described. In another embodiment the method of testing comprising the steps of first adding a detecting agent to a milk sample, and second adding a substrate to provide a mixture is disclosed. In some embodiments the method of testing comprising the steps of first adding a substrate to a milk sample, and second adding a detecting agent to provide a mixture is provided. In another embodiment the method of testing comprising the step of first adding a detecting agent and a substrate together to provide a milk sample is described. In some examples the method of testing comprising the steps of first adding an enzyme to a milk sample, and second adding a substrate to provide a mixture is disclosed. In further examples the method of testing comprising the steps of first adding a substrate to a milk sample, and second adding an enzyme to provide a mixture is provided. In an additional example the method of testing comprising the step of first adding an enzyme and a substrate together to a milk sample is described. In another embodiment the method of testing comprising the step of adding a detecting agent to a milk sample is provided. In certain embodiments the method of testing comprising the step of adding an enzyme to a milk sample is described. In some embodiments the method of testing comprising the step of adding a substrate to a milk sample is disclosed.

In other embodiments sterilization of the device is conducted utilizing visible light irradiation, ultraviolet light, electron-beam radiation, gamma-radiation, chemical techniques, physical techniques, or combinations thereof. In some examples the sterilization of said device utilizes chemical techniques; and said chemical techniques comprise exposure to ethylene oxide or hydrogen peroxide vapor. In other examples the sterilization of the device utilizes physical techniques; and the physical techniques comprise moist heating, dry heating, retort and hot-fill canning, or filtration. In certain examples the sterilization of the device utilizes electron-beam radiation or gamma-radiation; and the amount of said radiation is between about 2 and about 40 kGy. In an additional example the sterilization of the device utilizes electron-beam radiation or gamma-radiation; and the amount of said radiation is between about 3 and about 20 kGy. In another example the sterilization of said device utilizes electron-beam radiation or gamma-radiation; and the amount of said radiation is between about 5 and about 12 kGy. In other embodiments the radiation is applied once or more than once. In some embodiments the amount of the radiation is between about 5 and about 40 kGy.

In certain embodiments sterilization of the device is conducted below about 150° C. In additional embodiments sterilization of the device is conducted below about 100° C. In another embodiment sterilization of the device is conducted below about 50° C. In further embodiments sterilization of the device is conducted below about 30° C. In other examples sterilization of the device is conducted below about 20° C. In certain examples sterilization of the device is conducted below about 10° C. In another example sterilization of the device is conducted below about 0° C.

In an additional example the sample of breast milk is from a primate, bovine, ovine, caprine, equine, porcine, murine, feline, or canine. In another example the sample of breast milk is from a human.

In certain embodiments the method comprising the steps of monitoring the breast milk for metals over a period of time is described. In some embodiments the monitoring period of time is about six months to about one year. In other embodiments the monitoring period of time is about six months. In further embodiments the monitoring period of time is about one year.

In another embodiment a kit is described which comprises instructions for use thereof. In an additional embodiment a kit is disclosed which comprises one or more devices and an instruction manual. In certain embodiments a kit is described which comprises one or more devices, a delivery system, and an instruction manual. In some examples a kit is disclosed which comprises one or more devices, a delivery system, an instruction manual and a logbook for recording the history of readings. In further examples a kit is described which comprises one or more devices, a delivery system, an instruction manual and a chart for plotting the history of readings. In some examples a kit is described which comprises one or more devices, a delivery system, an instruction manual, and an instruction booklet on how to record the history of readings on a secured on-line website.

In another example the delivery system is a syringe, a spoon, a pipette, an eye dropper, teaspoon, tablespoon, or a capillary tube.

In some examples the kit further comprises a desiccant or an antioxidant. In certain the antioxidant is selected from the group consisting of sodium metabisulfite, citric acid, and ascorbic acid.

In other examples the kit further comprises the device in an inert atmosphere.

In some embodiments the kit has a sterility assurance level of at least about 10⁻³. In other embodiments the kit has a sterility assurance level of at least about 10⁻⁶.

In further embodiments the kit includes a moisture-barrier element with a moisture vapor transmission rate (MVTR) less than or equal to about 0.15 gram per 100 square inches per day. In an additional embodiment the kit includes a moisture-barrier element with a moisture vapor transmission rate (MVTR) less than or equal to about 0.02 gram per 100 square inches per day. In some embodiments the moisture-barrier element comprises the device. In certain embodiments the moisture-barrier element comprises the cartridge. In other embodiments the moisture-barrier element comprises the counter.

In further examples the kit is protected from light.

In an additional example the kit is disposable.

In another example the kit is recyclable.

In certain examples the kit can be used in the home, workplace, clinic, outpatient office, milk bank, hospital, train, airplane, boat, car, and outdoors.

In another example a concentration-type assay test device for determining if a sample of breast milk has a metal comprising a detecting agent, enzyme, and substrate where an incomplete enzyme-substrate reaction occurs between the enzyme and the substrate in breast milk such that the detecting agent changes is disclosed.

In some examples the method whereby an incomplete enzyme-substrate reaction occurs between the enzyme and the substrate in breast milk such that the detecting agent changes is provided.

An examination of the following drawings will provide clarity in regards to the unique aspects of this versatile design.

### BRIEF DESCRIPTION OF THE FIGURES

FIG. 1 is a front sectional view of the adapter in use.

FIG. 2 is a front sectional view of the adapter comprised of a single unitary piece including a rubber nipple for drinking.

FIG. 3 is a front sectional view of another embodiment of the invention comprised of a unitary sipper-type adapter.

FIG. 4 is a front sectional view of another embodiment of the invention comprised of a unitary straw-type adapter with adhered drinking nipple.

FIG. 5 is a front sectional view of another embodiment of the invention comprised of an adapter with external threading that allows for a standard rubber nipple and nipple clamp ring to be secured.

FIG. 6 is a front sectional view of another embodiment of the invention comprised of an adapter that accepts a variety of interchangeable secondary pieces.

FIG. 7 Reaction scheme for the formation of the gel-clot by interaction between endotoxins and components of LAL.

FIG. 8 From left to right vials contained 0.5, 0.25, 0.125, 0.0625, 0.0312, and 0 EU/mL of endotoxin. Gel formation is only seen in the first three conditions.

FIG. 9 From left to right vials contained 0.5, 0.25, 0.125, 0.0625, 0.0312, and 0 EU/mL of endotoxin plus a control of just milk without LAL addition. Gel formation is only seen in the first three conditions.

FIG. 10 From left to right vials contained 0.5, 0.25, 0.125, 0.0625, 0.0312, and 0 EU/mL of endotoxin plus a control of just milk without LAL addition. Gel formation is seen in the first five conditions.

FIG. 11 (left) Internal view of the proposed device design. Two crushable ampoules are shown with false coloring to increase visualization. The paper sleeve to aid in the ampoule breakage is also shown. (right) External view of device after gelation and inversion.

FIG. 12 Contact angles of different fat milk content with PTFE, glass, and PDMS (n=3).

FIG. 13 Schematic prototype of the caloric monitor for proof-of-concept studies.

FIG. 14 Correlation curve of breast milk lipid content vs. time of passage through the detection cell in the prototype monitor (n=3).

FIG. 15 96-well microplate with varied levels of both mercury and urease. Bluer colors represent enzyme activity with green and yellow representing some inhibition due to mercury or due to lesser amounts of the enzyme. Color gradient is linear and by varying the amount of enzyme we can detect different amounts of mercury reliably.

FIG. 16 Overnight color development with the pH indicating dye system in infant formula. Tubes on the right represent decreasing levels of mercury with the red-boxed region on each tube blown up below for color comparison. The tubes on the right show the system with 500 ppb Hg but without urease and urea to show that color development is dependent on the combination of the two; (bottom) Plot of the Blue:Red ratio for the developed colors in the above images.

FIG. 17 (Left) Internal view of the proposed device design. Two crushable ampoules are shown: Purple containing the urease and Orange containing the dye and urea. (Right) External view of device after color development. User rotates the cardboard cover (white) until the viewing color in the viewing window matches the gradient color on the bottom.

FIG. 18 Sample outcome table depending on the weight of the child (horizontal dimension) and color reading recorded by the monitor (vertical dimension). The user would see a designation (+) which would encourage them to consult their physician, or (−) which would inform them their levels meet the US ASTDR recommendations. The table will be encased in a movable sleeve with a slit allowing viewing of a single column at once allowing the user to dial in the weight of the nursing infant.

FIG. 19 Monitor kit scheme. Top: disposable cartridge, Bottom: caloric counter.

FIG. 20 relates to Example 1 showing in a photograph the 4 pieces of the described mold open (left) and closed (right).

FIG. 21 shows a photographic close-up view of a completed unitary silicone adapter described in Example 2.

FIG. 22 is a photograph of the same adapter from Example 2 in use on a bottle.

FIG. 23 is another photograph of the same adapter from Example 2 in use on an inverted bottle.

FIG. 24 is a photograph of the dual elastomer adapter described in Example 3.

FIG. 25 Results of spoilage detection using the sodium hydroxide, phenolphthalein detection method. The colorless vial on the left represents high Dornic acidity while the pink vial on the right has a low Dornic.

FIG. 26 Spoilage detection using the tetrazolium method. The brown vial on the left contains formula with a low bacteria count/Dornic acidity, the brown vial in the center is breast milk with a low Dornic measurement, and the yellow vial on the right is breast milk with a high bacteria count/Dornic measurement.

FIG. 27 Internal view of the proposed spoilage device design. Two crushable ampoules are shown: Purple containing the dye and Orange containing the base.

FIG. 28 (Left) prototype spoilage testers with 7° D 4' dilution infant formula samples and (right) 9° D 4× dilution infant formula samples after crushing the ampoules and shaking.

FIG. 29 From left to right vials contained 0.5, 0.25, 0.125, 0.0625, 0.0312, and 0 EU/mL of endotoxin. Gel formation is only seen in the first three conditions.

FIG. 30 From left to right vials contained 0.5, 0.25, 0.125, 0.0625, 0.0312, and 0 EU/mL of endotoxin plus a control of just milk without LAL addition. Gel formation is only seen in the first three conditions.

FIG. 31 From left to right vials contained 0.5, 0.25, 0.125, 0.0625, 0.0312, and 0 EU/mL of endotoxin plus a control of just milk without LAL addition. Gel formation is seen in the first five conditions.

FIG. 32 Two crushable ampoules are shown with false coloring to increase visualization. The paper sleeve to aid in the ampoule breakage is also shown. (right) External view of device after gelation and inversion.

FIG. 33 Contact angles of different fat milk content with PTFE, glass, and PDMS.

FIG. 34 Schematic of the caloric monitor.

FIG. 35 Correlation curve of breast milk lipid content vs. time of passage through the detection cell in the prototype monitor.

FIG. 36 96-Well microplate with varied levels of both mercury and urease. Bluer colors represent enzyme activity with green and yellow representing some inhibition due to mercury or due to lesser amounts of the enzyme. Color gradient is linear and by varying the amount of enzyme we can detect different amounts of mercury reliably.

FIG. 37 Overnight color development with the pH indicating dye system in infant formula. Tubes on the right represent decreasing levels of mercury with the red-boxed region on each tube blown up below for color comparison. The tubes on the right show the system with 500 ppb Hg but without urease and urea to show that color development is dependent on the combination of the two.

FIG. 38 Plot of the Blue:Red ratio for the developed colors from FIG. 37.

FIG. 39 Breast milk without mercury (Right) and with mercury (Left) after 1.5 hours of development using the urease/urea/dye system.

FIG. 40 (top) Internal view of the proposed device design. Two crushable ampoules are shown with false coloring of the powder to increase visualization. (bottom) External view of device after hypothetical color development. User rotates the cardboard cover (white) until the color in the viewing window matches the gradient color on the bottom.

FIG. 41 Sample outcome table depending on the weight of the child (horizontal dimension) and color reading recorded by the monitor (vertical dimension). The user would see a designation (+) which would encourage them to consult their physician, or (−) which would inform them their levels meet the US ASTDR recommendations. The table will be encased in a movable sleeve with a slit allowing viewing of a single column at once allowing the user to dial in the weight of the nursing infant.

FIG. 42 A plot of Drop Count versus Fat Concentration (Creamatocrit %) having a correlation of r=0.9779.

### DETAILED DESCRIPTION

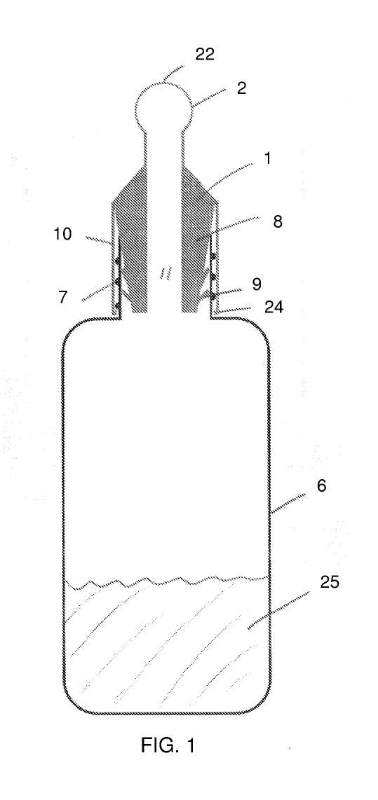

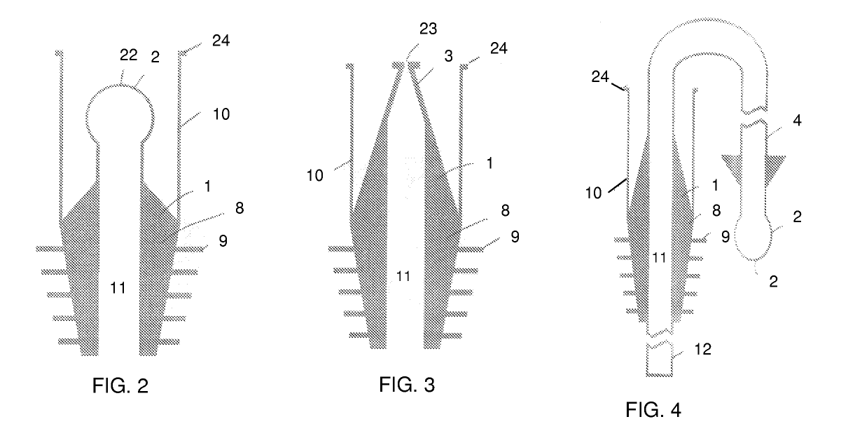

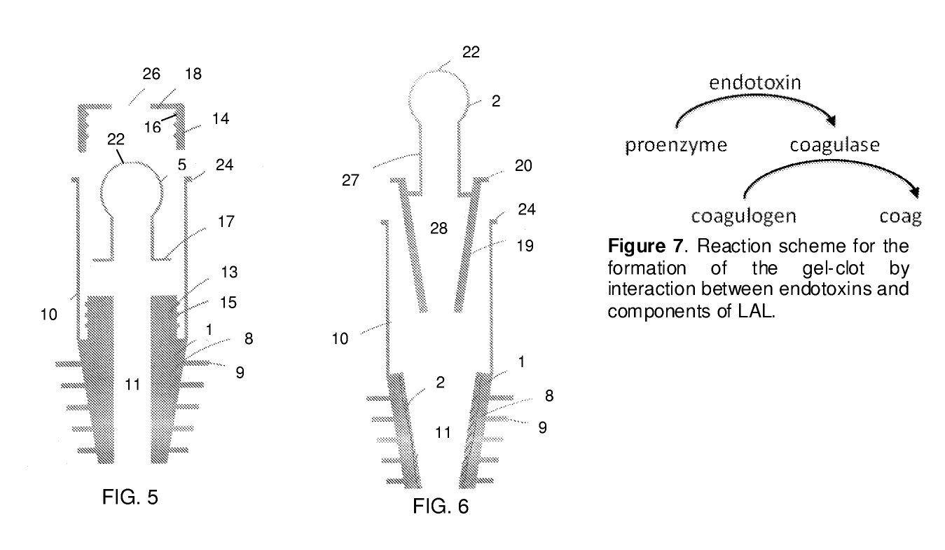

As shown in FIG. 1 of the present invention, an adapter 1 is inserted into the neck opening 7 of a glass, plastic, ceramic, or metal bottle 6 containing a fluid 25 such as water, milk, juice, soda, mineral water, infant formula, or sports drink. The adapter is made of a flexible material and consists of a tapered plug 8 that contains an internal channel to allow fluid flow 11. The periphery of the adapter body contains a series of flexible annular rings 9 that when inserted, friction seal against the interior of the bottle neck 7 to prevent liquid 25 leakage. The adapter also contains a long flexible flange 10 that is pulled over the exterior of the bottle neck 7 to add additional fixation to the adapter and further prevent any fluid loss. The flexible flange may contain a plurality of circumferential annular ribs 24 or other such structure that add additional strength and tear resistance to the material. A variety of unitary embodiments are envisioned where the adapter 1 is unitarily manufactured attached to a infant drinking nipple 2 containing a hole 22 that allows liquid to pass through, as shown in FIG. 2. Another embodiment shown in FIG. 3 is a sipper-type attachment 3 that allows removal of the fluid through an opening 23. Another embodiment of the invention shown in FIG. 4 is where a flexible tube is used to remove fluid from the bottle 6. This embodiment may contain an external tube 4 and/or internal tube 12. The external portion of the tube may be terminated in either a nipple 2, sipper 3, or neither, instead ending in a open tube. FIG. 5 represents an additional embodiment where a more rigid elastomer or polymer portion 13 is adhered to the standard adapter base 1. This portion contains threads 15 on the exterior surface. These threads lock together with a standard nipple locking annular clamp 14 that is common to traditional baby bottle design. This clamp ring contains internal threading 16 that engage the threading present on the adapter 15 in this embodiment. A standard infant drinking nipple 5 containing a hole for liquid withdrawal 22 is secured between the adapter 13 and the clamp 14 after tightening by pinching the rubber flange on the nipple 17 between the adapter and the flat portion on the annular clamp 18. The nipple top extends through a circular opening in the tightening clamp 26. A final embodiment of the invention shown in FIG. 6 represents the standard adapter 1 that contains a reinforced hard material shell 21 abutting the adjacent soft elastomer in the plug 8. A second manufactured portion 27 is snapped into the adapter base. The snap-in portion contains a hard material base 19 that when inserted into the adapter opening 11 presses on the base forcing the annular rings 9 securely against the neck of the bottle. The piece 27 is retained in place by a snapping mechanism where tabs 20 hold it against the adapter until these tabs are squeezed to remove this portion. The snap-in piece contains an internal passage 28 that allows for the removal of the liquid and the adapter may contain any of the above named fluid removal apparatuses including a standard nipple 2, sipper 3, straw 4, or screw on nipple 5.

While the preferred embodiments of the invention have been described above, it should be understood that changes in form, structure, arrangement, and practice that differ from those herein illustrated or detailed may be made within the underlying idea of the invention.

Breast milk is the ideal nutrition for the young infant because it provides advantages over infant milk formula in terms of general health, growth and development, while reducing the risk and/or severity of diseases, including diarrhea,¹⁻³ respiratory tract infection,^{4,5} urinary tract infection,⁶ otitis media,^{7,8} and necrotising enterocolitis.⁹

Working moms sometimes choose to pump and store breast milk to be offered to their children by their caregivers. Other mothers simply store milk to be offered to their babies when breastfeeding in public areas or in case of an occasional separation or to be given at a later time.

Breast milk handling and storage guidelines usually take into account the temperatures to which milk is submitted and these guidelines may change slightly from one source to the other⁸⁷⁻⁸⁹. Mothers usually don't have perfect control of room temperature when dealing with their milk and may face situations in which they are not sure if their milk is still good for human consumption. These situations include milk stored during power outage, presenting an unpleasant smell⁹⁰ or left in an unknown temperature for more than 6 hours (at home or on the go). Although milk can sometimes still be used to feed babies under these circumstances, the usual strategy is to discard the milk to avoid exposing the baby to food-borne illnesses.

Raw foods of animal origin, such as milk, frequently are contaminated with bacteria common in the food chain⁹¹. These microorganisms can replicate, and according to the type and amount of bacteria, cause fever, vomiting, diarrhea, and abdominal pain⁹¹. On the other hand, breast milk is a very precious liquid for mothers, and they are usually unwilling to discard it when it could still be in good condition. One alternative to increase the useful life of breast milk would be heating. However, high heating may change some nutrients in breast milk, including ascorbic acid (vitamin C) and some proteins⁹⁰.

Herein we describe a monitor or a device, e.g., a hand-held, fast, reliable monitor or device, for determining if spoilage has occurred. We also describe the kit and methods to prepare the monitor. As such parents and caregivers could diagnose spoilage of products and protect their babies from food-borne illnesses and, on the other hand, avoid disposing breast milk when it is still good for consumption.

Aspects disclosed herein relate to devices for determining if the mammalian breast milk has spoiled. Certain embodiments provide an apparatus, which comprises a detecting agent that makes use of the change in color observed when indicator molecules respond to a change in pH as a result of spoilage. Indicators are typically complex organic weak acids or weak bases comprising a UV, visible, or IR chromophore with an absorbance maximum that varies as a function of the pH of the environment. Such molecules are, independently for each occurrence, able to accept or to donate a proton, as represented by equilibrium equation (1), wherein a general indicator of the formula HX is ionized in solution:

HX [symbol] H⁺+X⁻ (1)

In certain embodiments, the detecting agent is used in conjunction with a base. Alternatively, the detecting agent is a small molecule or polymer which undergoes a color change in response to a change in oxidation state. In certain embodiments, the base can be added to breast milk at the same time as the detection agent or the base can be added first, followed by the detecting agent. In certain embodiments, the detecting agent is added first, followed by the base. In certain embodiments wherein the detecting agent is absorbed or covalently attached to a substrate, the base can be added to the breast milk and then the breast milk can become in contact with the substrate to afford a signal. In certain embodiments, the breast milk is passed though a resin or filter which is basic, followed by exposure to the detecting agent, which then affords a signal. The time of measurement is short, such that real time information can be obtained. More than one measurement may be made in a single day. In certain embodiments, molecules that undergo a change in their chemical structure so as to give a change in an electrochemical signal and/or response may also be used as detecting agents.

In the monitor device described above said detecting agent is selected from the group consisting of, but not limited to, litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino)azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R and salts thereof.

The FDA currently accepts two separate testing methodologies to ensure that a drug or device is free of endotoxin contamination. The first is by injecting a sample into a rabbit in vivo model to see if a fever develops. Unfortunately, nothing can be determined about the concentration of the endotoxin in the sample and so this technique, besides raising ethical concerns, does not provide any quantifiable information. The more recent test, approved for use in 1987, uses the lysate from a horseshoe crab amebocyte.³⁰ In the presence of endotoxin either on live, killed, or destroyed bacteria the limulus amebocyte lysate (LAL) will activate an enzymatic cascade. The cascade involves a two step process whereby the presence of an endotoxin catalyzes the conversion of a proenzyme into a coagulase enzyme. This enzyme in turn catalyzes the conversion of coagulogen into coagulin (FIG. 7). This reaction process is the basis for three separate LAL testing methodologies. The first technique measures turbidity development in a sample using either a kinetic or endpoint measurement. Coagulin is generally insoluble and by measuring the turbidity the quantity of the endotoxin can be determined. A second method using transmitted light measurements involves a modified synthetic substrate that is added to the LAL. Enzymatic activity on this substrate releases a chromogenic agent which will change the color of the solution. For example, p-nitroaniline can be cleaved from a peptide substrate in the process going from colorless to yellow. The two above named techniques both suffer from the drawbacks of requiring transmitted light through a sample to determine the endotoxin concentration. The opaque nature of milk makes this type of reading difficult and spotting a turbidity change would be next to impossible. Though reading a color change visually from colorless to yellow might be feasible, judging the color gradient would become difficult and differences in breast milk coloration between different mothers would only compound the problem.

The third LAL method, and the technique that we have chosen to use, is the method out of the three that is described in the United States Pharmacopoeia. The test relies on the observation that by varying the amount of LAL added to a sample there will be a point when there is enough LAL and endotoxin to promote the formation of a critical coagulin concentration, forming a gel. The readout to such a method involves reacting the components for a predetermined amount of time in an endotoxin free container, and then inverting the vial to determine if a sufficient gel has formed to resist flowing. In the reverse sense, if a constant amount of LAL is present and varied amounts of endotoxin are added, there exists a cutoff of endotoxin that amounts higher than this value will clot the mixture and those lower will not. By varying the amount of LAL added, a whole range of endotoxin concentrations can be assayed for. Besides not needing a transmitted light source, the technique produces binary results in the determination of what samples of milk would be safe to freeze for future distribution and which ones would be unsafe, with this cutoff being readily adjustable depending on the needs of the bank.

Current regulations on acceptable live bacteria counts in pasteurized banked milk varies depending on the country the milk bank resides in. For example, Scandinavian countries use a cutoff of 10,000 colony forming unites (cfu) per mL to be the tolerable limit.³² As noted earlier, endotoxin measurements are calculated in endotoxin units (EU) per a unit volume (EU/mL), so a conversion between these values needs to be done. Bacterium are generally accepted to have about 10̂6 endotoxin molecules, and 1 EU corresponds to 100 pg of endotoxin.^{23,24} So 10,000 cfu/mL would correspond to an endotoxin concentration of approximately 1 EU/mL. We have initially chosen to set our detection system to a more stringent 0.125 EU/mL to determine if a higher accuracy level can be achieved with breast milk. Again, this level is somewhat arbitrary and can be adjusted easily by adding or subtracting amounts of LAL in the mixture. This starting point shows the power of this technique and how it can easily detect bacteria counts as low as 1000 cfu/mL if not lower in the complex milk environment.

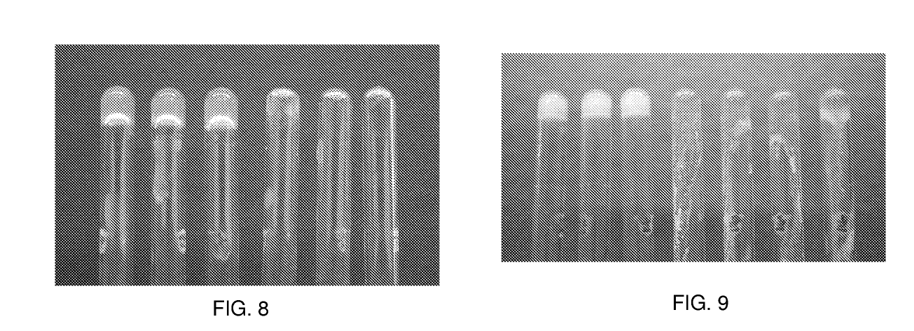

To examine the validity of the LAL test we began with sterile water samples doped with E. coli O55:B5 endotoxin (Lonza; Walkersville, Md.) which was reconstituted to a final concentration of 20 EU/mL. Limulus Amebocyte Lysate (LAL; Lonza) was dissolved in endotoxin free water to a final concentration of 42.9 mg/mL. This LAL amount was chosen so that the gel cutoff was achieved by an endotoxin level of 0.125 EU/mL. Samples of the same endotoxin free water were then doped with the E. coli endotoxin to achieve a logarithmically spaced concentration series of 0.5, 0.25, 0.125, 0.0625, 0.0312, and 0 EU/mL. The samples were mixed in an endotoxin free vial by combining 100 μL of the endotoxin samples and 100 μL LAL solution and incubated for 37° C. in a heating block for 60 minutes. After the incubation, the vials were inverted and photographed to determine whether the gel retained sufficient compositional strength to resist the gravitational forces. As can be seen in the photos the system functioned as expected where the high concentrations of 0.5, 0.25, and 0.125 were gelled while the lower concentrations of endotoxin free control flowed (FIG. 8).

After validating the proof-of-principle with water, we next examined whether detecting endotoxins in breast milk was feasible. Such a test has not been previously reported in human breast milk. As can be imagined, breast milk presents a complex environment in which to run this assay with a wide variety of salts, proteins, fats, and carbohydrates that could interfere with the gel formation.⁹² Additionally, LAL is sensitive to pH and must be run at pHs between 6.0 and 8.0 (per the manufacturer product manual). Fortunately, breast milk, though it does vary slightly in pH, is physiologically confined between a pH of 7.1 and 7.4 for all mothers negating this concern.⁹³ We began by testing the LAL method outlined above with 1 day old breast milk that had been refrigerated after collection. The milk was doped with varied levels of E. coli endotoxin to achieve final exogenous concentrations of 0.5, 0.25, 0.125, 0.0625, 0.0312, and 0 EU/mL of milk in addition to an additional control of undoped milk that would not have any LAL added to it to determine if the 37° C. heating process had any discernable effects. Next, 100 μL of the doped and undoped milk samples were added to a glass endotoxin free vial and were mixed with 100 μL of the LAL solution before incubation in a heating block for 1 hour at 37° C. After 1 hour, the tubes were inverted and photographed to examine for the presence of a formed gel (FIG. 9). As can clearly be seen in the image, the test kit performed identically to its performance in water gelling at concentrations of 0.125 EU/mL and greater as designed. The gel is easy to see using the naked eye and so presents a rapid and convenient method to examine for the presence of endotoxins.

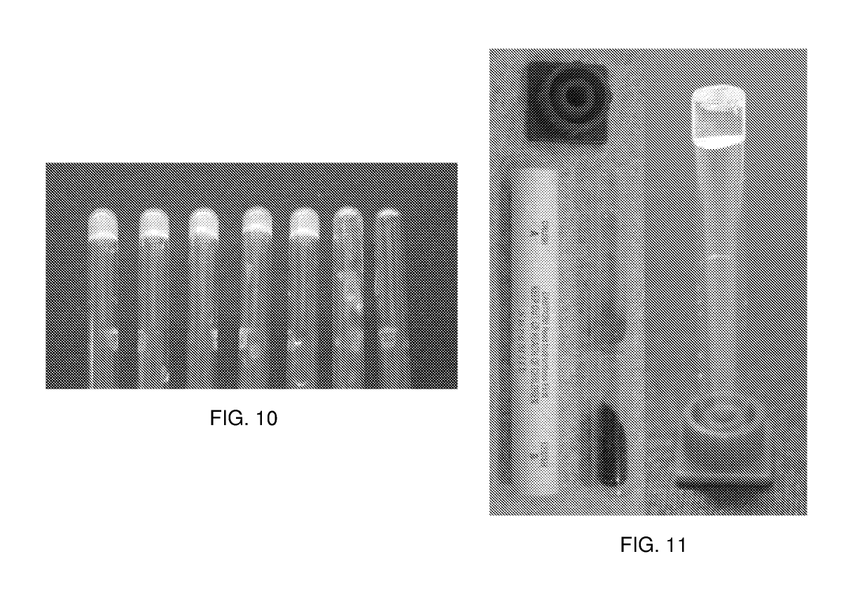

A second consideration of the kit would be in examining how stored milk would function in regards to gel formation. A sample of milk was collected and immediately frozen at −20° C. for 8 months. Upon thawing the milk was examined and found to form fat globules, which is not uncommon for prolonged milk storage.⁹⁴ To remove these globules the milk was briefly sieved through a filter. Next, the milk was again doped with the same exogenous endotoxin using identical concentrations as outlined above and the experiment was repeated by mixing the milk with the LAL reagent and incubating for 60 minutes. A water control was run in parallel to verify that the reagents functioned as expected. The inverted vials were photographed for the presence of the gel (FIG. 10). As can be observed, a shift occurred at what amount of doped endotoxin formed a gel. All doped endotoxin concentrations formed a gel, while the sample with 0 EU/mL did not. Clearly this sample did not endogenously possess sufficient endotoxin, however, a small exogenous addition of 0.0312 EU/mL was enough to tilt the balance. Therefore, we can conclude that this particular sample had an endogenous endotoxin concentration less than 0.125 but more than 0.0625 EU/mL. These experiments show the versatility of such a test, but again, the produced product will be designed with an inherent cutoff selected in line with the needs of the hospitals and milk banks. If a binary test were run on this sample with the cutoff being 0.125 EU/mL than this sample would have not gelled and would pass screening in regards to its endotoxin concentration. This prolonged storage for 8 months involved immediate freezing in dry ice after pumping and then careful temperature control at −20° C., a process that would be extremely difficult for mothers to replicate in their commercial freezers and so their samples will more readily promote endotoxin formation as has been noted in the literature.¹⁰ Milk donated to the banks kept at home for similar amounts of time would most likely not pass the screening.

The requirements for the device design are fourfold: 1) the reagents must not come into contact with the milk until the device is closed; 2) the method of delivering the milk and reagents into the sample must be straightforward and accurately controlled; 3) the entire device must be easy to operate; and 4) the results must be easy to read. To meet these requirements we have created a prototype comprised of a flexible vial with a cap holding two crushable glass ampoules. One ampoule will contain the LAL and the other, if needed, will contain a dye that will allow for easier visualization of the gel (FIG. 11). The vial will be wrapped in a removable paper sleeve to ensure that upon squeezing the closed vial to break the ampoules, no injuries to the user's fingers occur. We have tested crushing the ampoules over a hundred times and the glass puncturing the vial has yet to occur. Additionally, the cap used will contain a base upon which the inverted tube can be set for viewing. The user will be supplied with a sterilized syringe to accurately measure 100 μL of milk. The overall size of the device is rather small needing to be only 8.5 cm high and ¾ cm in diameter. The usage procedure will be: 1) 100 μL of milk is placed into the vial using the supplied syringe; 2) the cap is placed onto the vial sealing the chamber; 3) the user squeezes the tube at two locations to break the ampoules containing the LAL and dye, if needed, and shakes to mix; 4) the sleeve is removed and the tube is placed into a 37° C. heating block or water bath and left for 1 hour; 5) after 1 hour the device is retrieved, inverted, and set upright on the cap; 6) the user determines if a gel was formed and thereby determines the endotoxin safety of the milk sample either disposing of the original milk container or setting it aside for pasteurization; and 7) the sealed container is disposed of without reopening the cap. If an elevated level of endotoxin is reported, the user will be advised to retest to ensure the result was not a false positive. Additionally, positive controls will be supplied with the kit with ampoules containing the LAL and a sample of lyophilized endotoxin adjusted to just above the cutoff point. This control test can be run to ensure that the LAL is functioning correctly and would rule out the occurrence of any false positives or false negatives.

Herein, we describe a monitor or a device, e.g., a hand-held, fast, reliable monitor or device, for determining if excess endotoxins are present. We also describe the kit and methods to prepare the monitor. As such parents caregivers, or technicians could diagnose contamination of products and protect infants from food-borne illnesses and, on the other hand, avoid disposing breast milk when it is still good for consumption.

A typical sample of human mother's milk can contain anywhere between 1 to about 18% fat. A fat content of 5 wt % is considered normal or ideal and, in fact, this is the concentration of fat in milk supplements. The fat constituent of breast milk is the glycerol based lipids which are composed of many types of fatty acids. These fatty acids include but are not limited to: 10:0, 12:0, 13:0, 14:0, 14:1w5, 15:0, 16:0, 16:1w7. 16:2w7/17:0, 18:0, 18;1w9, 19:0, 18:2w6, 18:03w6/20:0, 18:3w3/20:1, 21:0, 20:2w6, 20:3w6, 20:4w6, 24:0, 22:4w6, 22:5w6, 22:5w3, and 22:6w3.

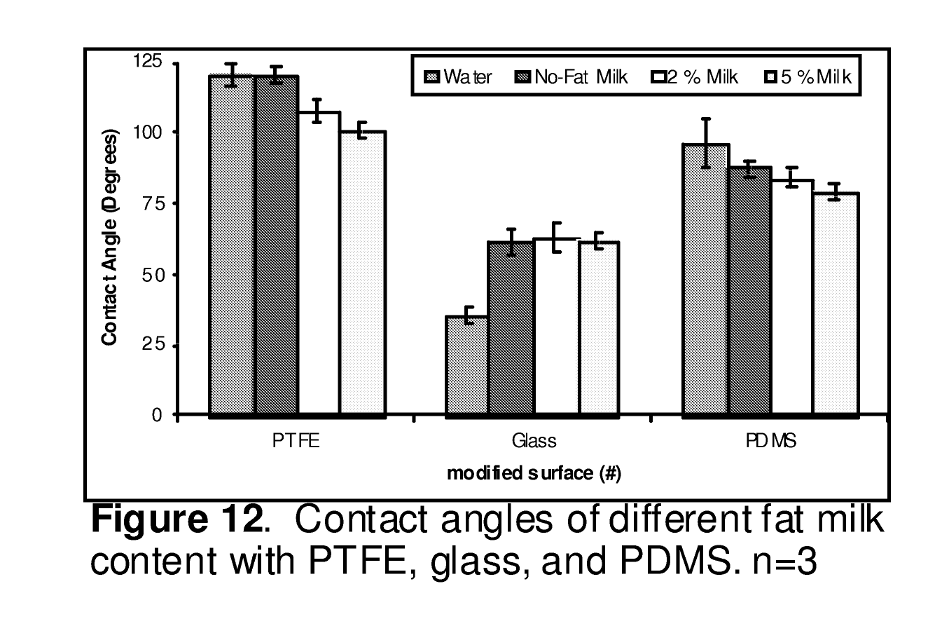

Embodiments of the caloric monitor are based, at least in part, on the principles of surface tension forces and surface free energy. These principles can be used to vary the interaction of a liquid with a surface, and this was elegantly demonstrated a few years ago when Chaudhury and Whitesides reported how to make water run uphill.⁹⁵ We are using surface tension principles to detect the changes in breast milk fat content with our monitor. Specifically, the monitor relies upon the change in hydrophobicity of the breast milk sample, which is directly related to the fat concentration. Breast milk containing 2% vs. 10% w/v fat will interact differently with a surface. The type of surface (more or less hydrophobic) and the size and shape of a drop of a liquid can affect the interaction between the surface and the drop. For example, a drop of water will minimize its contact with a hydrophobic surface by increasing the contact angle. In our first experiment, we measured the contact angle of no-fat, 2%, and 5% milk on three common surfaces—PTFE (polytetrafluoroethylene), glass, and PDMS (polydimethylsiloxane). As shown in FIG. 12, a trend can be observed between the fat content and contact angle on the two hydrophobic surfaces, however, on the glass surface no such dependence was observed. Polymers suitable for use include, but are not limited to, Teflon, polystyrene, modified polystyrene, polypropylene, polyurethane, ethylene vinyl alcohol, (E/VAL), fluoroplastics, (PTFE), (FEP, PFA, CTFE, ECTFE, ETFE, polyacrylates, (Acrylic). polybutadiene, (PBD), polybutylene, (PB), polyethylene, (PE), polyethylenechlorinates, (PEC), polymethylpentene, (PMP), polypropylene, (PP), polyvinylchloride, (PVC), polyvinylidene chloride, (PVDC), acrylonitrile butadiene styrene, (ABS), Polyamide, (PA), (Nylon), polyamide-imide, (PAI), polyaryletherketone, (PAEK), (Ketone), polycarbonate, (PC), Polyektone, (PK), polyester, polyetheretherketone, (PEEK), polyetherimide, (PEI), polyethersulfone, (PES), polyimide, (PI), polyphenylene oxide, (PPO), polyphenylene sulfide, (PPS), polyphthalamide, (PTA), polysulfone, (PSU), allyl resin, (Allyl), melamine formaldehyde, (MF), phenol-formaldehyde plastic, (PF), (Phenolic), polyester, polyimide, (PI), silicone, (SI).

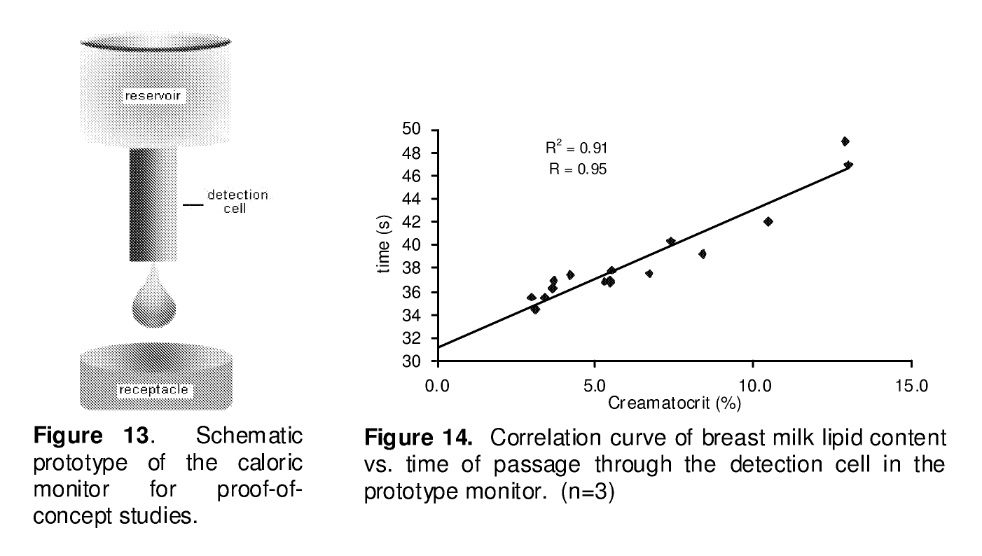

Building on these results, we performed additional studies with different surface compositions and chemistries, and then optimized the surface so as to obtain maximal differences in the contact angle with breast milk of varying fat content. Through this work, we have identified a surface composition to be used which is based on modified polystyrene or PTFE. The basic design of the monitor is shown in FIG. 13. The monitor consists of a reservoir for holding the breast milk sample, a detection cell that has a specific surface for interacting with the breast milk sample, and a receptacle for collecting the breast milk. As the milk passes through the detection cell, which is composed of the modified polystyrene, its rate of passage is dependent on its fat content. As such we can measure the time necessary for breast milk to flow through the detection cell and can correlate this to a specific fat content of the breast milk. Using this technology, we can quickly measure the fat/caloric content of breast milk using small volume samples (<1 mL).

The accuracy of the technology was determined using breast milk samples from four voluntary donors (with multiple samples from each donor). The fat concentration of the breast milk was obtained using Creamatocrit Plus™ (Medela). For our monitor, we measured the average time for 40 drops to pass through the detection cell. A correlation of R=0.95 was obtained using this laboratory prototype monitor (FIG. 14). The correlation between our device and the fat concentration is good, even though we are limited by the error on the x-axis generated by measurements taken from the Creamatocrit plus™ and by the error on the y-axis generated by the laboratory prototype which uses manual timing of the drop speeds. Currently, we are using a human-operated timer and a hand-made detection cell which generates a slight variability on each data point (±0.5 s). The use of a simple electronic counter instead of a manual counter to determine the time necessary for the drop to flow through the detection cell will improve this measurement. Similar counters are well known and heavily used as intravenous drop counters in hospitals and for chemical titrations and chromatography. These counters are easy to manufacture and inexpensive.

Mercury's affinity for proteins, and particularly the cysteine residues of these proteins, is well understood and generally regarded as the method through which mercury poisoning proceeds.⁹⁶⁻⁹⁸ Upon mercury binding, an enzyme will lose some of its potency, reducing its effectiveness in catalyzing the conversion of the substrate into the desired product. Laboratories have previously taken advantage of this affinity to develop enzymatic assays whereby the amount of product produced by the enzyme is calculated and used to indirectly determine the amount of mercury in a sample. The various enzymes previously examined include: mercuric reductase,⁹⁹ l-lactate dehydrogenase,¹⁰⁰ peroxidase,^{101,102} invertase,¹⁰³ δ-aminolevulinate dehydrogenase,^{104,105} and urease.¹⁰⁶⁻¹⁰⁹ Many of these enzymes produce products that would require complicated equipment to determine the results such as invertase which is so named because it converts sucrose to fructose in solution, thereby changing the polarization of transmitted light. An ideal readout for a personal mercury tester would be colorimetric.

With these ideas in mind we quickly settled upon the use of urease for four reasons. First, urease has been shown to be sensitive to Hg and insensitive to other heavy metal ions such as cadmium, lead, zinc, and nickel.¹⁰⁹ Secondly, mercury can affect the activity of the enzyme at concentrations down to 1 ppb. Thirdly, urease catalyzes the conversion of urea into carbon dioxide and ammonia [(NH₂)₂CO+H₂O→CO₂+2NH₃] and thus in aqueous solution increases the pH. Fourthly, urea is a stable enzyme that is not denatured until a temperature of 72° C. is reached and is readily stored lyophilized for two years at 4° C., indicative of good shipment and storage characteristics.¹¹⁰

The change of pH generated by the enzyme can be readily detected using an appropriate pH indicating dye. A device/monitor is described that comprises a flexible tube containing two crushable glass ampoules. One ampoule contains powdered urease enzyme while the second contains the pH dye and the urea substrate. The mother will place a small sample of her breast milk (1 mL) into the tubing, close the cap and then squeeze the vial, resulting in the breakage of the ampoules and the release of the urease, dye, and urea. As the pH of the milk increases, the color of the dye changes to indicate the enzyme activity and after a predetermined wait, the amount of mercury in the milk can be read against a printed gradient. Milk that contains differing amounts of mercury results in different final colorations. As described below, we have shown that a monitor based on these principles can be created for mercury concentration detection in solutions as diverse as water and infant formula. We have also shown that the color change in formula is sensitive to parts-per-billion of mercury alone and not competing ions of iron, copper, manganese, zinc, and various other metallic ions which are all present in infant formula in concentrations a thousand times greater than mercury (diluted formula produces similar results).

Urease is an active enzyme with a high activity unit per mg of powder. Consequently, a very small amount of enzyme is capable of catalyzing the conversion of a large amount of urea into ammonium ions. Both water and formula have neutral pH values of 7, but the introduction of the components would rapidly drive this value into the basic regime, but because of the weak alkalinity of the ion (K_{b}=1.78×10⁻⁵) the reaction would hypothetically terminate around a pH of 10. For this Hg monitor device the dye or detecting agent is selected from the group consisting of, but not limited to, litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino)azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarin Yellow R and salts thereof.

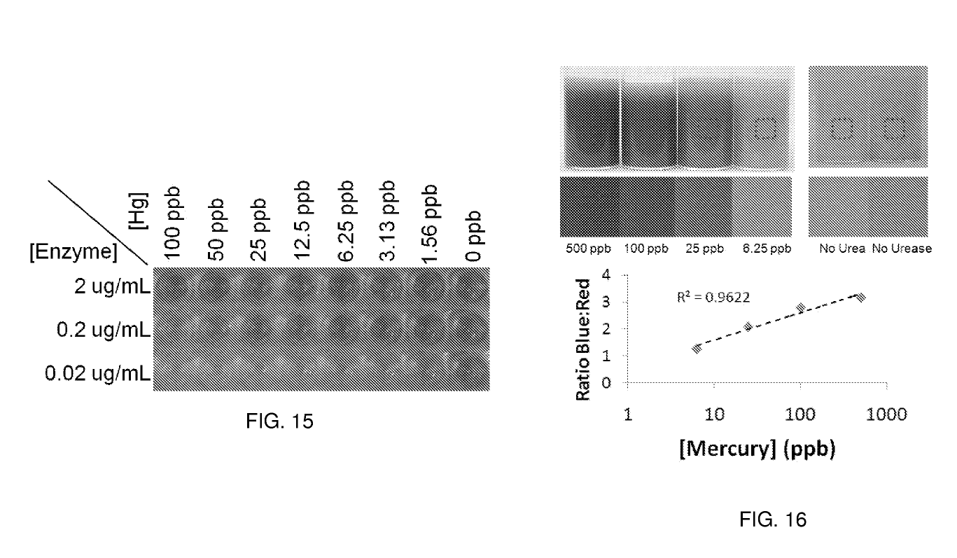

The dye bromothymol blue and dye combinations of bromothymol blue, phenolphthalein, methyl red, and thymol blue produced optimal results in infant formula and it was found that the solution is colored yellow at neutral pH but transitions to green and finally a blue/indigo coloration upon enzyme activity. Because formula is a buffered solution, as is breast milk, we elected to begin testing of the method using a 96-well plate format on non-buffered water. Mercury(II) trifluoroacetate (Sigma) was dissolved in nanopure water (17.9 MΩ-cm) to produce a stock solution of 2000 ppb (μg Hg/L) mercury ions. The stock concentration of mercury was diluted to produce a physiological logarithmic range of values from 1.56 to 100 ppb (7 points total). Additionally, control values of 0 and 1000 ppb were also included. Various concentrations of enzyme (Urease Type III from Jack Bean, Sigma) were added to the wells along with the dye mixture and the plates were let stand for 10 minutes at room temperature (RT). Urea (Sigma) was added in excess so that it would not affect the kinetics of the experiment and the color change was recorded over time with a camera (Canon EOS Digital Rebel). The reaction proceeded as expected with higher amounts of mercury and lower enzyme concentrations taking longer to produce a color change (FIG. 15). As expected, higher concentrations of mercury took overnight to fully reach a final coloration showing that the urease isn't denatured permanently. Wells with no mercury changed the fastest as there was no enzyme inactivation and control wells that contained all components except for the enzyme or the substrate never changed color proving that the change was not due to a specific component alone but required the combination of mercury, dye, urease, and urea.

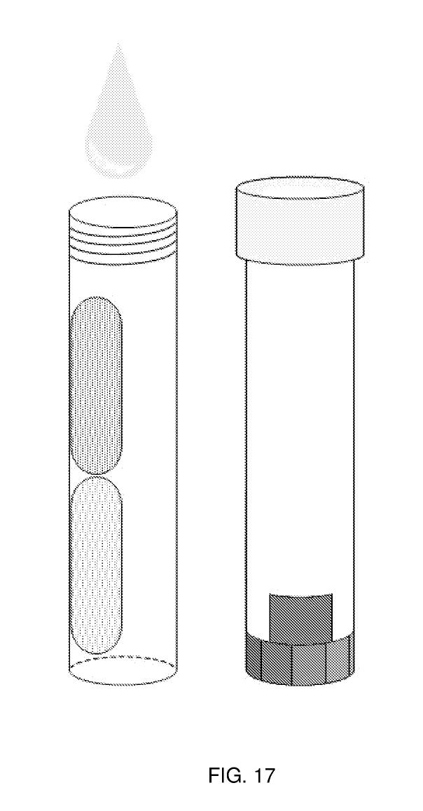

Prior to working with breast milk we have performed studies with infant formula because it is consistent between doses, more readily available, and contains a minimal amount of endogenous mercury (with breast milk this amount would be unknown). Formula presents a more complicated environment in which to test the assay than what is provided in water. Infant formula is not only buffered, but contains a myriad of proteins with which the metal ions could also interact. Infant formula (Nestlé) was mixed with nanopure water according to manufacturer directions. This was followed by the addition of varied amounts of mercury, the dye solution, urease, and finally urea producing a final volume of 3 mL. After overnight room temperature reaction to ensure complete color development, the results showed a consistent color gradient that depended on the amount of mercury present (FIG. 16; top). Again, controls without either enzyme or substrate did not produce a color change and these controls remained the neutral yellow color for the course of the experiment (FIG. 16; top). Taking the ratio of the blue:red coloration in the pixels contained in the red dashed boxes in the figure (and blown up immediately below), there is a clear linear trend dependent on Hg concentration (R²=0.96; FIG. 16; bottom). This verifies the use of the enzymatic reaction to calculate the amount of mercury at physiological concentrations.

Formula contains a variety of metals at concentrations many times above that of the mercury (Table 2) and the recorded color gradient was insensitive to these ions, diluted formula samples containing less of these ions still produced a color gradient. Final colorations are currently achieved after a few hours in solution, but by varying the relative amounts of dye/urease/urea we have shown in both water and formula that quicker or slower timings can easily be achieved.

The desirable features for the device design are fourfold: 1) the reagents desirably does not come into contact with the milk until the device is closed; 2) the method of delivering the milk and reagents into the sample is desirably straightforward and accurately controlled; 3) the entire device is easy to operate; and 4) the results are desirably easy to read. To meet these requirements we will use a flexible vial with a screw cap holding two crushable glass ampoules with one containing the enzyme and the other containing the dye and substrate (FIG. 17). The entire vial will be wrapped in a paper sleeve to ensure that upon squeezing the closed vial to break the ampoules, no injuries to the user's fingers occur. A number of other combinations are possible using this design. Additionally, this paper sleeve will have a small opening through which the final color can be read and the litmus-type scale will be printed below this window to allow the user to match the developed color with the closest match on the gradient scale. An outcomes table will be supplied with the product which will convert the color to the US ASTDR mercury recommendation for infants of a particular weight. There may be 7 colors in the gradient scale that will represent values over the relevant physiological mercury concentrations (0-25 ppb) and each color may have a specific number assigned to it for ease of use and recall. The user will be supplied with a sterilized syringe, which mothers are already familiar with, to accurately measure 1 mL of milk. The overall size of the device is rather small needing to be only 8 cm high and 1 cm in diameter. The usage procedure will follow the following steps: 1) 1 mL of milk is placed into the vial using the supplied syringe; 2) the cap is screwed onto the vial sealing the chamber; 3) the user squeezes the tube at two locations to break the ampoules and shakes to mix; 4) the device is set aside for a predetermined amount of time to ensure final coloration is reached; 5) rotating the sleeve to move the window the user finds the gradient color that best matches the developed color; 6) the user uses the supplied outcome table to determine the safety of their milk in regards to the governmental recommendation;⁵⁷ and 7) the sealed container is disposed of without reopening the cap. If an elevated level of mercury is reported, the user will be advised to retest to ensure the result was not a false positive, followed by consultation with their health care provider to discuss the results. This recommendation level will be set according to the guidelines for mercury levels in milk according to the ATSDR of 2 μg Hg/kg/day.

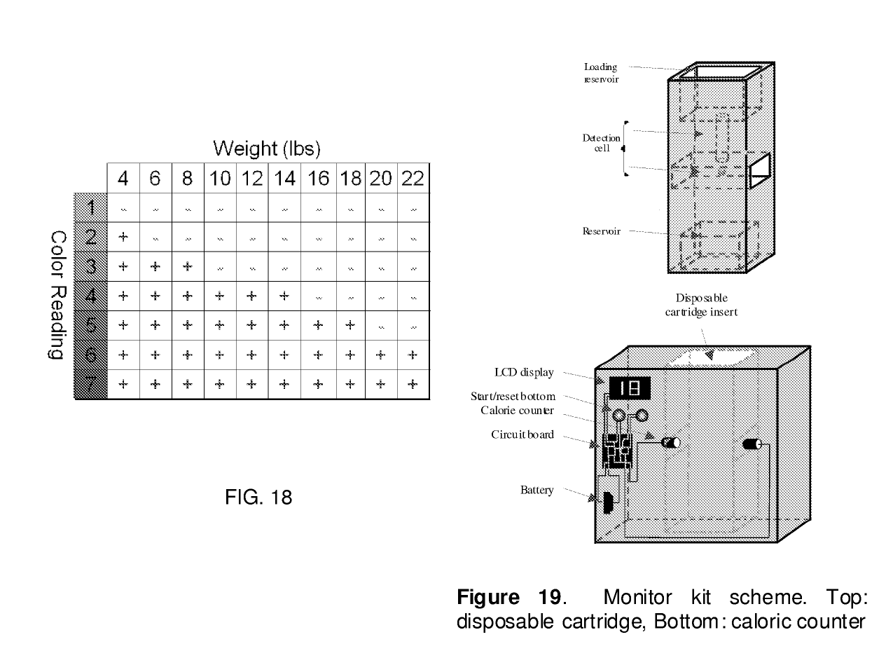

Because the weight of the infant is important in determining the tolerable mercury intake, the outcome regarding breast milk concentrations that are “safe” and those that the mother should talk to her health care professional will vary depending on the child's size. To solve this problem, each kit will contain a sliding chart that the mother will adjust so that the weight of her child is visible as the selectable criteria. Next, she will take the color reading from the device and use this to index the recommendation on the table regarding her mercury concentration (FIG. 18). As an example, a breast milk concentration of 5 μg Hg/kg of milk is considered “safe” for children over 5.5 pounds, but not those under and the chart will reflect this reality. The gradient scale will be set according to the color values obtained and adjusted in Aim 1 below so as to provide the maximum amount of information to the mother. Those mothers who have initial mercury readings above the recommended level for their child will be encouraged to repeat the readings to ensure that the initial measurement was not a false positive and then to consult their health care provider.

Methods of the Invention

Certain embodiments disclosed herein relate to monitoring the calorie content of breast milk as a function of daily eating habits and food consumption in order to optimize the number of calories in breast milk. Certain aspects further provide for a process or method of measuring the calorie content in breast milk either before or after consuming a meal, feeding an infant/newborn, and repeating this procedure such that good nutritional behavior is adopted. A closed-looped system is useful to monitoring and controlling the calorie content of milk

Another process described herein is the use of said device described to detect the concentration of heavy metals such as Hg in breast milk and then alter the mother's feeding habits to reduce the concentration of heavy metal in her breast milk. The mother can eliminate or reduce her consumption of fish. Alternatively, the mother can stop breast feeding and provide formula milk to the infant. A closed-looped system is useful to monitoring and controlling the Hg or other heavy metal present (e.g., Pb) content of milk.

Sterilization Procedures

Procedures are known in the art for sterilizing a device, chemical composition, or package. As such the monitors and devices disclosed herein can be sterilized either separately or as a kit. Sterilization may be accomplished by chemical, physical, or irradiation techniques. Chemical methods include exposure to ethylene oxide or hydrogen peroxide vapor. Examples of physical methods include sterilization by heat (dry or moist), retort canning, and filtration. The British Pharmacopoeia recommends heating at a minimum of 160° C. for not less than 2 hours, a minimum of 170° C. for not less than 1 hour and a minimum of 180° C. for not less than 30 minutes for effective sterilization. For examples of heat sterilization, see U.S. Pat. No. 6,136,326, which is hereby incorporated herein by reference. Passing the chemical composition through a membrane can be used to sterilize a composition. For example, the composition is filtered through a small pore filter such as a 0.22 micron filter which comprises material inert to the composition being filtered. In certain instances, the filtration is conducted in a Class 100,000 or better clean room.

Irradiation methods include gamma irradiation, electron beam irradiation, microwave irradiation, and irradiation using visible light. One preferred method is electron beam irradiation, as described in U.S. Pat. Nos. 6,743,858; 6,248,800; and 6,143,805, each of which is hereby incorporated herein by reference. There are several sources for electron beam irradiation. The two main groups of electron beam accelerators are: (1) a Dynamitron, which uses an insulated core transformer, and (2) radio frequency (RF) linear accelerators (linacs). The Dynamitron is a particle accelerator (4.5 MeV) designed to impart energy to electrons. The high energy electrons are generated and accelerated by the electrostatic fields of the accelerator electrodes arranged within the length of the glass insulated beam tube (acceleration tube). These electrons, traveling through an extension of the evacuation beam tube and beam transport (drift pipe) are subjected to a magnet deflection system in order to produce a “scanned” beam, prior to leaving the vacuum enclosure through a beam window.

The dose can be adjusted with the control of the percent scan, the beam current, and the conveyor speed. In certain instances, the electron-beam radiation employed may be maintained at an initial fluence of at least about 2 mCurie/cm², at least about 5 mCurie/cm², at least about 8 mCurie/cm², or at least about 10 mCurie/cm2. In certain instances, the electron-beam radiation employed has an initial fluence of from about 2 to about 25 mCurie/cm². In certain instances, the electron-beam dosage is from about 5 to 50 kGray, or from about 15 to about 20 kGray with the specific dosage being selected relative to the density of material being subjected to electron-beam radiation as well as the amount of bioburden estimated to be therein. Such factors are well within the skill of the art, given the benefit of this disclosure.

The composition to be sterilized may be in any type of container that is at least partially permeable to election beam, such as glass or plastic. In certain embodiments, the container may be sealed or have an opening. Examples of glass containers include ampoules, vials, syringes, pipettes, applicators, and the like. The penetration of electron beam irradiation is a function of the packaging. If there is not enough penetration from the side of a stationary electron beam, the container may be flipped or rotated to achieve adequate penetration. Alternatively, the electron beam source can be moved about a stationary package. In order to determine the dose distribution and dose penetration in product load, a dose map can be performed. This will identify the minimum and maximum dose zone within a product.

Procedures for sterilization using visible light are described in U.S. Pat. No. 6,579,916, which is hereby incorporated by reference. The visible light for sterilization can be generated using any conventional generator of sufficient power and breadth of wavelength to effect sterilization. Generators are commercially available under the tradename PureBright® in-line sterilization systems from PurePulse Technologies, Inc. 4241 Ponderosa Ave, San Diego, Calif. 92123, USA. The PureBright® in-line sterilization system employs visible light to sterilize clear liquids at an intensity approximately 90000 times greater than surface sunlight. If the amount of UV light penetration is of concern, conventional UV absorbing materials can be used to filter out the UV light.

As discussed above, in certain embodiments, one or more of the compositions, reagents, or components of a kit has been sterilized. The sterilization may be achieved using gamma radiation, e-beam radiation, dry heat sterilization, ethylene oxide sterilization, or a combination of any of them. In certain embodiments, compositions disclosed herein may be sterilized to provide a Sterility Assurance Level (SAL) of at least about 10⁻³. The Sterility Assurance Level measurement standard is described, for example, in ISO/CD 14937, the entire disclosure of which is incorporated herein by reference. In certain embodiments, the Sterility Assurance Level may be at least about 10⁻⁴, at least about 10⁻⁵, or at least about 10⁻⁶.

Vessels, Delivery Systems, and Devices

Certain embodiments of the spoilage and heavy metal detection and calorie monitor systems described herein advantageously utilize breast milk that contacts a detecting agent. Consequently, the breast milk sample must be added to a vessel for the subsequent reaction and analysis. The sample can be delivered for analysis using a large number of delivery devices. For example, the delivery system may be capillary tube, pipette, spoon, “eye dropper,” or syringe. The analysis can occur in a single or multiple vial, cup, mug, ample, chamber, container, tube, beaker, goblet, reservoir, microarray, or nanoarray, which may be optically clear. The contents of the single or multiple vial, cup, mug, chamber, container, beaker, goblet, reservoir are mixed via hand shaking, motor, vortexing, or push and pull of a syringe. Alternatively, a mixing chamber may be advantageous since the components can be separately flowed or flowed together for analysis.

In certain embodiments, the detection agent is absorbed to the single or multiple vial, cup, mug, chamber, container, beaker, goblet, reservoir, paper, fabric, or microarray. In certain embodiments, the detection agent and/or base and/or enzyme are absorbed to the single or multiple vial, cup, mug, chamber, container, beaker, goblet, reservoir, paper, fabric, or microarray. In certain embodiments, the detecting agent is covalently attached to the single or multiple vial, cup, mug, chamber, container, beaker, goblet, reservoir, paper, fabric, or microarray. In certain embodiments, the detecting agent and/or base and/or enzyme are covalently attached to the single or multiple vial, cup, mug, chamber, container, beaker, goblet, reservoir, paper, fabric, or microarray. Covalent attachment chemistry is well known in the art.

A further embodiment provided herein is the use of one or more crushable ampoule(s) (glass or plastic) housed in a plastic container (tube, bottle, syringe,) whereby the ampoule contains the detection agent and the base or enzyme/substrate. The detection and base or enzyme/substrate can be in the sample ampoule or they can be in separate ampoule. Alternatively, the base or enzyme/substrate can be in an ampoule and the detection agent can be in the plastic container or vice-verse. Upon addition of the breast milk to the plastic container, the ampoule(s) is crushed and the detection process begins. The detection agent then undergoes a change or signifies a change—such as a high concentration of Hg in the breast milk or that the breast milk has spoiled. This change can be a color change, a conductivity change, a precipitation, or polarization change.

In certain embodiments of the kits, a liquid reagent is contained in a vial, and is contained in a single-barreled syringe. At time of use, the vial and syringe are placed into liquid communication, and the liquid is withdrawn from the vial into a filled syringe of milk, thereby mixing the components.

The calorie monitor consists of two parts: caloric counter and disposable cartridge. The caloric counter and the disposable cartridge will be produced using one of a variety of manufacturing methods including injection molding.

The overall counter has H×W×D dimensions, for example, of 60, 30, and 60 mm, respectively. The disposable cartridge slides into the caloric counter for the reading of the fat/caloric content (FIG. 19). The cartridge has a top chamber into which the breast milk is placed, a detection cell which the breast milk runs through, and a receptacle at the bottom for collecting the breast milk after the measurement. The cartridge will be made from polycarbonate plastic. Polycarbonate is a transparent thermoplastic with relatively high heat resistance and low water absorption. Polycarbonate was chosen to preserve our ability to use several rapid prototyping methods. Moreover, polycarbonate is easily machined using milling techniques, laser micromaching, hot embossing and injection molding. This flexibility is essential as we iterate through design changes. The detection cell, which is a 2 mm diameter tube, will be press-fit in the cartridge, where the outer diameter of the tube is slightly larger than the diameter of the part it has to fit into, so that the stress in the tube keeps it in place and sealed. This tube is constructed of Teflon, PDMA, polystyrene or other hydrophobic polymer or hydrophobically modified surface.

The caloric counter has an integrated circuit for measuring the time necessary for a drop to flow through the detection cell. A diode readout will report a number which will be correlated to caloric content. The counter component must be inexpensive to produce, but must also have good dimensional stability and toughness to maintain the alignment of the internal electronics necessary for repeatable measurements. An injection molding grade of acrylonitrile butadiene styrene, (ABS; chemical formula (C₈H₈.C₄H₆.C₃H₃N)ₙ), will be used to fabricate the counter. ABS is commonly used for injection molded parts; it is also recyclable. The counter will consist of two separately molded units which will be snapped together once the integrated circuit components are inserted.

Kits

In certain embodiments, kits are provided for conveniently and effectively implementing the methods associated with the devices disclosed herein. These kits house bottle adapters, spoilage, Hg, or caloric monitors. Such kits comprise any of the devices disclosed herein or a combination thereof, and a means for facilitating their use consistent with methods provided herein. Such kits provide a convenient and effective means for assuring that the methods are practiced in an effective manner. The compliance means of such kits includes any means which facilitates practicing a method described herein. Such compliance means include instructions, packaging, and dispensing means, and combinations thereof. Kit components may be packaged for either manual or partially or wholly automated practice of the foregoing methods. In other embodiments, embodiments disclosed herein contemplate a kit including devices described herein, and optionally instructions for their use. In certain embodiments, the compositions of detecting agents and base or enzyme/substrate of such a kit are contained in one or more vials, a compressible plastic or metal tube (for example, akin to a conventional toothpaste tube), or a packet that may be torn open.

In certain embodiments, the present technology relates to the aforementioned kit, further comprising a moisture-barrier element. The moisture-barrier element may be conditioned for use in the preparation of a solution to be used in a method according to certain embodiments. In certain embodiments, a second component of the kit may be contained within the moisture-barrier element. For example, one of the detecting agents, enzymes, or plastic parts may be contained in a moisture-barrier element, thereby limiting or preventing reaction with water. Further, a kit may contain a plurality of moisture-barrier elements, each of which may be conditioned for use in the same or distinct ways. For example, for a kit containing a plurality of water-reacting compounds, each may be contained in an individual moisture-barrier element. Alternatively, a moisture-barrier element may contain a plurality of water reacting reagents. A moisture-barrier element may be characterized in a number of ways or a combination thereof. For example, a moisture-barrier element may be characterized by its shape (e.g., pouch, vial, sachet, ampoule); composition (e.g., glass, foil, Teflon®, stainless steel); and/or it may be characterized by a functional quality (e.g., moisture-vapor transmission rate (MVTR)). MVTR is an important means of characterizing a moisture-barrier element because: those of ordinary skill in the art understand how to measure the MVTR of a material; MVTR values for various materials are known; and the MVTR of a moisture-barrier element quantifies its ability to exclude water from it contents.

Aspects disclosed herein also relate to provision of the aforementioned kit, which is portable and can be used indoors or outdoors including in the clinic, home, farm, zoo, or outdoors.

### Exemplification

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the present co-inventors. In light of the present disclosure and the general level of skill in the art, those of skill will appreciate that the following Examples are intended to be exemplary only and that numerous changes, modification, and alterations can be employed without departing from the scope of the invention.

### Example 1

### Creation of an Adapter Mold

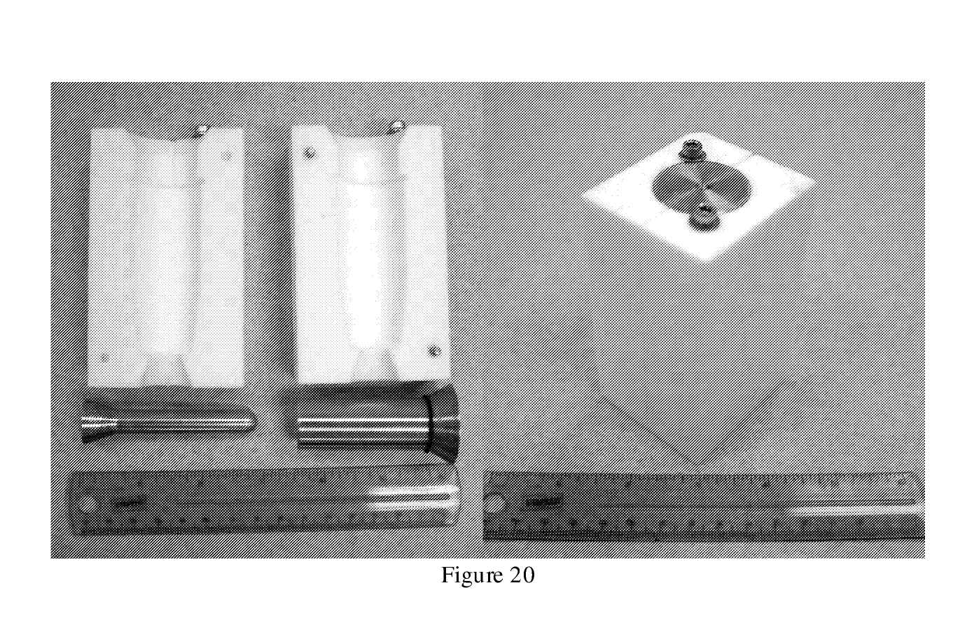

A mold that could be used to create the elastomeric bottle adapter was created by machining a slab of fluoropolymer (Teflon™) in combination with a lathed brass rod. The mold was created in four separate pieces (2 fluoropolymer, 2 brass) that when united created a negative space of the adapter design (FIG. 20). This mold could be filled with any curable liquid such as rubbers, latex, polymers, plastics, elastomers, molten metals, molten ceramics, molten glass, or waxes that when cured following the manufacturer's instructions would set into the shape of the negative space. The mold could then be deconstructed and the adapter removed.

### Example 2

### Unitary Silicone-Nipple Bottle Adapter

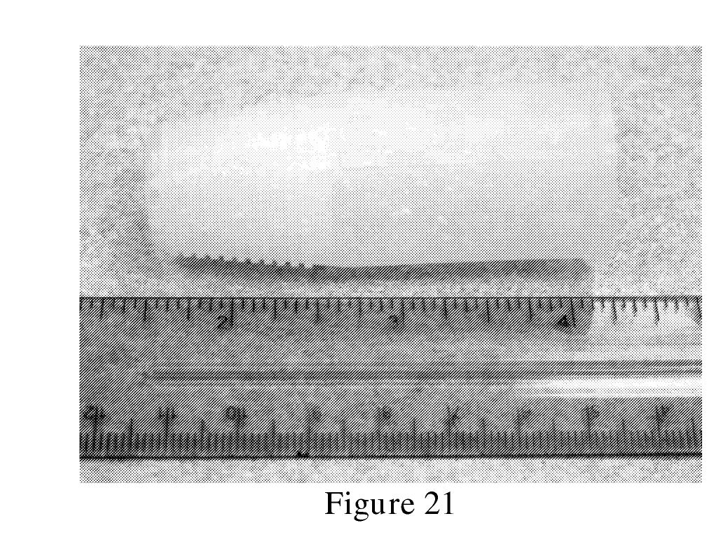

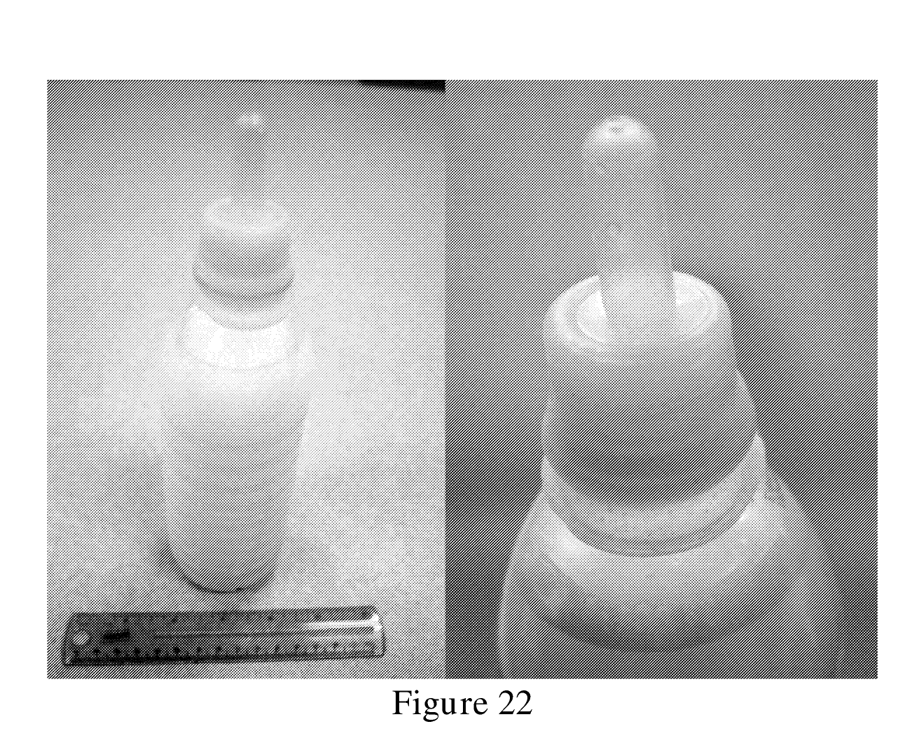

An adapter was created with a flexible silicone elastomer that was unitarily constructed with an internal tapered plug, external sealing flange, and an infant drinking nipple. The mold described above was filled with a prototypical silicone elastomer with platinum curative and cured following the manufacturer's instructions by leaving at room temperature overnight. After curing the mold was opened and the adapter was removed (FIG. 21). The construction of the entire resulting apparatus was made from a single elastomer piece with a resulting hardness of 10 on the Shore A durometer scale. The adapter could then be inserted and placed into a bottle to dispense a contained liquid through the nipple apparatus (FIG. 22). The liquid does not leak when the bottle is inverted and the strength of the adapter attachment to the bottle neck resisted removal forces (FIG. 23).

### Example 3

### Adapter Containing a Plurality of Silicone Elastomers

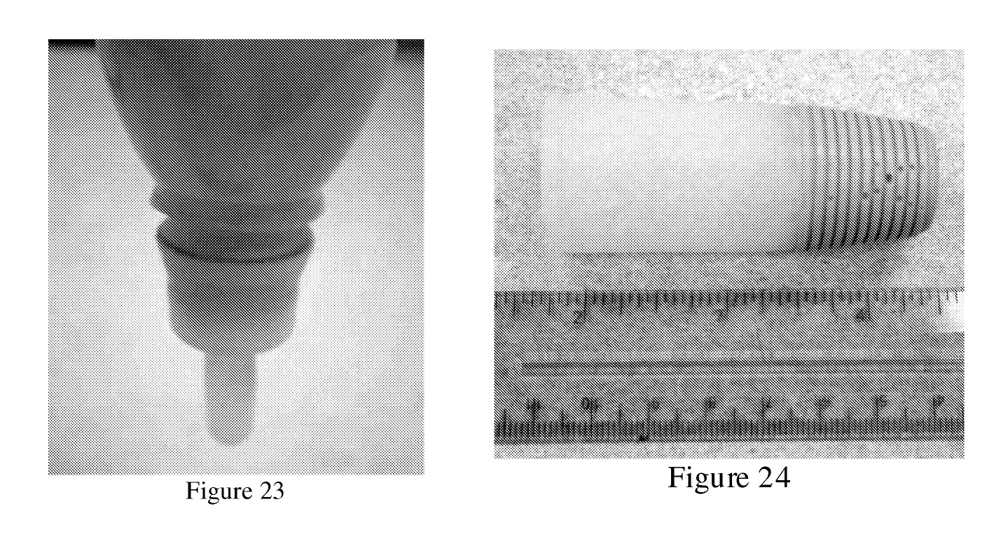

A second adapter was created by mixing two elastomers of different final cured hardnesses. The mold was again used as before, however, a platinum cured silicone elastomer with a final hardness of 30 on the shore A scale was poured first so that it would become the tapered plug portion of the adapter. This elastomer contained a blue dye to allow for easy visualization. A second elastomer with a final hardness of 10 was poured next to create the nipple and external flange portions of the adapter. The adapter was cured at room temperature overnight and the adapter was demolded. A clear separation with minimal mixing between the layers was observed (FIG. 24). In this manner, adapters containing a plurality of materials with different properties can be created and molded into a single unitary piece.

### Example 4

### Spoilage Detection

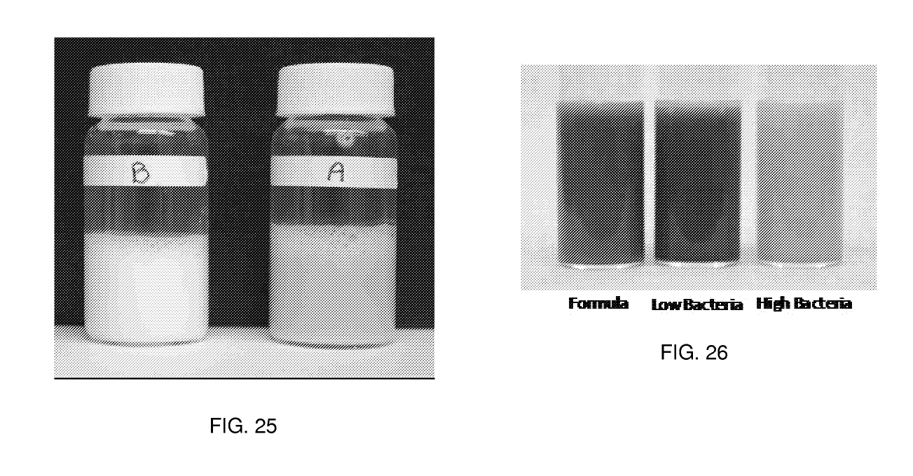

A solution of sodium hydroxide containing 1% of phenolphthalein at 1% in ethanol was prepared to afford a change in the phenolphthalein color at 8° Dornic acidity. 1 mL of this solution was introduced into a 5 mL vial. The vial can be used immediately with the liquid mixture, or after the added solution has evaporated and dried. Next, 1 mL of milk at different freshness ranging from 1 to 15° Dornic acidity was then added into this vial. The vials were then closed with a stopper or screw cap and shaken for 20 s. The vials containing less than 8° Dornic acidity show pink color. When the Dornic acidity is greater than or equal to 8°, the reaction between the milk, base, and indicator is incomplete and the phenolphthalein turns colorless. This change in color (going from pink to colorless) indicates that the milk has spoiled (See FIG. 25).

### Example 5

### Spoilage Detection

A kit is prepared as follows. The sodium hydroxide/phenolphthalein solution is introduced into a translucent empty vial as an alcoholic solution and dried to afford a film. The vial is next flushed with nitrogen and closed until further use. The kit contains only one vial with a predetermined cut off based on one Dornic level, or contains several vials at different Dornic acidity detection limits. In the utilization of this device, a known amount of milk is added via a delivery system to the vial and the vial is shaken for about 20 s. The Dornic acidity of the milk is determined by the lack of or presence of the pink color. If the solution turns colorless, the Dornic acidity of the breast milk is too high, and the mother should dispose of her milk.

### Example 6

### Spoilage Detection

Milk at different freshnesses ranging from 1 to 15° Dornic acidity were added into a vial in addition to a control of rehydrated infant formula (3 mL). A 0.5 mL aliquot of a tetrazolium salt mixture was added to the vials. The vials were then closed with a stopper shaken briefly and let stand for 20 minutes. The vials containing fresh milk with little bacteria content showed a brown color as did the infant formula control. When the bacteria content of the milk is high the solution retains a yellow coloration. This change in color (going from yellow to brown) indicates that the milk remains fresh (See FIG. 26)

### Example 7

### Spoilage Detection

To keep the sodium hydroxide solution separate from the phenolphthalein, the kit can be prepared as follows. The sodium hydroxide solution is introduced into the bottom of a translucent empty vial as an alcoholic solution and dried to afford a film. The phenolphthalein solution is introduced into the cap of a translucent empty vial as an alcoholic solution and dried to afford a film. The vial is next flushed with nitrogen and closed until further use. The kit contains only one vial with a predetermined cut off, or contains several vials at different Dornic acidity detection limits In the utilization of this device, a known amount of milk is added via a delivery system to the vial, the vial is closed, and the vial is shaken for about 20 s. The amount of Dornic acidity content is determined by the lack of or presence of the pink color. If the solution turns colorless, the Dornic acidity of the breast milk is too high, and the mother should dispose of her milk.

### Example 8

### Spoilage Detection

A kit can be prepared as follows. The sodium hydroxide/phenolphthalein solution is introduced into several translucent empty vials (e.g., two vials) as alcoholic solution(s) and dried to afford a film. The vials are next flushed with nitrogen and closed until further use. The amount of base added to each vial is slightly different, such that a scale is created wherein one or more of the vials will turn colorless. This can be done to more accurately determine the Dornic acidity. A known amount of milk is added via a delivery system to the vial, the vial closed, and the vial is shaken for about 20 s. The Dornic acidity of milk is determined by the lack or presence of the pink color. If the solution turns colorless, the Dornic acidity of the breast milk is too high, and the mother should dispose of her milk.

### Example 9

### Spoilage Detection

To keep the sodium hydroxide solution separate from the phenolphthalein, the kit can be prepared as follows. The sodium hydroxide solution is introduced into the bottom of translucent empty vials as an alcoholic solution and dried to afford a film. The phenolphthalein solution is introduced into the caps of translucent empty vials as an alcoholic solution and dried to afford a film. The vials are next flushed with nitrogen and closed until further use. The amount of base added to each vial is slightly different, such that a scale is created wherein one or more of the vials will turn colorless. This can be done to more accurately determine the Dornic acidity. A known amount of milk is added via a delivery system to the vial and the vial shaken for about 20 s. The Dornic acidity of milk is determined by the lack or presence of the pink color. If the solution turns colorless, the Dornic acidity of the breast milk is too high, and the mother should dispose of her milk.

### Example 10

### Spoilage Detection

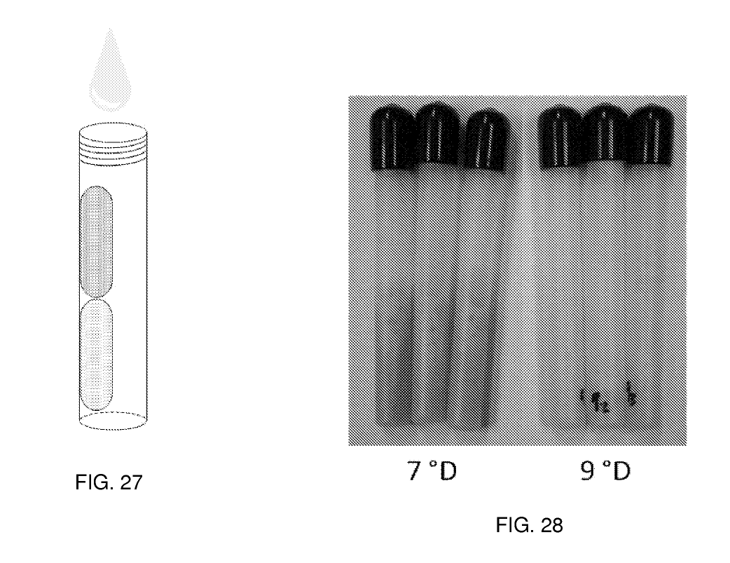

To keep the sodium hydroxide solution separate from the phenolphthalein, the kit can be prepared as follows. The sodium hydroxide solution is introduced into a crushable glass ampoule. The phenolphthalein solution is introduced into a second crushable glass ampoule as an alcoholic solution or as powder. The crushable vials are next flushed with nitrogen or not, sealed, and inserted into a bigger soft plastic vial (FIG. 27). The kit contains only one sodium hydroxide crushable vial with a predetermined cut off, or contains several crushable vials at different Dornic acidity detection limits. This can be done to more accurately determine the Dornic acidity. A known amount of milk is added via a delivery system to the soft plastic vial, the vial is closed, and the soft plastic vial is squeezed crushing the breakable vials contained therein. The plastic vial is shaken for about 20 s. The Dornic acidity of milk is determined by the lack or presence of the pink color. If the solution turns colorless, the Dornic acidity of the breast milk is too high, and the mother should dispose of her milk.

### Example 11

### Spoilage Detection

A kit can be prepared as follows. The sodium hydroxide solution is added to a section of pH paper. Next, 20 microliters of breast milk are added to the pH strip and the color changes. If the color remains purple, then the Dornic acidity of the breast milk is equal to or above 8°. If the color is green or changes from purple to green, the Dornic acidity is too high, and the mother should consider disposing her milk.

### Example 12

### Spoilage Monitor

The overall size of the device is rather small needing to be about 8 cm high and 1 cm in diameter. The usage procedure will follow the following steps: 1) 1 mL of milk is placed into the vial using the supplied syringe; 2) the cap is screwed onto the vial sealing the chamber; 3) the user squeezes the tube at two locations to break the ampoules and shakes to mix; 4) the device is set aside for a predetermined amount of time to ensure final coloration is reached; 5) If the milk turns a color such as pink, the milk is spoiled. 6) the sealed container is disposed of without reopening the cap.

### Example 13

### Accuracy of Calorie Monitor Using Drop Rates/Speeds

A prototype tester for spoilage detection of the type listed above was created containing two glass ampoules: 1) containing sodium hydroxide calibrated to a final Dornic acidity of 8° D; and 2) containing a dye solution. The overall device is 8 cm tall and 0.85 cm in diameter. 1 mL samples of 4× diluted formula adjusted to 7° D and others adjusted to 9° D were added in triplicate to the prototype indicators. The caps were closed, both ampoules were crushed, and the prototypes were shaken to mix the fluids. As expected, the color on the 7° D solutions changed pink, while those testing the 9° D samples remained green suggesting that they would be unsafe to drink (FIG. 28).

### Example 14

### Spoilage Prototype Stability

The spoilage prototype was subjected to an accelerated stability study. The device was incubated at 50° C. with a relative humidity of 50% for 14 weeks. The performance was monitored by removing the tester and monitoring the developed color compared against the coloration of a prototype kept at room temperature. In this model, 1 week under these conditions is equivalent to 8 weeks of RT storage. No degradation in the characteristics of both the dye or base has been observed equating to slightly over 2 years of room temperature storage.

### Example 15

### Endotoxin Detection in Water

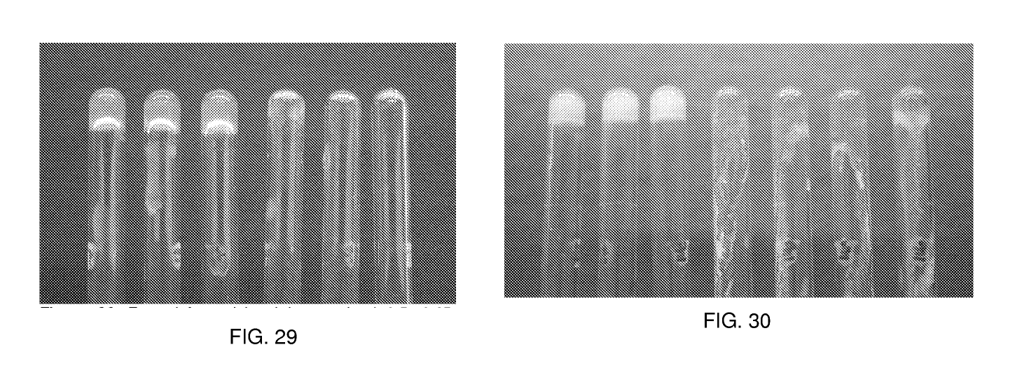

To examine the validity of the LAL test we began with sterile water samples doped with E. coli O55:B5 endotoxin (Lonza; Walkersville, Md.) which was reconstituted to a final concentration of 20 EU/mL. Limulus Amebocyte Lysate (LAL; Lonza) was dissolved in endotoxin free water to a final concentration of 42.9 mg/mL. This LAL amount was chosen so that the gel cutoff was achieved by an endotoxin level of 0.125 EU/mL. Samples of the same endotoxin free water were then doped with the E. coli endotoxin to achieve a logarithmically spaced concentration series of 0.5, 0.25, 0.125, 0.0625, 0.0312, and 0 EU/mL. The samples were mixed in an endotoxin free vial by combining 100 μL of the endotoxin samples and 100 μL LAL solution and incubated for 37° C. in a heating block for 60 minutes. After the incubation, the vials were inverted and photographed to determine whether the gel retained sufficient compositional strength to resist the gravitational forces. As can be seen in the photos the system functioned as expected where the high concentrations of 0.5, 0.25, and 0.125 were gelled while the lower concentrations of endotoxin free control flowed (FIG. 29).

### Example 16

### Endotoxin Detection in Human Milk

After validating the proof-of-principle with water, we next examined whether detecting endotoxins in breast milk was feasible. Such a test has not been previously reported in human breast milk. As can be imagined, breast milk presents a complex environment in which to run this assay with a wide variety of salts, proteins, fats, and carbohydrates that could interfere with the gel formation.⁹² Additionally, LAL is sensitive to pH and must be run at pHs between 6.0 and 8.0 (per the manufacturer product manual). Fortunately, breast milk, though it does vary slightly in pH, is physiologically confined between a pH of 7.1 and 7.4 for all mothers negating this concern.⁹³ We began by testing the LAL method outlined above with 1 day old breast milk that had been refrigerated after collection. The milk was doped with varied levels of E. coli endotoxin to achieve final exogenous concentrations of 0.5, 0.25, 0.125, 0.0625, 0.0312, and 0 EU/mL of milk in addition to an additional control of undoped milk that would not have any LAL added to it to determine if the 37° C. heating process had any discernable effects. Next, 100 μL of the doped and undoped milk samples were added to a glass endotoxin free vial and were mixed with 100 μL of the LAL solution before incubation in a heating block for 1 hour at 37° C. After 1 hour, the tubes were inverted and photographed to examine for the presence of a formed gel (FIG. 30). As can clearly be seen in the image, the test kit performed identically to its performance in water gelling at concentrations of 0.125 EU/mL and greater as designed. The gel is easy to see using the naked eye and so presents a rapid and convenient method to examine for the presence of endotoxins.

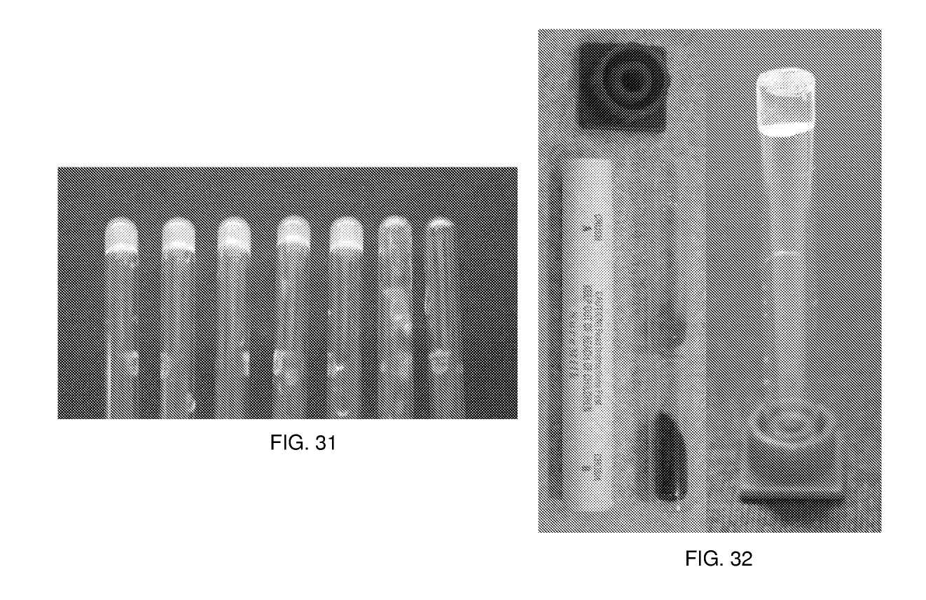

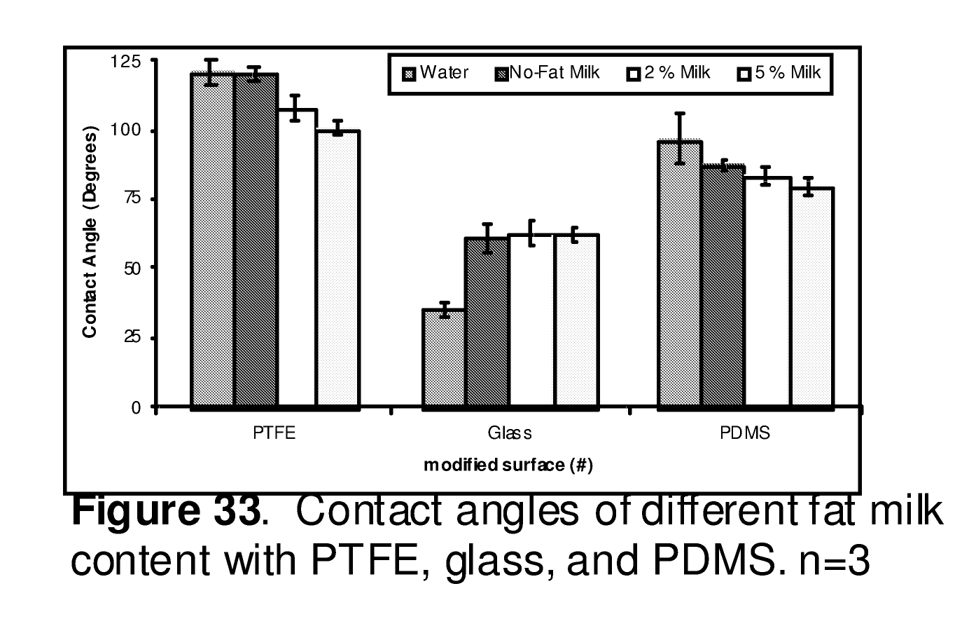

A second consideration of the kit would be in examining how stored milk would function in regards to gel formation. A sample of milk was collected and immediately frozen at −20° C. for 8 months. Upon thawing the milk was examined and found to form fat globules, which is not uncommon for prolonged milk storage.⁹⁴ To remove these globules the milk was briefly sieved through a filter. Next, the milk was again doped with the same exogenous endotoxin using identical concentrations as outlined above and the experiment was repeated by mixing the milk with the LAL reagent and incubating for 60 minutes. A water control was run in parallel to verify that the reagents functioned as expected. The inverted vials were photographed for the presence of the gel (FIG. 31). As can be observed, a shift occurred at what amount of doped endotoxin formed a gel. All doped endotoxin concentrations formed a gel, while the sample with 0 EU/mL did not. Clearly this sample did not endogenously possess sufficient endotoxin, however, a small exogenous addition of 0.0312 EU/mL was enough to tilt the balance. Therefore, we can conclude that this particular sample had an endogenous endotoxin concentration less than 0.125 but more than 0.0625 EU/mL. These experiments show the versatility of such a test, but again, the produced product will be designed with an inherent cutoff selected in line with the needs of the hospitals and milk banks. If a binary test were run on this sample with the cutoff being 0.125 EU/mL than this sample would have not gelled and would pass screening in regards to its endotoxin concentration. This prolonged storage for 8 months involved immediate freezing in dry ice after pumping and then careful temperature control at −20° C., a process that would be extremely difficult for mothers to replicate in their commercial freezers and so their samples will more readily promote endotoxin formation as has been noted in the literature.¹⁰ Milk donated to the banks kept at home for similar amounts of time would most likely not pass the screening.

### Example 17

### Initial Endotoxin Detector Prototype Design

The requirements for the device design are fourfold: 1) the reagents must not come into contact with the milk until the device is closed; 2) the method of delivering the milk and reagents into the sample must be straightforward and accurately controlled; 3) the entire device must be easy to operate; and 4) the results must be easy to read. To meet these requirements we have created a prototype comprised of a flexible vial with a cap holding two crushable glass ampoules. One ampoule will contain the LAL and the other, if needed, will contain a dye that will allow for easier visualization of the gel (FIG. 32). The vial will be wrapped in a removable paper sleeve to ensure that upon squeezing the closed vial to break the ampoules, no injuries to the user's fingers occur. We have tested crushing the ampoules over a hundred times and the glass puncturing the vial has yet to occur. Additionally, the cap used will contain a base upon which the inverted tube can be set for viewing. The user will be supplied with a sterilized syringe to accurately measure 100 μL of milk. The overall size of the device is rather small needing to be only 8.5 cm high and ¾ cm in diameter. The usage procedure will be: 1) 100 μL of milk is placed into the vial using the supplied syringe; 2) the cap is placed onto the vial sealing the chamber; 3) the user squeezes the tube at two locations to break the ampoules containing the LAL and dye, if needed, and shakes to mix; 4) the sleeve is removed and the tube is placed into a 37° C. heating block or water bath and left for 1 hour; 5) after 1 hour the device is retrieved, inverted, and set upright on the cap; 6) the user determines if a gel was formed and thereby determines the endotoxin safety of the milk sample either disposing of the original milk container or setting it aside for pasteurization; and 7) the sealed container is disposed of without reopening the cap. If an elevated level of endotoxin is reported, the user will be advised to retest to ensure the result was not a false positive. Additionally, positive controls will be supplied with the kit with ampoules containing the LAL and a sample of lyophilized endotoxin adjusted to just above the cutoff point. This control test can be run to ensure that the LAL is functioning correctly and would rule out the occurrence of any false positives or false negatives.

### Example 18

### Contact Angle of Milk on Surfaces

The caloric monitor is based on the principles of surface tension forces and surface free energy. We are using these principles to detect the changes in breast milk fat content with our monitor. Specifically, the monitor relies upon the change in hydrophobicity of the breast milk sample, which is directly related to the fat concentration. Breast milk containing 2% vs. 10% w/v fat will interact differently with a surface. The type of surface (more or less hydrophobic) and the size and shape of a drop of a liquid can affect the interaction between the surface and the drop. For example, a drop of water will minimize its contact with a hydrophobic surface by increasing the contact angle. In our first experiment, we measured the contact angle of no-fat, 2%, and 5% milk on three common surfaces—PTFE (polytetrafluoroethylene), glass, and PDMS (polydimethylsiloxane). As shown in FIG. 33, a trend can be observed between the fat content and contact angle on the two hydrophobic surfaces, however, on the glass surface no such dependence was observed.

### Example 19

### Rate of Milk Transport Down a Hydrophobic Surface

Another embodiment of the importance of surface energies involves the rate of transport of varied fat content milks down an angled surface. Equal volumes of water and no-fat, 2% and whole milk (100 μL) were placed on a flat PTFE surface. The slope of the surface was increased gradually until all droplets had rolled down the incline. As expected the more hydrophobic droplets had less resistance to interacting with the PTFE surface and thus began to move at a lower incline angle. The droplets moved in order with the whole milk and 2% milk releasing before the no-fat milk and water which both moved at a high incline at relatively the same time. A correlation between milk fat and resistance to motion on a hydrophobic incline is therefore readily observed.

### Example 20

### Rate of Milk Passage Through a PTFE Tube

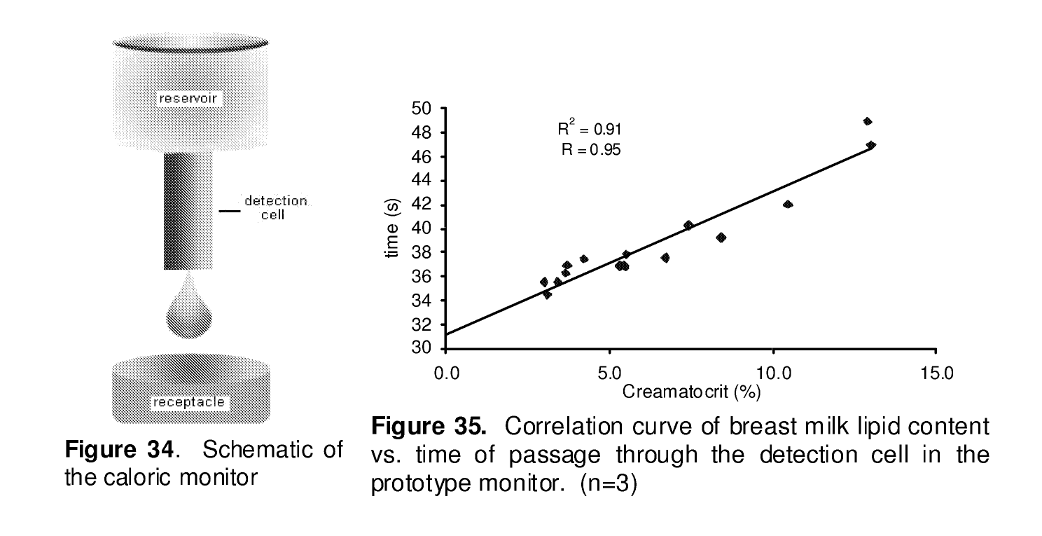

The basic design of the monitor is shown in FIG. 34. The monitor consists of a reservoir for holding the breast milk sample, a detection cell that has a specific surface for interacting with the breast milk sample, and a receptacle for collecting the breast milk. As the milk passes through the detection cell, which is composed of the modified polystyrene, its rate of passage is dependent on its fat content. As such we can measure the time necessary for breast milk to flow through the detection cell and can correlate this to a specific fat content of the breast milk. Using this technology, we can quickly measure the fat/caloric content of breast milk using small volume samples (<1 mL).

### Example 21

### Accuracy of Calorie Monitor

The accuracy of the technology was determined using breast milk samples from four voluntary donors (with multiple samples from each donor). The fat concentration of the breast milk was obtained using Creamatocrit Plus™ (Medela). For our monitor, we measured the average time for 40 drops to pass through the detection cell. A correlation of r=0.95 was obtained using this laboratory prototype monitor (FIG. 35). The correlation between our device and the fat concentration is good, even though we are limited by the error on the x-axis generated by measurements taken from the Creamatocrit plus™ and by the error on the y-axis generated by the laboratory prototype which uses manual timing of the drop speeds. Currently, we are using a human-operated timer and a hand-made detection cell which generates a slight variability on each data point (±0.5 s). Using a simple electronic counter instead of a manual counter to determine the time necessary for the drop to flow through the detection cell we can improve this measurement.

### Example 22

### Use of the Calorie Monitor to Alter Eating Habits and Thus the Caloric Content of Breast Milk

A plot of the calorie content as a function of time and food intake is obtained, which enables a mother to identify the best time to feed her newborn to ensure an adequate amount or even a high amount of fat or calorie content in her breast milk. By doing so, newborns can receive the calories that are needed for proper development. This device and kit is especially useful for mothers in the feeding of infants and newborns who are of low birth-weight or are not gaining sufficient weight as a function of time. To aid in this endeavor each kit will contain a logbook and/or chart and/or website address where the mother may record her caloric history. This will enable mothers to keep track of such important variables as the historical readings, the time of day, time since last meal, and meal portion and type. In this manner information can be retrieved allowing the mother to make informed decision on when is the best time to breastfeed to obtain optimal caloric nutrition for the infant. The logbook, chart, or web database will allow the mother to privately maintain this important information.

### Example 23

### Enzymatic Detection of Hg

We selected urease as the enzyme for this monitor for the following four reasons. First, urease has been shown to be sensitive to Hg and insensitive to other heavy metal ions such as cadmium, lead, zinc, and nickel.¹⁰⁹ Secondly, mercury can affect the activity of the enzyme at concentrations down to 1 ppb. Thirdly, urease catalyzes the conversion of urea into carbon dioxide and ammonia [(NH₂)₂CO+H₂O→CO₂+2NH₃] and thus in aqueous solution increases the pH. Fourthly, urea is a stable enzyme that is not denatured until a temperature of 72° C. is reached and is readily stored lyophilized for two years at 4° C., indicative of good shipment and storage characteristics.¹¹⁰ Milk that contains differing amounts of mercury results in different final colorations. As described, we have shown that a monitor based on these principles can be created for mercury concentration detection in solutions as diverse as water and infant formula. We have also shown that the color change in formula is sensitive to parts-per-billion of mercury alone and not competing ions of iron, copper, manganese, zinc, and various other metallic ions which are all present in infant formula in concentrations a thousand times greater than mercury (diluted formula produces similar results).

### Example 24

### Detecting Mercury in Water

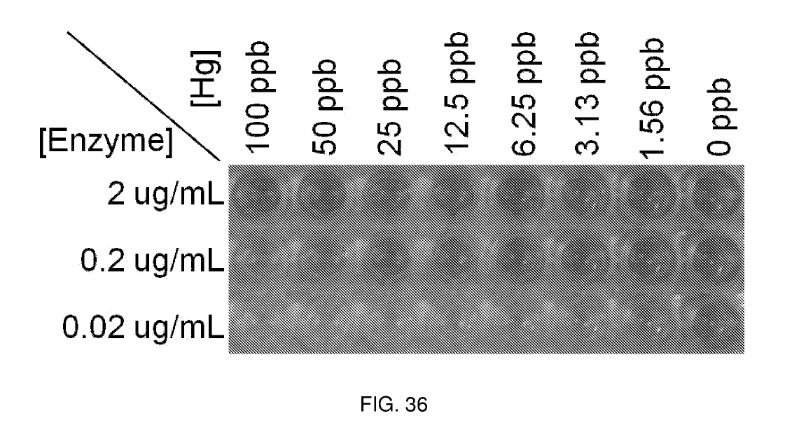

Urease is an active enzyme with a high activity unit per mg of powder. Consequently, a very small amount of enzyme is capable of catalyzing the conversion of a large amount of urea into ammonium ions. A wide variety of pH dyes and mixtures of dyes that met these requirements were tested such as 3-nitrophenol, phenol red, neutral red, phenolphthalein, thymol blue, and cresol red to name a few. A dye combination that produced optimal results in infant formula was found that is colored yellow at neutral pH but transitions to green and finally a blue/indigo coloration upon enzyme activity. Because formula is a buffered solution, as is breast milk, we elected to begin testing of the method using a 96-well plate format on non-buffered water. Mercury(II) trifluoroacetate (Sigma) was dissolved in nanopure water (17.9 MΩ-cm) to produce a stock solution of 2000 ppb (μg Hg/L) mercury ions. The stock concentration of mercury was diluted to produce a physiological logarithmic range of values from 1.56 to 100 ppb (7 points total). Additionally, control values of 0 and 1000 ppb were also included. Various concentrations of enzyme (Urease Type III from Jack Bean, Sigma) were added to the wells along with the dye mixture and the plates were let stand for 10 minutes at room temperature (RT). Urea (Sigma) was added in excess so that it would not affect the kinetics of the experiment and the color change was recorded over time with a camera (Canon EOS Digital Rebel) as well as monitored by watching the color change over time. The reaction proceeded as expected with higher amounts of mercury and lower enzyme concentrations taking longer to produce a color change (FIG. 36). Wells with no mercury changed the fastest as there was no enzyme inactivation and control wells that contained all components except for the enzyme or the substrate never changed color proving that the change was not due to a specific component alone but required the combination of mercury, dye, urease, and urea.

### Example 25

### Detecting Mercury in Infant Formula

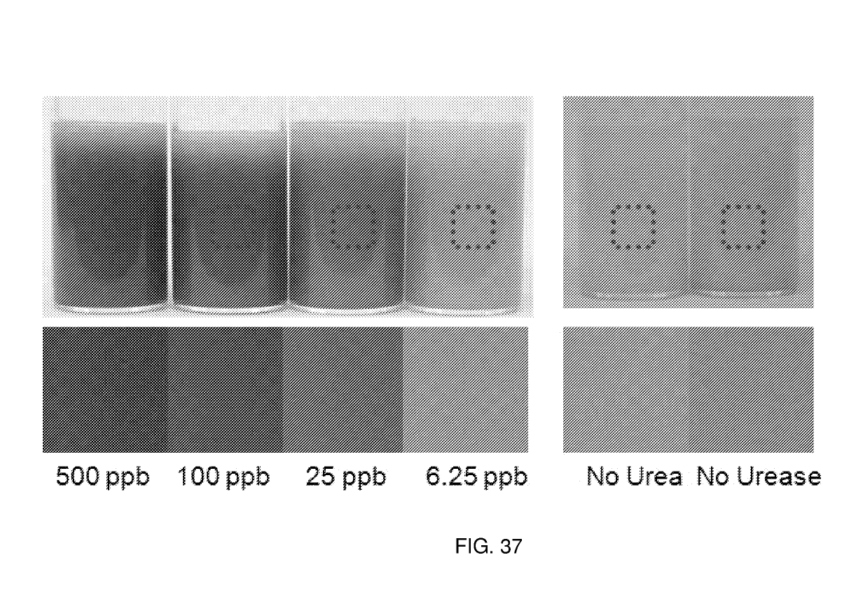

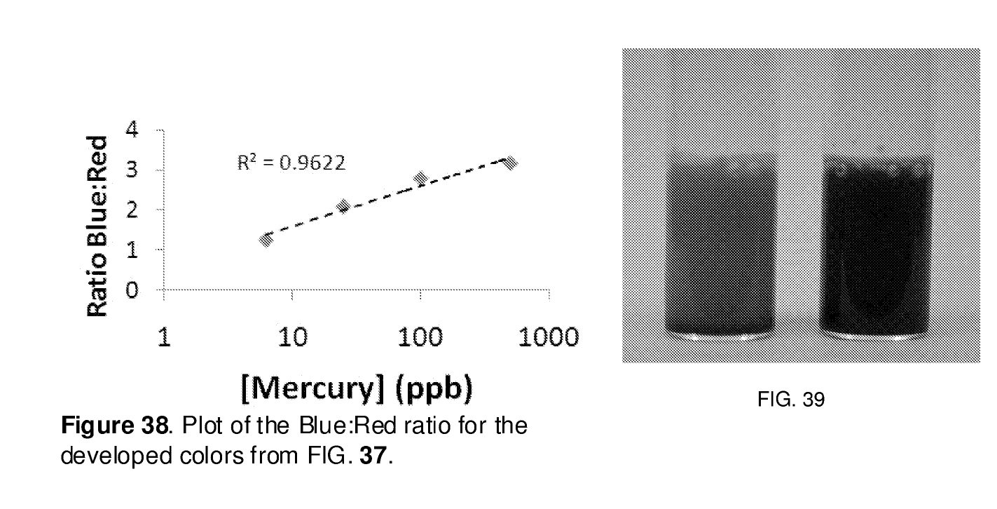

Prior to working with breast milk we have performed studies with infant formula because it is consistent between doses, more readily available, and contains a minimal amount of endogenous mercury (with breast milk this amount would be unknown). Formula presents a more complicated environment in which to test the assay than what is provided in water. Infant formula is not only buffered, but contains a myriad of proteins with which the metal ions could also interact. Infant formula (Nestlé) was mixed with nanopure water according to manufacturer directions. This was followed by the addition of varied amounts of mercury, the dye solution, urease, and finally urea producing a final volume of 3 mL. After overnight room temperature reaction to ensure complete color development, the results showed a consistent color gradient that depended on the amount of mercury present (FIG. 37). Again, controls without either enzyme or substrate did not produce a color change and these controls remained the neutral yellow color for the course of the experiment (FIG. 37). Taking the ratio of the blue:red coloration in the pixels contained in the red dashed boxes in the figure (and blown up immediately below), there is a clear linear trend dependent on Hg concentration (R²=0.96; FIG. 38). This verifies the use of the enzymatic reaction to calculate the amount of mercury at physiological concentrations.

Formula contains a variety of metals at concentrations many times above that of the mercury (Table 3) and the recorded color gradient was insensitive to these ions, diluted formula samples containing less of these ions still produced a color gradient. Final colorations are currently achieved after a few hours in solution, but by varying the relative amounts of dye/urease/urea we have shown in both water and formula that quicker or slower timings can easily be achieved.

### Example 26

### Detecting Hg in Breast Milk

Milk taken from a donor mother was doped with either a high concentration of mercury. An undoped milk was used as a control. Urease enzyme was added in addition to the bromothymol blue mixture and let stand for 5 minutes. The urea substrate was then added and imaged over time. An identical yellowish-green coloration was initially observed in both conditions with the no mercury sample eventually turning a dark green and then a dark blue color while the Hg-doped sample coloration remained unchanged. After an hour and a half the results were especially pronounced and are shown in FIG. 39. A correlation between sample color and mercury concentration is readily observed in a human breast milk sample.

### Example 27

### Hg Monitor

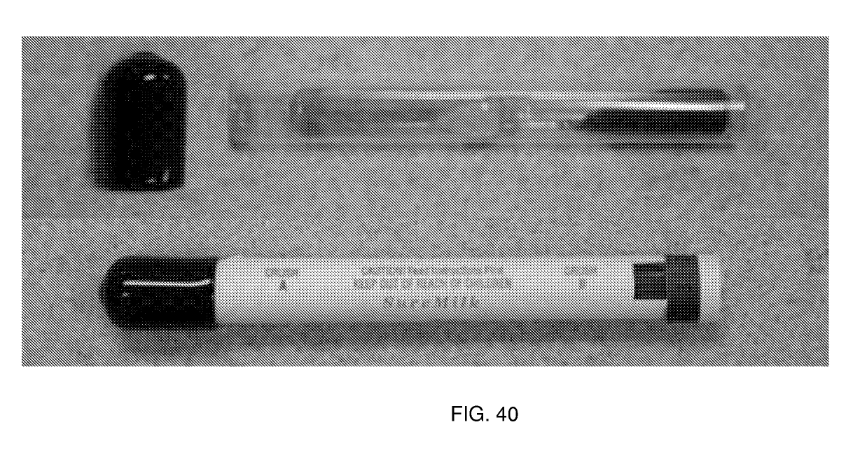

The requirements for the device design are fourfold: 1) the reagents must not come into contact with the milk until the device is closed; 2) the method of delivering the milk and reagents into the sample must be straightforward and accurately controlled; 3) the entire device must be easy to operate; and 4) the results must be easy to read. To meet these requirements we have created a prototype comprised of a flexible vial with a cap holding two crushable glass ampoules. One ampoule will contain the enzyme and the other will contain the dye and substrate (FIG. 40). The entire vial will be wrapped in a removable paper sleeve to ensure that upon squeezing the closed vial to break the ampoules, no injuries to the user's fingers occur. We have yet to have a piece of glass puncture the vial after testing the system over a hundred times. Additionally, this paper sleeve will have a small opening through which the final color can be read and the litmus-type scale will be printed below this window to allow the user to match the developed color with the closest match on the gradient scale. An outcomes table will be supplied with the product (please see c.5.) which will convert the color to the US ASTDR mercury recommendation for infants of a particular weight. Although milk banks will most likely use a system where a certain ppb Hg in the milk will be discarded as they do not know ahead of time where the milk will be sent and hence what weight the infant will be. There will be 7 colors in the gradient scale that will represent values over the relevant physiological mercury concentrations (0-25 ppb) and each color will have a specific number assigned to it for ease of use and recall. The user will be supplied with a sterilized syringe to accurately measure 1 mL of milk. The overall size of the device is rather small needing to be only 8.5 cm high and ¾ cm in diameter. The usage procedure will be: 1) 1 mL of milk is placed into the vial using the supplied syringe; 2) the cap is placed onto the vial sealing the chamber; 3) the user squeezes the tube at two locations to break the ampoules and shakes to mix; 4) the device is set aside for a predetermined amount of time to ensure final coloration is reached; 5) rotating the sleeve to move the window the user finds the gradient color that best matches the developed color; 6) the user consults the supplied outcome table to determine the safety of their milk in regards to the governmental recommendation;⁵⁷ and 7) the sealed container is disposed of without reopening the cap.

### Example 28

### Reading the Results of the Hg Monitor

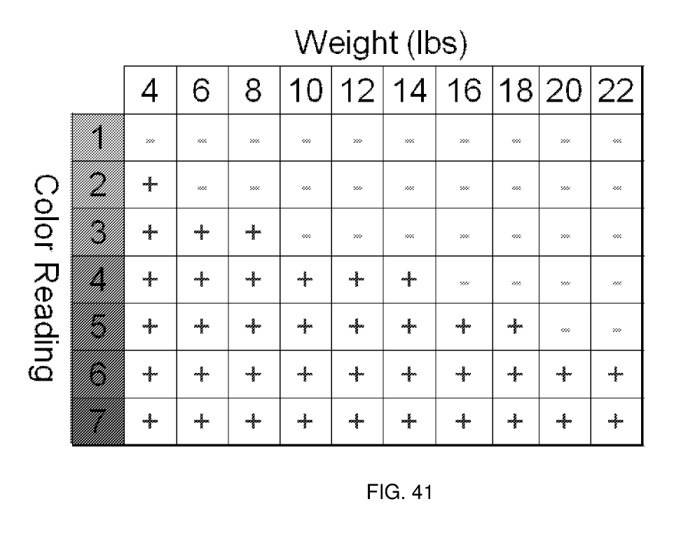

An outcomes table will be supplied with the product which will convert the color to the US ASTDR mercury recommendation for infants of a particular weight. There will be 7 colors in the gradient scale that will represent values over the relevant physiological mercury concentrations (0-25 ppb) and each color will have a specific number assigned to it for ease of use and recall. Because the weight of the infant is important in determining the tolerable mercury intake, the outcome regarding breast milk concentrations that are “safe” and those that the mother should talk to her health care professional will vary depending on the child's size. To solve this problem, each kit will contain a sliding chart that the mother will adjust so that the weight of her child is visible as the selectable criteria. Next, she/he will take the color reading from the device and use this to index the recommendation on the table regarding her mercury concentration (FIG. 41). As an example, a breast milk concentration of 5 μg Hg/kg of milk is considered “safe” for children over 5.5 pounds, but not those under and the chart will reflect this reality. The gradient scale will be set according to the color values obtained and adjusted in Aim 1 below so as to provide the maximum amount of information to the mother. Those mothers who have initial mercury readings above the recommended level for their child will be encouraged to repeat the readings to ensure that the initial measurement was not a false positive and then to consult their health care provider.

### Example 29

### Accuracy of Calorie Monitor Using Drop Counts/Volumes

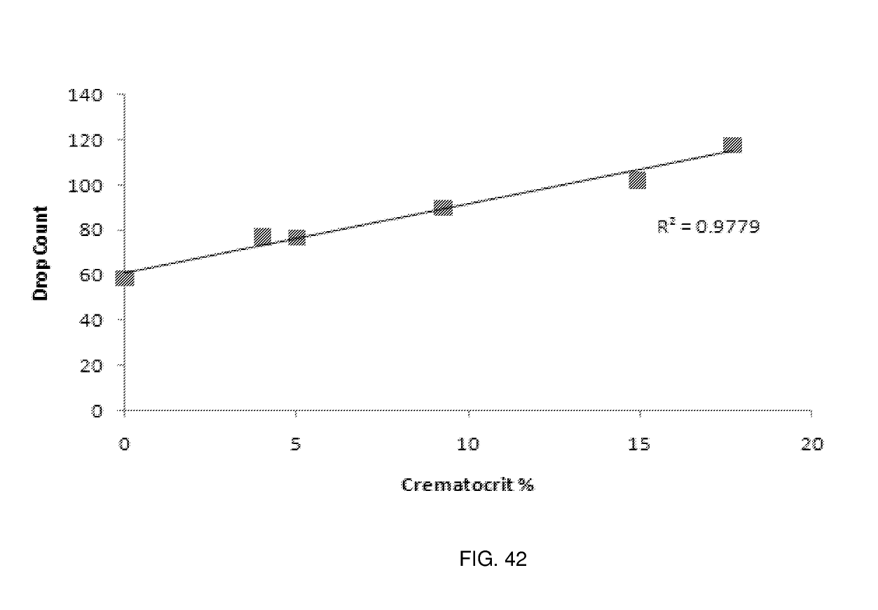

The accuracy of the technology was also determined using breast milk samples from additional donors, water, and formula (6 total samples, 3 trials per sample). The fat concentration of the breast milk was obtained using Creamatocrit Plus™ (Medela). For our monitor, we measured total drop count produced from a 1 mL sample of milk. The size of the drop is controlled by the rate of flow through the detection cell and/or the surface energy relationship between the forming droplet and the geometry and material of the tube ending. The size of the resulting drop is controlled by when the pull of gravity and the momentum of the liquid exiting the tube, overcomes the surface tension and surface energy interaction of the liquid/tube material. A correlation of r=0.989 was obtained using this laboratory prototype monitor (FIG. 42). The correlation between our device and the fat concentration is good, even though we are limited by the error on the x-axis generated by measurements taken from the Creamatocrit plus™. Because a set volume of milk is used as the input to each measurement and the total drop count is measured, the volume per drop can be calculated indirectly by taking the input volume 1 mL, and dividing it by the total number of drops counted. High correlations between the droplet volume and fat/calorie concentrations are observed with the indirect method or a direct weighing method are used.

### INCORPORATION BY REFERENCE

All of the U.S. patents and U.S. patent application publications cited herein are hereby incorporated herein by reference.

### Equivalents

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### REFERENCES CITED

(1) Beaudry, M.; Dufour, R.; Marcoux, S. J Pediatr 1995, 126, 191-7.

(2) Dewey, K. G.; Heinig, M. J.; Nommsen-Rivers, L. A. J Pediatr 1995, 126, 696-702.

(3) Howie, P. W.; Forsyth, J. S.; Ogston, S. A.; Clark, A.; Florey, C. D. Bmj 1990, 300, 11-6.

(4) Wilson, A. C.; Forsyth, J. S.; Greene, S. A.; Irvine, L.; Hau, C.; Howie, P. W. Bmj 1998, 316, 21-5.

(5) Wright, A. L.; Holberg, C. J.; Martinez, F. D.; Morgan, W. J.; Taussig, L. M. Bmj 1989, 299, 946-9.

(6) Pisacane, A.; Graziano, L.; Mazzarella, G.; Scarpellino, B.; Zona, G. J Pediatr 1992, 120, 87-9.

(7) Aniansson, G.; Alm, B.; Andersson, B.; Hakansson, A.; Larsson, P.; Nylen, O.; Peterson, H.; Rigner, P.; Svanborg, M.; Sabharwal, H.; et al. Pediatr Infect Dis J 1994, 13, 183-8.

(8) Duncan, B.; Ey, J.; Holberg, C. J.; Wright, A. L.; Martinez, F. D.; Taussig, L. M. Pediatrics 1993, 91, 867-72.

(9) Lucas, A.; Cole, T. J. Lancet 1990, 336, 1519-23.

(10) Ogundele, M. O. Eur J Pediatr 2000, 159, 793-7.

(11) Scheifele, D. W.; Olsen, E.; Fussell, S.; Pendray, M. J Pediatr Gastroenterol Nutr 1985, 4, 67-74.

(12) Scheifele, D. W.; Olsen, E. M.; Pendray, M. R. Am J Clin Pathol 1985, 83, 227-9.

(13) Townsend, S.; Caubilla Barron, J.; Loc-Carrillo, C.; Forsythe, S. Food Microbiol 2007, 24, 67-74.

(14) Hurley, J. Br J Surg 1993, 80, 809.

(15) Hurley, J. C. Clin Microbiol Rev 1995, 8, 268-92.

(16) Morrison, D. C.; Ryan, J. L. Annu Rev Med 1987, 38, 417-32.

(17) Fumarola, D.; Miragliotta, G.; Monno, R. Eur J Epidemiol 1987, 3, 454-5.

(18) Oppenheim, B. A.; Barclay, G. R.; Morris, J.; Knox, F.; Barson, A.; Drucker, D. B.; Crawley, B. A.; Morris, J. A. Arch Dis Child 1994, 70, 95-8.

(19) FDA, Ed. March 1985.

(20) Nakata, T. J Pharm Sci Technol 1994, 48, 59-63.

(21) Ludwig, J. D.; Avis, K. E. J Parenter Sci Technol 1990, 44, 4-12.

(22) Tsuji, K.; Harrison, S. J. Appl Environ Microbiol 1978, 36, 710-4.

(23) Daneshian, M.; Guenther, A.; Wendel, A.; Hartung, T.; von Aulock, S. J Immunol Methods 2006, 313, 169-75.

(24) Moore, K. J.; Andersson, L. P.; Ingalls, R. R.; Monks, B. G.; Li, R.; Arnaout, M. A.; Golenbock, D. T.; Freeman, M. W. J Immunol 2000, 165, 4272-80.

(25) Wilson-Clay, B. Int Breastfeed J 2006, 1, 6.

(26) Bang, F. B. Bulletin of the Johns Hopkins Hospital 1956, 98, 325-351.

(27) Levin, J.; Bang, F. B. Thrombosis et diathesis haemorrhagica 1968, 19, 186-197.

(28) Solum, N. O. Thrombosis et diathesis haemorrhagica 1970, 23, 170-181.

(29) Young, N. S.; Levin, J.; Prendergast, R. A. J Clin Invest 1972, 51, 1790-7.

(30) U.S. Department of Health and Human Services, P. H. S., Food and Drug Administration 1987.

(31) Guidelines for the Establishment and Operation of a Donor Human Milk Bank; 9 ed.; Human Milk Banking Association of North America: Raleigh, N.C., 2000.

(32) Arnold, L. D. J Hum Lact 1999, 15, 55-9.

(33) Gutierrez, D.; de Almeida, J. A. J Hum Lact 1998, 14, 333-5.

(34) Springer, S. J Hum Lact 1997, 13, 65-8.

(35) Dewey, K. G.; Lovelady, C. A.; Nommsen-Rivers, L. A.; McCrory, M. A.; Lonnerdal, B. N Engl J Med 1994, 330, 449-53.

(36) Ford, R. P.; Taylor, B. J.; Mitchell, E. A.; Enright, S. A.; Stewart, A. W.; Becroft, D. M.; Scragg, R.; Hassall, I. B.; Barry, D. M.; Allen, E. M.; et al. Int J Epidemiol 1993, 22, 885-90.

(37) Rigas, A.; Rigas, B.; Glassman, M.; Yen, Y. Y.; Lan, S. J.; Petridou, E.; Hsieh, C. C.; Trichopoulos, D. Ann Epidemiol 1993, 3, 387-92.

(38) Wang, Y. S.; Wu, S. Y. J Hum Lact 1996, 12, 27-30.

(39) Newcomb, P. A.; Storer, B. E.; Longnecker, M. P.; Mittendorf, R.; Greenberg, E. R.; Clapp, R. W.; Burke, K. P.; Willett, W. C.; MacMahon, B. N Engl J Med 1994, 330, 81-7.

(40) Rosenblatt, K. A.; Thomas, D. B. Int J Epidemiol 1993, 22, 192-7.

(41) Cumming, R. G.; Klineberg, R. J. Int J Epidemiol 1993, 22, 684-91.

(42) Melton, L. J., 3rd; Bryant, S. C.; Wahner, H. W.; O'Fallon, W. M.; Malkasian, G. D.; Judd, H. L.; Riggs, B. L. Osteoporos Int 1993, 3, 76-83.

(43) Mayer, E. J.; Hamman, R. F.; Gay, E. C.; Lezotte, D. C.; Savitz, D. A.; Klingensmith, G. J. Diabetes 1988, 37, 1625-32.

(44) Gerstein, H. C. Diabetes Care 1994, 17, 13-9.

(45) Tuttle, C. R.; Dewey, K. G. J Am Diet Assoc 1996, 96, 885-90.

(46) HHS Blueprint for Action on Breastfeeding, U.S. Department of Health and Human Services, 2000.

(47) CDC 2005.

(48) Riordan, J. M. J Hum Lact 1997, 13, 93-7.

(49) Ahluwalia, I. B.; Morrow, B.; Hsia, J. Pediatrics 2005, 116, 1408-12.

(50) Martin, J.; Hamilton, B.; Sutton, P. Births: Final data for 2004. National vital statistics reports, National Center for Health Statistics, 2006.

(51) Meier, P. P.; Engstrom, J. L.; Murtaugh, M. A.; Vasan, U.; Meier, W. A.; Schanler, R. J. J. Perinatol 2002, 22, 646-9.

(52) Lucas, A.; Gibbs, J. A.; Lyster, R. L.; Baum, J. D. Br Med J 1978, 1, 1018-20.

(53) Griffin, T. L.; Meier, P. P.; Bradford, L. P.; Bigger, H. R.; Engstrom, J. L. J Obstet Gynecol Neonatal Nurs 2000, 29, 249-57.

(54) Amin-zaki, L.; Majeed, M. A.; Clarkson, T. W.; Greenwood, M. R. Br Med J 1978, 1, 613-6.

(55) Amin-Zaki, L.; Majeed, M. A.; Greenwood, M. R.; Elhassani, S. B.; Clarkson, T. W.; Doherty, R. A. J Appl Toxicol 1981, 1, 210-4.

(56) Tones, A. D.; Rai, A. N.; Hardiek, M. L. Pediatrics 2000, 105, E34.

(57) ATSDR Toxicological Profile for Mercury, 1999.

(58) CDC Third National Report on Human Exposure to Environmental Chemicals, 2005.

(59) Oken, E.; Wright, R. O.; Kleinman, K. P.; Bellinger, D.; Amarasiriwardena, C. J.; Hu, H.; Rich-Edwards, J. W.; Gillman, M. W. Environ Health Perspect 2005, 113, 1376-1380.

(60) Lauritzen, L.; Hansen, H.; Jorgensen, M.; Michaelsen, K. Prog Lipid Res 2001, 40, 1-94.

(61) Agency, U. S. E. P. Executive Summary, 1997.

(62) Grandjean, P.; Jorgensen, P. J.; Weihe, P. Environ Health Perspect 1994, 102, 74-7.

(63) Young, R. A. Toxicity Summary for METHYL MERCURY, Oak Ridge Reservation Environmental Restoration Program, 1992.

(64) Gundacker, C.; Pietschnig, B.; Wittmann, K. J.; Lischka, A.; Salzer, H.; Hohenauer, L.; Schuster, E. Pediatrics 2002, 110, 873-8.

(65) Hattula, T.; Rahola, T. Environ Physiol Biochem 1975, 5, 252-7.

(66) Vahter, M.; Mottet, N. K.; Friberg, L.; Lind, B.; Shen, D. D.; Burbacher, T. Toxicol Appl Pharmacol 1994, 124, 221-9.

(67) Rahola, T.; Hattula, T.; Korolainen, A.; Miettinen, J. K. Ann Clin Res 1973, 5, 214-9.

(68) Aberg, B.; Ekman, L.; Falk, R.; Greitz, U.; Persson, G.; Snihs, J. O. Arch Environ Health 1969, 19, 478-84.

(69) Miettinen, J. K.; Rahola, T.; Hattula, T.; Rissanen, K.; Tillander, M. Ann Clin Res 1971, 3, 116-22.

(70) Hightower, J.; Moore, D. Environ Health Perspect 2003, 111, 604-608.

(71) Grandjean, P.; Weihe, P.; Needham, L. L.; Burse, V. W.; Patterson, D. G., Jr.; Sampson, E. J.; Jorgensen, P. J.; Vahter, M. Environ Res 1995, 71, 29-38.

(72) Mahaffey, K. R.; Mergler, D. Environ Res 1998, 77, 104-14.

(73) Sanzo, J. M.; Dorronsoro, M.; Amiano, P.; Amurrio, A.; Aguinagalde, F. X.; Azpiri, M. A. Public Health Nutr 2001, 4, 981-8.

(74) Mozaffarian, D.; Rimm, E. B. Jama 2006, 296, 1885-99.

(75) FDA; EPA What You Need to Know About Mercury in Fish and Shellfish: Advice for Women Who Might Become Pregnant, Women Who are Pregnant, Nursing Mothers, and Young Children.

(76) of Geijersstam, E.; Sandborgh-Englund, G.; Jonsson, F.; Ekstrand, J. J Dent Res 2001, 80, 1793-6.

(77) Burbacher, T. M.; Shen, D. D.; Liberato, N.; Grant, K. S.; Cernichiari, E.; Clarkson, T. Environ Health Perspect 2005, 113, 1015-21.

(78) Sherlock, J.; Hislop, J.; Newton, D.; Topping, G.; Whittle, K. Hum Toxicol 1984, 3, 117-31.

(79) Smith, J. C.; Farris, F. F. Toxicol Appl Pharmacol 1996, 137, 245-52.

(80) Golding, J. Early Hum Dev 1997, 49 Suppl, S29-43.

(81) Oskarsson, A.; Schultz, A.; Skerfving, S.; Hallen, I. P.; Ohlin, B.; Lagerkvist, B. J. Arch Environ Health 1996, 51, 234-41.

(82) Bakir, F.; Damluji, S. F.; Amin-Zaki, L.; Murtadha, M.; Khalidi, A.; al-Rawi, N. Y.; Tikriti, S.; Dahahir, H. I.; Clarkson, T. W.; Smith, J. C.; Doherty, R. A. Science 1973, 181, 230-41.

(83) Pitkin, R. M.; Bahns, J. A.; Filer, L. J., Jr.; Reynolds, W. A. Proc Soc Exp Biol Med 1976, 151, 565-7.

(84) Galster, W. A. Environ Health Perspect 1976, 15, 135-40.

(85) WHO; IAEA Minor and Trace Elements in Breast Milk, 1989.

(86) Dye, J. L.; Bureau, U. C., Ed. 2004.

(87) League, L. L. 2006.

(88) Administration, U. S. F. a. D. 2007.

(89) Pediatrics, A. A. o.

(90) Duke, C. S. New Beginnings 1998, 15, 109.

(91) MSUcares; Mississipy State University Extension Service: 2003; Vol. 2007.

(92) Goedhart, A. C.; Bindels, J. G. Nutrition Research Reviews 1994, 7, 1-23.

(93) Morriss, F. H., Jr.; Brewer, E. D.; Spedale, S. B.; Riddle, L.; Temple, D. M.; Caprioli, R. M.; West, M. S. Pediatrics 1986, 78, 458-64.

(94) Jensen, R. G. Determinants of milk volume and composition; Academic Press: New York, 1995.

(95) Chaudhury, M. K.; Whitesides, G. M. Science 1992, 256, 1539-1541.

(96) Bertini, I.; Gray, H. B.; Stiefel, E. I.; Valentine, J. S. Biological inorganic chemistry: structure and reactivity; University Science Books: Sausalito, Calif., 2007.

(97) Hughes, M. N. The inorganic chemistry of biological processes; 2d ed.; J. Wiley: Chichester [Eng.]; New York, 1981.

(98) Lippard, S. J.; Berg, J. M. Principles of bioinorganic chemistry; University Science Books: Mill Valley, Calif., 1994.

(99) Eccles, H.; Garnham, G. W.; Lowe, C. R.; Bruce, N. C. Biotechnology Advances 1997, 15, 189.

(100) Fennouh, S.; Casimiri, V.; Geloso-Meyer, A.; Burstein, C. Biosens Bioelectron 1998, 13, 903-9.

(101) Shekhovtsova, T. N.; Chernetskaya, S. V. Analytical Letters 1994, 27, 2883-2898.

(102) Veselova, I. A.; Shekhovtsova, T. N. Analytica Chimica Acta 1999, 392, 151-158.

(103) Amine, A.; Cremisini, C.; Palleschi, G. Mikrochimica Acta 1995, 121, 183-190.

(104) Farina, M.; Brandao, R.; Lara, F. S.; Soares, F. A. A.; Souza, D. O.; Rocha, J. B. T. Toxicology Letters 2003, 139, 55-66.

(105) Sassa, S. Enzyme 1982, 28, 133-45.

(106) Andres, R. T.; Narayanaswamy, R. Analyst 1995, 120, 1549-1554.

(107) Bryce, D. W.; Fernandezromero, J. M.; Decastro, M. D. L. Analytical Letters 1994, 27, 867-878.

(108) Preininger, C. Mikrochimica Acta 1999, 130, 209-214.

(109) Shi, G. Q.; Jiang, G. B. Analytical Sciences 2002, 18, 1215-1219.

(110) Omar, S.; Beauregard, M. Journal of Biotechnology 1995, 39, 221-228.

1. A bottle adapter constructed and arranged for internal and external fixation to a beverage container comprising:
a plug comprising an internal channel and an external portion comprising at least one annular ring;
an external sealing flange connected to the plug and constructed and arranged to be positioned over an exterior portion of the beverage container; and
a dispenser in fluid communication with the internal channel of the plug.

2. The bottle adapter of claim 1, wherein the plug is constructed and arranged to insert into an interior portion of a neck of the beverage container, and the at least one annular ring of the external portion of the plug extends axially from the plug and is constructed and arranged to engage an interior portion of the neck of the beverage container, and wherein the external sealing flange has a resting diameter less than a smallest diameter of the neck of the beverage container.

3. The bottle adapter of claim 2, wherein the dispenser comprises a top portion selected from the group consisting of a nipple top, a sipper-type top, a straw top terminated in a nipple top, a straw top terminated in a sipper-type top, a straw top terminated in a tubular straw opening, a secondary internal tube constructed and arranged to allow for liquid withdrawal from the bottom of the beverage container, and combinations thereof.

4. The bottle adapter of claim 2, wherein the adapter further comprises
a base portion;
a ring clamp;
means for attaching the ring clamp to the base portion; and
a top portion secured by the ring clamp consisting of nipple top, a sipper-type top, a straw top terminated in a nipple top, a straw top terminated in a sipper-type top, a straw top terminated in a tubular straw opening, a secondary internal tube constructed and arranged to allow for liquid withdrawal from the bottom of the beverage container, and combinations thereof.

5. The bottle adapter of claim 2, wherein the adapter is constructed and arranged to interact with a snap-in portion that is constructed and arranged to be inserted into the base portion and secured into place by a closure, wherein the snap-in portion consists of a nipple top, a sipper-type top, a straw top terminated in a nipple top, a straw top terminated in a sipper-type top, a straw top terminated in a tubular straw opening, a secondary internal tube constructed and arranged to allow for liquid withdrawal from the bottom of the beverage container, and combinations thereof.

6. The bottle adapter of claims 2-5, wherein the adapter further comprises a venting mechanism constructed and arranged to allow for communication between an internal portion of the beverage container and an external environment.

7. A method for determining the fat or caloric content of a breast milk sample by exposing the sample to a surface comprising measuring a property of the breast milk sample and surface interaction selected from the group consisting of flowrate, droplet volume, droplet count, droplet timing, droplet contact angle, surface energy relationship between the sample and a surface, and combinations thereof.

8. The method of claim 7, wherein the surface is of a form selected from the group consisting of a channel, groove, tube, and combinations thereof.

9. The method of claim 7, further comprising determining a concentration of the breast milk sample by a technique selected from the group consisting of visual inspection, application of a light source, application of an electrochemical source, application of a sound source, application of a flow counter, application of a speed measurement device, application of a drop counter, application of a drop timer, and combinations thereof.

10. The method of claims 7-9, further comprising at least one of
adding at least one dye to the breast milk sample to aid in visualization, wherein the dye is selected from the group consisting of: litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino)azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarine Yellow R, FD&C Red 3, FD&C Red 40, FD&C Yellow 5, FD&C Yellow 6, FD&C Blue 1, FD&C Blue 2, FD&C Green 3, Caramel Coloring, Annatto, Chlorella, Cochineal, Beet Juice, Saffron, Paprika, Tumeric, Anthrocyanin, Chlorophyll, beta-Carotene, B-Apo-8′-Carotenal, Canthaxanthin, Carrot Oil, Cottonseed Flour, Ferrous Gluconate, Grape Extract, Riboflavin, Carminic Acid, Titanium Dioxide, salts thereof, and combinations thereof; and
adding at least one redox active species to increase the conductivity of the milk sample to aid in detection and subsequent determination of the content wherein said species is selected from the group consisting of NaCl, KCl, NaBr, NaI, KBr, KI, ferrocene; tris(2,2′-bipyridine)ruthenium (II); and tris(2,2′-bipyridine)osmium (II), derivatizied ferrocene, methyl violagen, polythiophene, polyanaline, polypyrrole, ruthenium trisbypridine, transitional metal complex, conducting polymer, and combinations thereof.

11. The method of claim 7, further comprising
adding the sample of breast milk to a vessel constructed and arranged to hold the sample of breast milk to be assayed; and
inserting the vessel into a device comprising a detection circuitry.

12. A device for testing the caloric or fat content of a breast milk sample comprising:
a loading reservoir for holding the sample;
a detection cell constructed and arranged to allow passage of the sample thereby producing a milk and surface interaction;
a catch reservoir for retaining an efflux of the sample; and
a detection circuitry constructed and arranged to measure a physical property of the sample and display a response.

13. A device for testing a body fluid for analytes comprising:
a vessel constructed and arranged to hold a sample of the body fluid;
a cap for closing the vessel; and
at least one material selected from the group consisting of:
a detecting pH sensitive dye,
a colorant dye,
a base,
a solvent to improve solubility,
a detecting enzyme,
a substrate for an enzyme, and
a metabolic activity detecting agent,
wherein the at least one material is contained in at least one of the vessel, the cap, a crushable ampoule, and combinations thereof.

14. The device of claim 13, further comprising a component selected from the group consisting of a medicament, colorant, flavoring, scent, fibrous additive, antioxidant, thickener, plasticizer, preservative, stabilizer, and combinations thereof in the at least one of the vessel, the cap, a crushable ampoule, and combinations thereof.

15. The device of claim 13 that is constructed and arranged to analyze a property of the body fluid selected from the group consisting of:
an acidity of the sample to determine if it is spoiled comprising a pH sensitive detecting agent dye and a base where an incomplete acid-base reaction occurs between the base and the acid in the fluid such that the detecting agent changes;
an endotoxin load in the sample to count bacterial levels whereby an enzyme/compound is used to react to the endotoxins in live/dead bacteria;
a metabolic activity of the sample to detect spoilage whereby a metabolic detecting agent is used to determine the amount of active bacteria present in the sample; and
a concentration of a metal in the sample by using a detecting enzyme/substrate combination that is effected by the presence of a metal wherein said metal is selected from the group consisting of mercury, inorganic mercury, organic mercury, mercury chloride, mercury bromide, mercury acetate, mercury iodide, lead, lead chloride, lead acetate, lead bromide, lead iodide, antimony (Sb), arsenic (As), cadmium (Cd), calcium(Ca), chlorine (Cl), chromium (Cr), cobalt (Co), copper (Cu), fluorine (F), iodine (I), iron (Fe), lead (Pb), magnesium (Mg), manganese (Mn), mercury (Hg), molybdenum (Mo), nickel (Ni), phosphorus (P), potassium (K), selenium (Se), sodium (Na), tin (Sn), vanadium (V), and zinc (Zn).

16. The device of claim 15, wherein the detecting agent is selected from the group consisting of
a tetrazolium salt, resazurin, methyl blue, dodecylresazurin, RedoxSensor Red, Limulus amoebocyte lysate, litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino)azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarin Yellow R, salts thereof, ferrocene; tris(2,2′-bipyridine)ruthenium (II); and tris(2,2′-bipyridine) osmium (II), derivatizied ferrocene, methyl violagen, polythiophene, polyanaline, polypyrrole, ruthenium trisbypridine, transitional metal complex, conducting polymer, and combinations thereof,
and wherein the detecting enzyme selected from the group consisting of mercuric reductase, l-lactate dehydrogenase, invertase, δ-aminolevulinate dehydrogenase, pyruvate dehydrogenase, alkaline phosphatase, horseradish peroxidase, caspase, and urease, or an oxidoreductase, transferase, hydrolase, lyase, isomerase, ligase, and combinations thereof,
and wherein the substrate is selected from the group consisting of urea, NADPH, lactate, pyruvate, sucrose, δ-aminolevulinate acid, para-nitrophenyl phosphate, 2-2′-azino-di-(3-ethylbenz-thiazoline sulfonic acid), o-phenylenediamine, tetramethylbenzidine, a dye bound to the tetrapeptide sequence aspartic acid-glutamic acid-valine-aspartic acid, and combinations thereof,
and wherein the solvent is selected from the group consisting of pentane, cyclopentane, hexane, cyclohexane, benzene, toluene, 1,4-Dioxane, chloroform, diethyl ether, dichloromethane, tetrahydrofuran, ethyl acetate, dimethylformamide, acetonitrile, dimethyl sulfoxide, formic acid, n-butanol, isopropanol, n-propanole, ethanol, methanol, xylene, ethylene glycol, water, and combinations thereof,
and wherein the base is selected from the group consisting of NaOH, KOH, LiOH, Ca(OH)₂, Ba(OH)₂, Mg(OH)₂, ammonium hydroxide, ammonium citrate, hydroxylamine, pyridine, imidazole, trisamine, triethylamine, NH3, diisopropylethylamine, alanine, dimethylamine, ethylamine, hydrazine, methylethanolamine, methylamine, azetidine, pyrrolidine, piperidine, dimethylethanolamine, diethylamine, aniline, and trimethylamine, and combinations thereof.

17. The device of claim 15, further comprising at least one of
a dye added to the milk sample to aid in visualization selected from the group consisting of litmus, bromophenol blue, bromophenol red, cresol red, α-naphtholphthalein, methyl purple, thymol blue, methyl yellow, methyl orange, methyl red, bromcresol purple, bromocresol green, chlorophenol red, bromothymol blue, phenol red, cresol purple, Creosol red, thymol blue, phenolphthalein, thymolphthalein, indigo carmine, alizarin yellow R, alizarin red S, pentamethoxy red, tropeolin O, tropeolin OO, tropeolin OOO, 2,4-dinitrophenol, tetrabromphenol blue, Neutral red, Chlorophenol red, 4-Nitrophenol, p-Xylenol blue, Indigo carmine, p-Xylenol blue, Eosin, bluish, Epsilon blue, Bromothymol blue, Thymolphthalein, Titan yellow, Alkali blue, 3-Nitrophenol, Bromoxylenol blue, Crystal violet, Cresol red, Congo red, Bromophenol blue, Quinaldine red, 2,4-Dinitro phenol, 2,5-Dinitrophenol, 4-(Dimethylamino)azobenzol, Bromochlorophenol blue, Malachite green oxalate, Brilliant green, alizarin sodium sulfonate, Eosin yellow, Erythrosine B, α-naphthyl red, p-ethoxychrysoidine, p-nitrophenol, azolitmin, neutral red, rosolic acid, α-naphtholbenzein, Nile blue, salicyl yellow, diazo violet, nitramine, Poirrier's blue, trinitrobenzoic acid, Congo red, Azolitmin, Neutral red, Cresol Red, Alizarin Yellow R, FD&C Red 3, FD&C Red 40, FD&C Yellow 5, FD&C Yellow 6, FD&C Blue 1, FD&C Blue 2, FD&C Green 3, Caramel Coloring, Annatto, Chlorella, Cochineal, Beet Juice, Saffron, Paprika, Tumeric, Anthrocyanin, Chlorophyll, beta-Carotene, B-Apo-8′-Carotenal, Canthaxanthin, Carrot Oil, Cottonseed Flour, Ferrous Gluconate, Grape Extract, Riboflavin, Carminic Acid, Titanium Dioxide, salts thereof, and combinations thereof; and
a redox active species to increase the conductivity of the milk sample to aid in detection and subsequent determination of the content selected from the group consisting of NaCl, KCl, NaBr, NaI, KBr, KI, ferrocene; tris(2,2′-bipyridine)ruthenium (II); and tris(2,2′-bipyridine) osmium (II), derivatizied ferrocene, methyl violagen, polythiophene, polyanaline, polypyrrole, ruthenium trisbypridine, transitional metal complex, conducting polymer, and combinations thereof.

18. A method of testing a body fluid for analytes comprising:
providing a vessel constructed and arranged to hold a sample of the body fluid;
providing a cap for closing the vessel; and
providing at least one material selected from the group consisting of:
a pH-sensitive dye,
a colorant dye,
a base,
a solvent,
an enzyme,
a substrate, and
a metabolic activity indicator, wherein the at least one material is contained in at least one of the vessel, the cap, a crushable ampoule, and combinations thereof;
adding the body fluid to the vessel;
mixing the body fluid and the at least one material to provide a response; and
analyzing the response.

19. The adapter of claims 3-5, device of claim 12, or device of claim 13, further comprising sterilizing the adapter or device using a technique selected from the group consisting of visible light irradiation, ultraviolet light, electron-beam radiation, gamma-radiation, chemical techniques, physical techniques including wet and dry heating, and combinations thereof.

20. A kit containing one or more of the adapter of claims 3-5, device of claim 12, and device of claim 13, further comprising at least one of:
a delivery system selected from to the group consisting of a syringe, spoon, cup, trough, pipette, dropper, and capillary tube;
a logbook for recording results;
a chart for plotting results;
instructions and a URL for a website where results can be interfaced;
a desiccant;
an antioxidant,
means for achieving an inert atmosphere;
packaging;
means for light blocking; and
instructions.
